(19) United States
(12) Patent Application Publication
(10) Pub. No.: US 2022/0344060 A1
(43) Pub. Date: Oct. 27, 2022

(54) SYSTEMS AND METHODS FOR ASSESSING OUTCOMES OF THE COMBINATION OF PREDICTIVE OR DESCRIPTIVE DATA MODELS

(71) Applicant: THE BRIGHAM AND WOMEN'S HOSPITAL, INC., Boston, MA (US)

(72) Inventors: Bruce S. KRISTAL, Cohasset, MA (US); Matthew J. SNIATYNSKI, Somerville, MA (US); Derbiau Frank HSU, Weston, MA (US)

(21) Appl. No.: 17/640,729

(22) PCT Filed: Sep. 4, 2020
(86) PCT No.: PCT/US20/49577

§ 371 Date: Mar. 4, 2022

Related U.S. Application Data
(60) Provisional application No. 62/895,846, filed on Sep. 4, 2019.

(51) Int. Cl. G16H 50/50; G16H 40/63

(52) U.S. Cl. CPC G16H 50/50; G16H 40/63

(57) ABSTRACT

An improved patient monitoring system can include a processor device, a display, a first sensor in communication with the processor device, the first sensor being at least one of an electrocardiogram sensor, a pressure sensor, a blood oxygenation sensor, an image sensor, an impedance sensor, or a physiological sensor. The system can include a second sensor in communication with the processor device, the second sensor being a physiological sensor. The processor device can be configured to utilize the first accuracy, the second accuracy, the first correlation, the second correlation to determine a recommendation for fusing the first data model with the second data model.

### CROSS-REFERENCE TO RELATED APPLICATIONS

This application claims priority to U.S. Patent Application No. 62/895,846 filed Sep. 4, 2019, and entitled, “Ranks underlie outcome of combining classifiers: quantitative roles for Diversity and Accuracy,” which is hereby incorporated by reference in its entirety.

### STATEMENT REGARDING FEDERALLY SPONSORED RESEARCH

This invention was made with government support under R01-HL-132556 and R01-HL-140335, both awarded by the National Institutes of Health. This invention was also made with government support under the federal grant number 1P01CA168530. The government has certain rights in the invention.

### BACKGROUND

The use of large datasets and other data models is central to many aspects of modern business, science, and medicine. In many cases, it may be advantageous to combine multiple models for power or robustness, but it is well-recognized that realizing these potential gains cannot be guaranteed—especially when the input models cannot be appropriately weighted or the resulting fusion models cannot be properly cross-trained or cross-validated. Similarly, in other cases, combining multiple models may not provide any advantages, and rather may create downsides for the entire system (e.g., unneeded calculations, degraded accuracy, costs associated with collecting non-informative data, etc.). Thus, it would be desirable to provide systems and methods for assessing outcomes of the combination of predictive or descriptive mathematical systems that can improve efficiencies and accuracy of various systems.

### SUMMARY OF THE DISCLOSURE

Combining classifier systems can potentially improve performance, but performance outcomes have historically been proven very difficult to predict. Performance most commonly improves when the classifiers have high individual performance (“Accuracy”) and are “sufficiently different” (“Diversity”), but the individual and joint quantitative influence of these factors on the final outcome still remains unknown. Some non-limiting examples of the disclosure addresses these (and other) issues. Simulated data was utilized to develop the DIRAC Framework (DIversity of Ranks and ACcuracy), which as described below, can accurately predict outcome of both score-based fusions originating from exponentially-modified Gaussian distributions, and rank-based fusions, which are inherently distribution independent. This framework was validated using biological DXA and MRI-based imaging data. The DIRAC framework is domain independent and has expected utility in far-ranging areas such as clinical biomarker development/personalized medicine, clinical trial enrollment, insurance pricing, portfolio management, and sensor optimization.

Some aspects of the disclosure provide systems and methods for Optimizing the Accuracy and Computational Efficiency of Information Fusion across Multiple Domains.

The foregoing and other aspects and advantages of the disclosure will appear from the following description. In the description, reference is made to the accompanying drawings which form a part hereof, and in which there is shown by way of illustration a preferred configuration of the disclosure. Such configuration does not necessarily represent the full scope of the disclosure, however, and reference is made therefore to the claims and herein for interpreting the scope of the disclosure.

### BRIEF DESCRIPTIONS OF THE DRAWINGS

FIG. 1 shows a flowchart of different data models and the undetermined relationship between them.

FIG. 2 shows a graphic listing multiple inputs for fusing information.

FIG. 3 shows a flowchart of a single data model that utilizes raw data to extract feature(s), which are used to generate scores and/or rank(s), which are used to form a decision.

FIG. 4 shows a flowchart of a specific example of the configuration of FIG. 3.

FIG. 5 shows a flowchart of an example of fusing (or concatenating) together a first data model (which is first raw data) with a second data model (which is second raw data).

FIG. 6 shows a flowchart of a specific example of the configuration of FIG. 5.

FIG. 7 shows a flowchart of an example of fusing together a first data model (which is a first feature classifier) and a second data model (which is a second feature classifier).

FIG. 8 shows a flowchart of an example of fusing together a first data model (which is a first score or rank classifier) and a second data model (which is a second score or rank classifier).

FIG. 9 shows a flowchart of a specific example of the configuration of FIG. 10.

FIG. 10 shows a flowchart of an example of fusing together a first data model (which is a first decision classifier) and a second data model (which is a second decision classifier).

FIG. 11 shows a flowchart of a specific example of the configuration of FIG. 9.

FIG. 12 shows a graphic illustrating the principle of the different information that can be obtained from one data set from two different data models A and B.

FIG. 13 shows a schematic illustration of a computing system

FIG. 14 shows a flowchart of a process for generating evaluation criteria that can be used to determine a recommendation for or against fusing a pair of data models.

FIG. 15 shows a flowchart of a process for evaluating fusion of pairs of data models.

FIG. 16 shows a graphic illustrating a process used to generate simulated data that includes a plurality of pairs of fused data models.

FIGS. 17A-17C4 shows various graphs illustrating how the simulated data was generated.

FIG. 18 shows graphs of plotted pairs of data models indicating which regions are associated with improved and decreased (or no change) performance after fusion. These use score-based fusions and are related to classification.

FIGS. 19A-19C shows a series of graphics, for particular correlation intervals, illustrating whether or not pairs of fused data models increased the accuracy of the underlying data models within each pair. These use score-based fusions and are related to classification.

FIG. 20 shows a series of graphics illustrating the impact of the number of samples (N) used to generate data models for pairs of fused data models, where the pairs of fused data models can predict whether or not to fuse a pair of data models.

FIG. 21 show graphs of plotted pairs of data models indicating which regions are associated with improved and decreased (or no) performance after fusion. These use rank-based fusions and are related to classification.

FIGS. 22A-22C shows a series of graphics, for particular correlation intervals, illustrating whether or not pairs of fused data models increased the accuracy of the underlying data models within each pair, using, a sample size N of 600 to create each data model within each pair of data models. These use rank-based fusions and are related to classification.

FIGS. 23A-23B shows a series of graphs, for particular correlation intervals, with locally weighted scatterplot smoothing (“LOWESS”) curves generated for each correlation interval.

FIGS. 24A-24B shows a series of graphs, for particular correlation intervals, of a real world data set having a plurality of pairs of data models, each plotted on a particular correlation interval. The LOWESS curves, for each correlation interval as illustrated in FIGS. 23A and 23B, are superimposed on each correlation interval that has the real world data set.

FIG. 25 shows a graph of the combined correlation and change in accuracy for each pair of data models of the real world data set, which shows that more negative correlations are associated with a broader range of differences in AUC that can be used and still see gains in accuracy with fusions.

FIG. 26 shows graphs, for a group of specific correlation slices (or filters), of a continuous scoring system that indicates locations of improved (e.g., shown in blue) or not improved (e.g., shown in red) data model fusions. The data series shown here continues in FIGS. 27 and 28 with additional correlation slices

FIG. 27 shows graphs, for another group of specific correlation slices (or filters), of a continuous scoring system that indicates locations of improved (e.g., shown in blue) or not improved (e.g., shown in red) data model fusions.

FIG. 28 shows graphs, for another group of specific correlation slices (or filters), of a continuous scoring system that indicates locations of improved (e.g., shown in blue) or not improved (e.g., shown in red) data model fusions.

FIG. 29 shows graphs, for a group of specific correlation slices (or filters), of a discrete scoring system that indicates locations of improved (e.g., shown in blue) or not improved (e.g., shown in red) data model fusions. The data series shown here continues in FIGS. 30 and 31 with additional correlation slices

FIG. 30 shows graphs, for another group of specific correlation slices (or filters), of a discrete scoring system that indicates locations of improved (e.g., shown in blue) or not improved (e.g., shown in red) data model fusions.

FIG. 31 shows graphs, for another group of specific correlation slices (or filters), of a discrete scoring system that indicates locations of improved (e.g., shown in blue) or not improved (e.g., shown in red) data model fusions.

FIG. 32 shows an upper graph of the AUC vs. the AUC-Angle, and a lower graph of the within class correlation vs. the rotation angle (e.g., denoted as the “angleBetween”).

FIG. 33 shows a series of graphs of the density vs. value of two data models for the continuous case. Each graph is derived from different sample sizes (e.g., denoted as 100, 200, 600, and 1200), and all the graphs within FIG. 33 have the same overlap fraction of 0.05. The analysis shown here continues in FIGS. 34-39.

FIG. 34 shows a series of graphs of the density vs. value of two data models for the continuous case. Each graph is derived from different sample sizes (e.g., denoted as 100, 200, 600, and 1200), and all the graphs within FIG. 34 have the same overlap fraction of 0.1.

FIG. 35 shows a series of graphs of the density vs. value of two data models for the continuous case. Each graph is derived from different sample sizes (e.g., denoted as 100, 200, 600, and 1200), and all the graphs within FIG. 35 have the same overlap fraction of 0.2.

FIG. 36 shows a series of graphs of the density vs. value of two data models for the continuous case. Each graph is derived from different sample sizes (e.g., denoted as 100, 200, 600, and 1200), and all the graphs within FIG. 36 have the same overlap fraction of 0.3.

FIG. 37 shows a series of graphs of the density vs. value of two data models for the continuous case. Each graph is derived from different sample sizes (e.g., denoted as 100, 200, 600, and 1200), and all the graphs within FIG. 37 have the same overlap fraction of 0.4.

FIG. 38 shows a series of graphs of the density vs. value of two data models for the continuous case. Each graph is derived from different sample sizes (e.g., denoted as 100, 200, 600, and 1200), and all the graphs within FIG. 38 have the same overlap fraction of 0.5.

FIG. 39 shows a series of graphs of the density vs. value of two data models for the continuous case. Each graph is derived from different sample sizes (e.g., denoted as 100, 200, 600, and 1200), and all the graphs within FIG. 39 have the same overlap fraction of 0.75.

FIG. 40 shows a series of graphs. Correlation slices are the correlation between the outputs of the two data models, Con 1 is the Spearman rank correlation of the output of the first data model with the target, and Con 2 is the Spearman rank correlation of the output of the second data model with the target. Blue points show a recommendation for fusion, while red points show a recommendation against fusion. The purple locations indicate a region for net improvement (in accuracy for fusing the two models). Each row of graphs is for a different correlation slice from 0 to 1. Left column shows correlation slice, center column shows qualitative effect of fusion, right column shows quantitative effect of fusion_The analysis continues in FIGS. 41 and 42.

FIG. 41 shows a series of graphs. Correlation slices are the correlation between the outputs of the two data models, Con 1 is the Spearman rank correlation of the output of the first data model with the target, and Con 2 is the Spearman rank correlation of the output of the second data model with the target. Blue points show a recommendation for fusion, while red points show a recommendation against fusion. The purple locations indicate a region for net improvement (in accuracy for fusing the two models). Each row of graphs is for a different correlation slice from 0 to 1. Left column shows correlation slice, center column shows qualitative effect of fusion, right column shows quantitative effect of fusion.

FIG. 42 shows a series of graphs. Correlation slices are the correlation between the outputs of the two data models, Con 1 is the Spearman rank correlation of the output of the first data model with the target, and Con 2 is the Spearman rank correlation of the output of the second data model with the target. Blue points show a recommendation for fusion, while red points show a recommendation against fusion. The purple locations indicate a region for net improvement (in accuracy for fusing the two models). Each row of graphs is for a different correlation slice from 0 to 1. Left column shows correlation slice, center column shows qualitative effect of fusion, right column shows quantitative effect of fusion.

### DETAILED DESCRIPTION

Limitations in our ability to optimally combine prediction and/or classification algorithms directly—due in large part to our incomplete and non-quantitative understanding of what drives the success or failure of this type of information fusion—is a fundamental roadblock to fully unleashing the power inherent in big data and current informatics. Data-driven analyses and sophisticated modelling approaches have revolutionized modern business, science, and medicine. The modelling methods applied in these domains span the breadth of statistical and mathematical knowledge, from straight-forward parametric methods such as linear or logistic regression, through more complex non-parametric methods such as random forest classification or support vector machines, to ensemble classifiers and the very latest deep-learning neural networks. If data are plentiful and models are directly comparable, then the above approaches continue to be appropriate and powerful methods of choice. When these conditions cannot be met, however, as is often the case in real world problems such as medical and financial risk prediction, such approaches are known to have limitations. In particular, such limitations often make it impossible to build single models that optimally capture the richness of a given dataset.

Combining multiple, weaker models has the potential to improve outcomes. Similar to personal opinions, different data streams and data models may partially or totally disagree. The potential utility—and complexity—of taking multiple opinions—mathematical or personal—into account has long been recognized and has been formally considered since at least the 1700's. Today, this process is known by many names, including Information Fusion (“IF”), and can occur at three conceptually different levels, pre-training (typically called data- or feature-level fusion), post-training (known by many names, e.g., system or model level fusion), and post-decision (e.g., voting, or decision fusion), each of which has specific, distinct, and substantial advantages and disadvantages. Data/feature level fusion (e.g., merging datasets) is the simplest in execution (e.g., concatenating two or more sets of data, such as measurements including genes and clinical parameters), is well-understood, and such approaches lend themselves to the subsequent use of well-defined and powerful mathematical techniques (e.g., ensemble classifiers, penalized regression, classification trees, or other familiar statistical/informatics analyses and approaches)—a combination that generally makes them the approach of choice for IF. Unfortunately, these techniques are often unusable due to data limitations or distributional assumptions. For example, generally this approach demands appropriate scaling, requires appropriate weights to be found (e.g., consider weighting 5,000,000 SNPs (genetic markers) vs 10 clinical parameters), and also must address other mismatch problems such as categorical vs continuous variables. As another example, data fusion approaches also inherently expand the multiple comparison and n vs p problems (number of observations vs number of variables), increasing the chance of over-fitting and the requirement for larger (often prohibitively large) datasets for training, optimization, and testing.

Decision fusion approaches, such as voting, operate at the other end of the spectrum, and involve the integration of the final output of a set of data models. For example, if a pool of different classifier systems (potentially trained on different datasets) are to predict whether a set of patients have a disease or not, decision fusion will involve each classifier voting on each patient's status. Decision fusion-based approaches inherently solve many of the problems seen in data fusion (e.g. mismatched data types). Decision fusion has been extensively studied, particularly in the context of voting, and remains an active area of research. Aside from these advantages, however, Decision fusion can have problems including the inability to carry forward appropriate weights or adequately reflect the level of certainty of the underlying models (e.g., Decision fusion cannot naturally account for the certainty/confidence of individual classifiers). Decision fusion also has no mechanism for accounting for potential classifier complementarity (e.g., multiple similar models can overwhelm minority viewpoints). System fusion, which is also known as model level fusion, post-training fusion, multiple system classifier fusion, and the like, on the other hand, combines data models at a level between these two, after the models have been trained on the input data, but before a classification decision has been made. This involves combining appropriately-scaled intermediate output of the classification systems in question before thresholding has taken place (e.g., before the intermediate output is transformed into a decision). Fusion carried out at this level has the potential to be more flexible and powerful compared to the other two, as differences in dataset composition/distribution are taken care of by the systems themselves (before fusion), and the scaled score given to each sample by each system retains some of the “certainty” of that system, as pertains to that sample. Of the three fusion approaches, however, system fusion is the least understood, but could address the above short-comings of the other systems

It has been recognized that system fusion (e.g., MSC-fusion) performance is, in part, influenced by the performance of the individual models and the diversity between them. For example, for the fused system to be an improvement over its constituent systems, it is believed that these constituents must be both “good” enough and “different” enough, but the current literature seems to favor and reflect a general emphasis on the importance of accuracy. Despite decades of work, the quantitative details that involve this relationship between “good” enough and “different” enough still remain elusive. In fact, some current approaches for system fusion that have been developed were found to work well in some domains but not in others, providing no significant answers to this relationship. Importantly, these approaches (and others) provide no reasonable answers or significant steps to answering the generalized problem of determining whether two systems should be fused. Thus, this still remains a difficult problem.

The limitations in MSC derive from concerns similar to those underlying the No Free Lunch (“NFL”) theorems in optimization and machine learning, which were formalized in the 1990's. The NFL theorems indirectly indicate that, in the general case (across all possible application domains), the benefit, or lack thereof, derived from the fusion of two models is inherently unknowable. For every situation where a particular fusion would be advantageous, there will be a situation in which it is disadvantageous. Given this general constraint, it is important to note that some understanding has been gained in domain-specific subclasses of information fusion, such as the use of linear combinations and rank-score diversity (“SRD”) in information retrieval, and score-rank diversity in in silico drug screening. In both these situations the signal of interest tends to lie in a single tail of the distributions of the model outputs, giving probabilistic structure that can guide fusion approaches and circumvent the NFL limitations. Accuracy improvements in these approaches have been typically in the 70% range, although isolated fully accurate predictions have been observed. While there have been some advancements, the details, for example the individual and joint quantitative influence of these factors on the final outcome, remain unknown. Indeed, even whether fusing their expertise is likely to improve performance remains unknown.

Previous approaches that have aimed to solve this problem have been largely inadequate (e.g., being incomplete, incorrect, or both). As described above, NFL provides an absolute prohibition in predicting fusions under circumstances where the domain is unknown. However, work in the decade after the NFL theorems were codified (either directly or indirectly) began to work around its limitations by either utilizing subject domain knowledge (e.g., information retrieval looks for a small number of outliers on one side of the distribution in a vast sea of noise), or mathematical domain knowledge (e.g., the mathematical structure of score rank plots). For example, one previous approach showed that, in information retrieval, accuracy was improved for score fusions by using similar accuracy models that were diverse by Kendall's Tau. This approach defined accuracy similarity based on the ratio of the accuracies of the two models, and thus could not observe the secondary effect of absolute accuracy, nor was their dataset rich in negative Kendall's Tau data points, which were ultimately ignored. This approach was about 68-74% accurate in predicting whether a fusion would benefit, although this approach did not consider rank fusions. Another later approach demonstrated that models having specific characteristics, specifically, agreement on correct items and disagreement on incorrect items, could be usefully fused. This highlighted the use of diversity as a means to move the negatives away from the positives, recognized group specific diversity (but only within one group), but was non-quantitative, did not recognize the utility of diversity within both groups, and was largely applicable on to information retrieval or other cases where there were a small number of positives at one end of the scale. An even later approach, provided a geometric argument that, in a multidimensional space, MSC-fusion can be modeled as two line segments proportional to the accuracy of the individual classifiers and the angle between them, which is related to diversity—similar arguments are extended in a later approach to this, which handles rank fusions as though they were continuous variables. Such models are extendable to explain the power of negative correlations that we observe, although neither group recognizes/addresses either this or the intra-group nature of the correlation effect, both of which are essential aspects of non-limiting examples of this disclosure, as described below. Yet another approach showed that confidence could be used to assess the weights in fusion. This is related to both the “N” and the diversity aspects of non-limiting examples of this disclosure, although they do not address false confidence, and are largely focused on single point predictions and communication. Thus, the fundamental of our approach is grounded in sound mathematical work, including formal proofs, that have been previously conducted in the field of IF, but non-limiting examples of this disclosure extend these far beyond the previous approaches, and show that this has broad applicability to problems involving the entire range of a distribution, demonstrates utility (e.g., near 100% accuracy vs 68-74%), leverages the involvement of ranks, and broadly puts all pieces together.

Some non-limiting examples of the disclosure, rather than focusing on a domain-specific context (e.g., information retrieval) to understand and improve system-level fusion, take the converse approach, and study one particular type of system fusion across as many different input data distributions as possible, so as to identify characteristics of the input systems and their data that may identify, in advance situations, where fusion is likely to be beneficial. For example, quantile classification systems similar to those that are commonly encountered in biological work were the systems that were focused on, which assign a monotonically increasing score to each sample to be classified and measure class separation performance using the area under the receiver operating characteristic curve (“AUROC”). The diversity between these systems using common correlation metrics (e.g., Pearson correlation) were measured. Pairwise fusions were examined by averaging the scores of the two systems across each sample.

Some non-limiting examples of the disclosure provide systems and methods that assess whether a proposed combination of predictive or descriptive mathematical systems (e.g., a fusion of the models) will improve their accuracy. For example, some non-limiting examples of this disclosure assess the likely outcome (improved accuracy, no change, worsen accuracy) of information fusions prior to doing them. This level of informatics, which can be termed as system level fusion, sits between the levels often referred to as data fusion and decision level fusion (or voting). System level fusion is well-recognized to offer unique opportunities for the analysis and utilization of data sets of multiple types at all scales. As described above, previous approaches at this level can be unpredictable, and at best rarely exceed 70% accuracy under very narrow, pre-defined conditions. In contrast, non-limiting examples of the disclosure offer distribution dependent and independent approaches having very high accuracy (>99.7% in a blinded test series of >100,000 biological measurements for distribution independent).

Other non-limiting examples of the disclosure address other certain types of usages (e.g., applications), which can turn exponentially complex computer calculations—which have been known to be a major limitation in one “level” of informatics and modeling for decades—into sublinear computing requirements. In fact, some non-limiting examples can address a very broad subclass of these problems. For example, the systems and methods of this disclosure rely on an embedded mathematical “engine,” described in more detail below, that has far reaching applications. For example, this can be helpful for use in medical devices and other point of care devices (e.g., where prompt accurate assessments can save lives), in other cases such as stock trading where millisecond gains have large financial implications, and in cases that allow for the ability to explore larger search spaces offers improved performance characteristics (e.g., in arbitrage, insurance, and the like). Broad uses also exist in areas that offer improvements to computing technology, such as cell phones (e.g., for improving speed and reduced power consumption), and systems that have many sensors (e.g., either with or without an active response element).

Some non-limiting examples of the disclosure can result in large scale gains (or improvements) for systems (e.g., a computing system or device), while other can result in small shifts. However, regardless, any shifts (e.g., small or large improvements) can mean that you “win” more often, when you “win”, you “win” more, and when you “lose”, you “lose” less. Some non-limiting examples also allow for a shift in optimal f-based strategies in stocks.

Some non-limiting examples of the disclosure can result in an expanded search space, which can consider more options in similar time (e.g., in financial options, you can not only consider spreads within a group, e.g., financials, but you can consider all available futures contracts (asset, time, strike, and the like) essentially simultaneously). Similarly, you can balance portfolios on an unlimited number of scenarios.

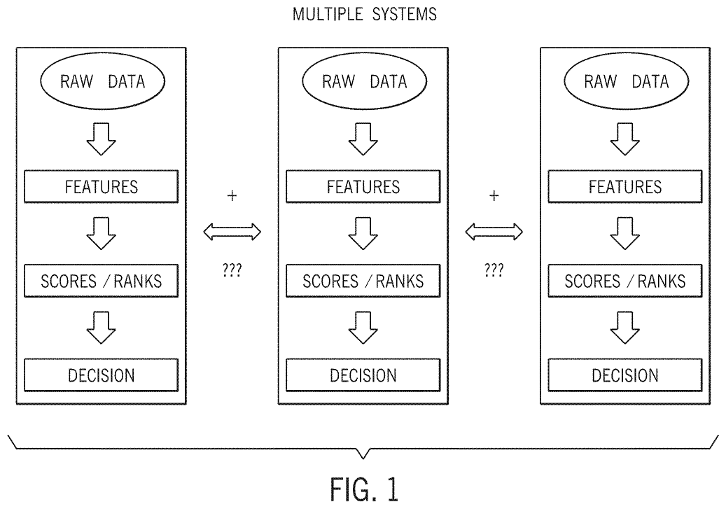

FIG. 1 shows a flowchart of different data models and the undetermined relationship between them. In particular, FIG. 1 shows that, conventionally, fusion of multiple data models area currently difficult (if not impossible) to predict.

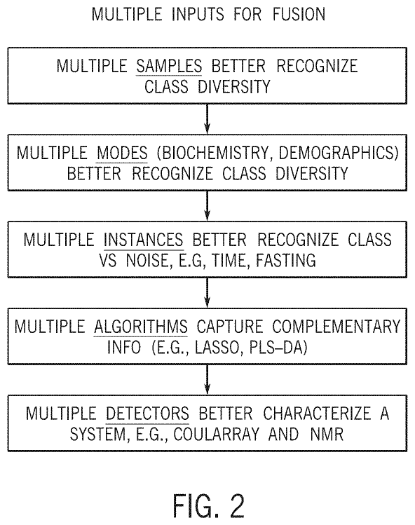

FIG. 2 shows a graphic listing multiple inputs for fusing information. In particular, FIG. 2 shows that at nearly every level (e.g., raw data, feature classifiers, score/rank classifiers, and decision classifiers) have the potential to perform better with multiple (and combined) data models.

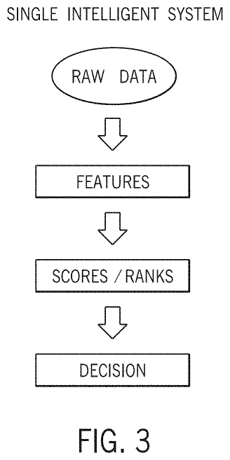

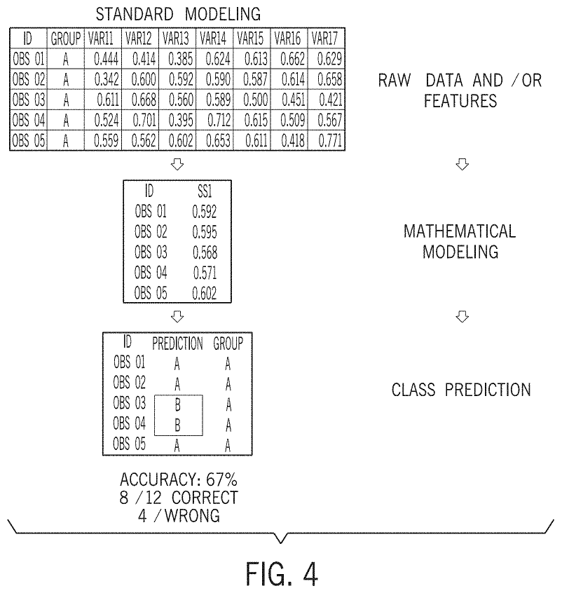

FIG. 3 shows a flowchart of a single data model that utilizes raw data to extract feature(s), which are used to generate rank(s), which are used to form a decision. FIG. 3 illustrates three main layers: one layer represents raw data, and/or features. The second main layer is the output of a scoring system (e.g., the score following a simple statistical model such as regression). It is noted that the scoring system may be as simple as, report the value of variable n, and matches the observation identification and to its cognate score. The third main layer is the class prediction list (e.g., whether, based on the score above the observation is assigned to Class 1 or Class 2 (noted as A,B, for example in FIG. 4)). As above, this matches the observation identification to its cognate predicted class.

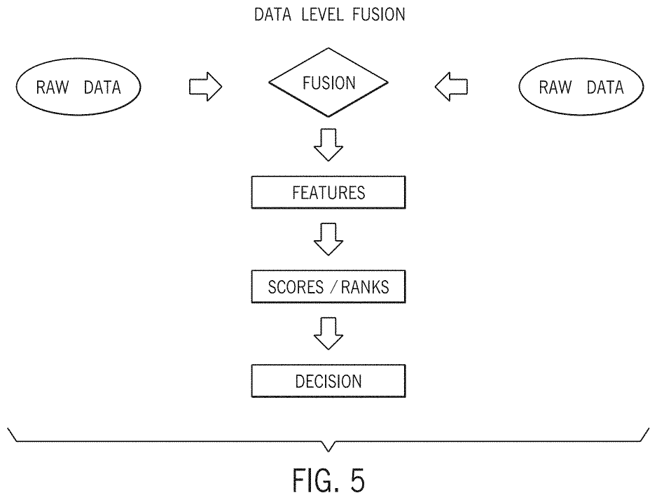

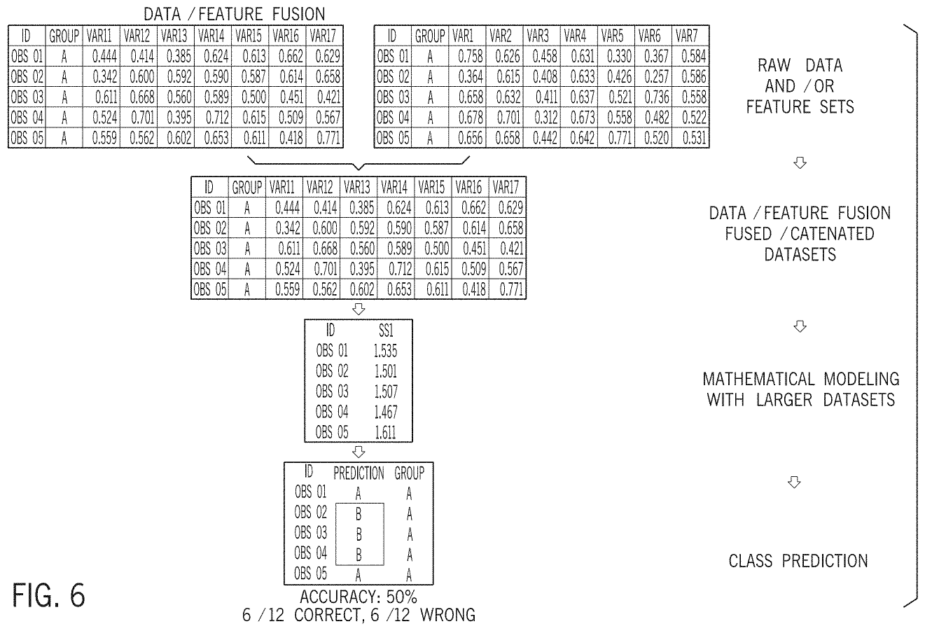

FIG. 5 shows a flowchart of an example of fusing (or concatenating) together a first data model (which is first raw data) with a second data model (which is second raw data). FIG. 6 shows a flowchart of a specific example of the data fusion configuration of FIG. 5.

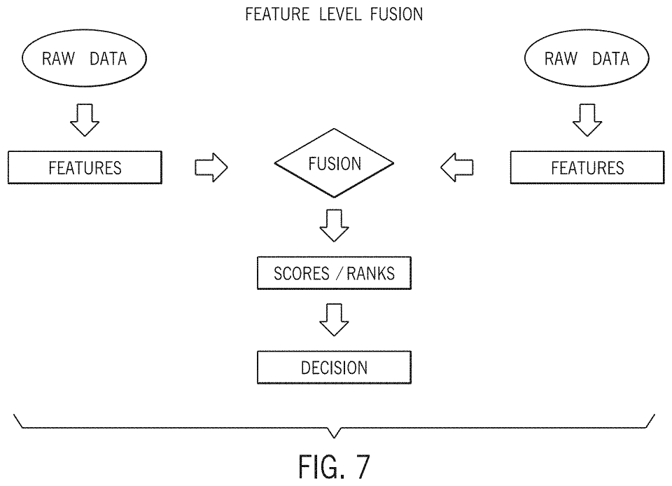

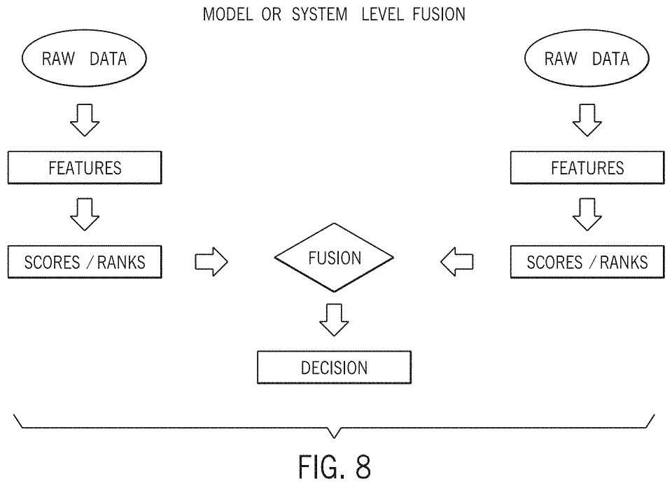

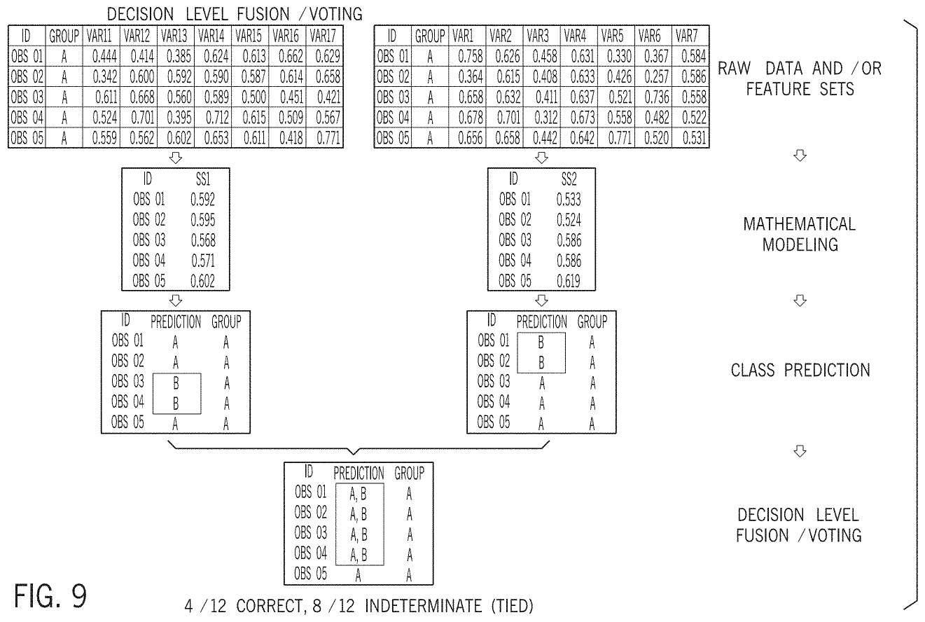

FIG. 7 shows a flowchart of an example of fusing together a first data model (which is a first feature classifier) and a second data model (which is a second feature classifier). FIG. 8 shows a flowchart of an example of fusing together a first data model (which is a first score or rank classifier) and a second data model (which is a second score or rank classifier). FIG. 9 shows a flowchart of a specific example of the configuration of FIG. 7 or FIG. 8.

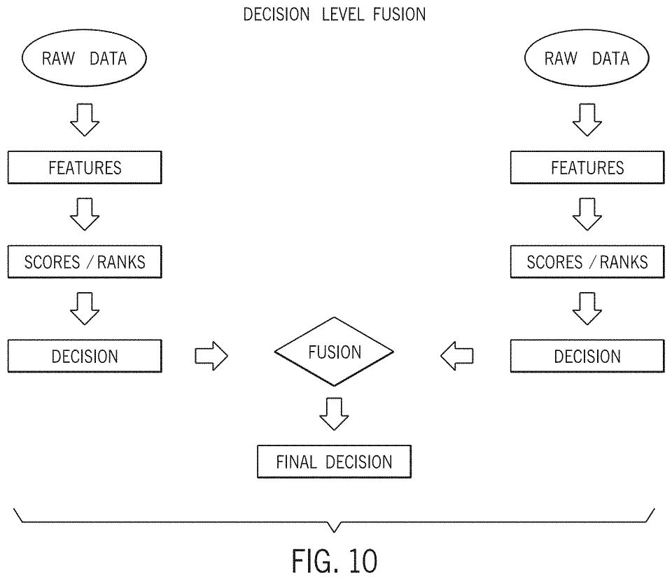

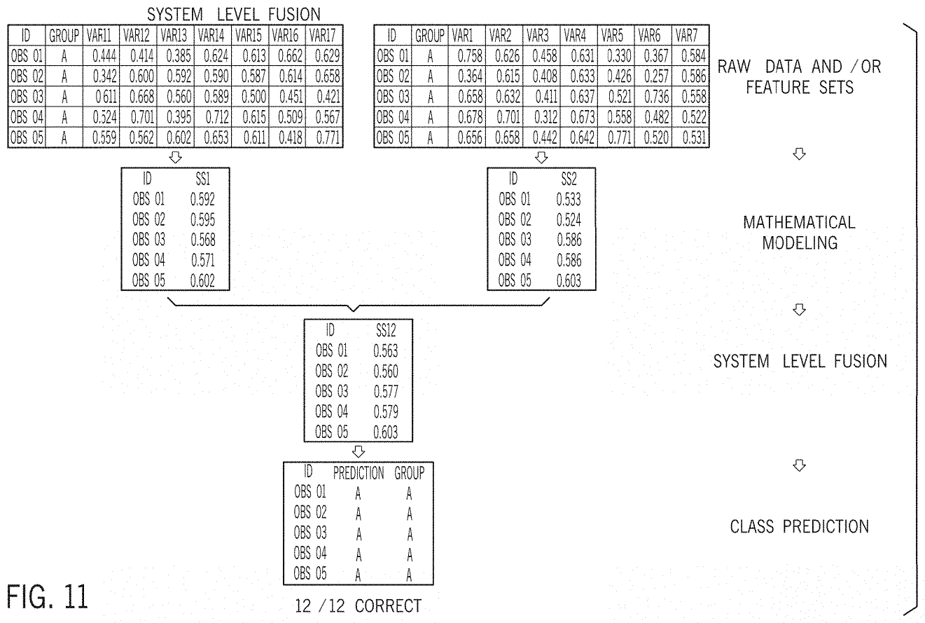

FIG. 10 shows a flowchart of an example of fusing together a first data model (which is a first decision classifier) and a second data model (which is a second decision classifier). FIG. 11 shows a flowchart of a specific example of the configuration of FIG. 10.

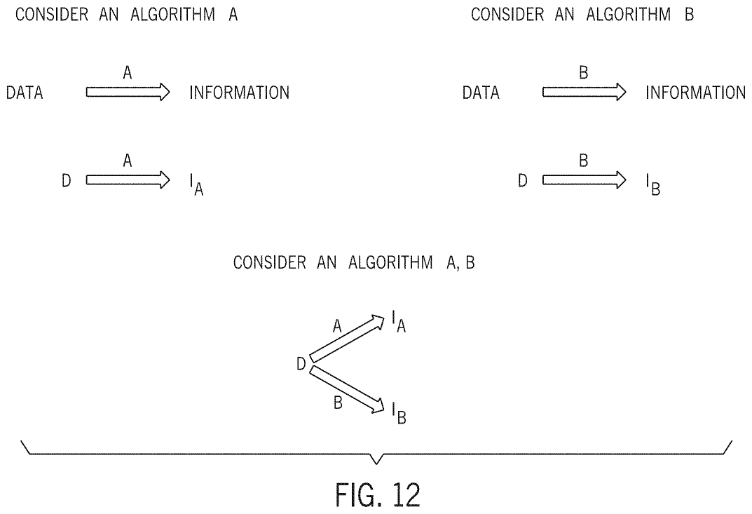

FIG. 12 shows a graphic illustrating the principle of combining data models A and B. Given the systems A, B which transform Data D into information, I (I_{A}, I_{B}), it is questions whether or not instances when system “A” is “better” (e.g., I_{A}>I_{B}) than another specific system “B” all, or at least most of the time. According to non-limiting examples herein, the answer to this question is still no (e.g., by the previously formulated no free lunch theorems that broadly states that for any number of times algorithm A is better than algorithm B, there is an equal number of times when algorithm B is better than A, and vice versa). However, consider two systems A and B, and a third model (or algorithm) C, where C=A+B. It is questioned whether C>A, C>B, or both, or neither. But, according to the no free lunch theorems, there must still be indeterminate, otherwise we could replace B in the above example with A+B such that I_{A+B}>I_{A}.

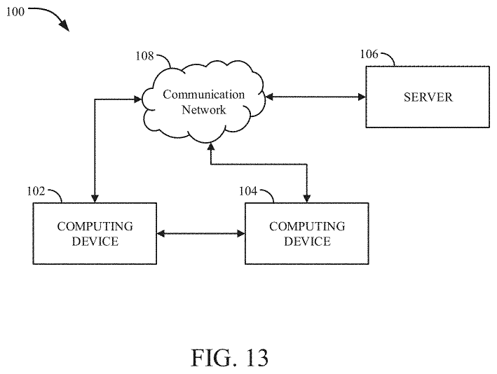

FIG. 13 shows a schematic illustration of a computing system 100 for evaluating fusion of a pair of data models. The system 100 includes a computing device 102 that, as will be described, can present visual information to a user (e.g., a report of the generation indicating the recommendation for (or against) fusion of the pair of models), can receive user inputs, which can adjust operation of another system, such as the computing device 104 based on the recommendation. As shown, the computing system 100 can include a computing device 104, and a server 106. All components within the computing system 100 (and others, such as other systems described above), can all communicate with each other via the communication network 108. In some cases, and as shown, the computing device 102 can directly communicate with the second, optional computing device 104. The communication abilities of the computing devices 102, 104, and the server 106 can include the transmission (and receiving) of data, instructions, and the like, between each other. Additionally, the computing device 102 can store data in and receive data from (e.g., calculated curves for storage in a database, look-up table, and the like) the server 106.

In some non-limiting examples, the computing devices 102, 104, and the server 106 can take any of a variety of forms, including traditional “computer” systems (desktop, laptop), mobile device (tablet or phone). In this way, the computing devices 102, 104 can include a processor device, memory, communication systems, a display, inputs (e.g., a mouse, a keyboard, touch screen or the like, to provide a user input, other sensors, such as physiological sensors, anatomical sensors, etc., communication systems, power sources, while the server 106 can include processor devices, memory, power sources (e.g., power supplies), communication systems, other inputs, and the like.

As described in more detail below, a suitable computing device (e.g., the computing devices 102, 104) can evaluate a pair (or more pairs) of data models to determine a recommendation for fusion of the pair of models. This recommendation can then be used to adjust an operation of a system that is in communication with the suitable computing device. For example, in some non-limiting examples, the suitable computing device can fuse (or reject the fusion of) the pair of data models, for improved efficiency of the suitable computing device (e.g., improved computational efficiency, power allocation, and the like), or the suitable computing device can cause another computing device to change the operation of the system defined by the computing device. For example, with the another computing device having a plurality of sensors each of which can be utilized to determine a particular characteristic (e.g., if a human is present), can be caused (by the suitable computing device) to prevent either or both of data acquisition from a particular sensor (e.g., within the plurality of sensors), or data calculations (or manipulations) for data acquired from the particular sensor (based on the recommendation). In some specific cases, the computing device can be (or form part of) a patient monitoring system, a patient evaluation system, an autonomous vehicle, a semi-autonomous vehicle, or other examples detailed throughout this disclosure. In some cases, some non-limiting examples can be directed to methods for improving the computational efficiency of a computing device (or a computing system), which can generally improve computer technology.

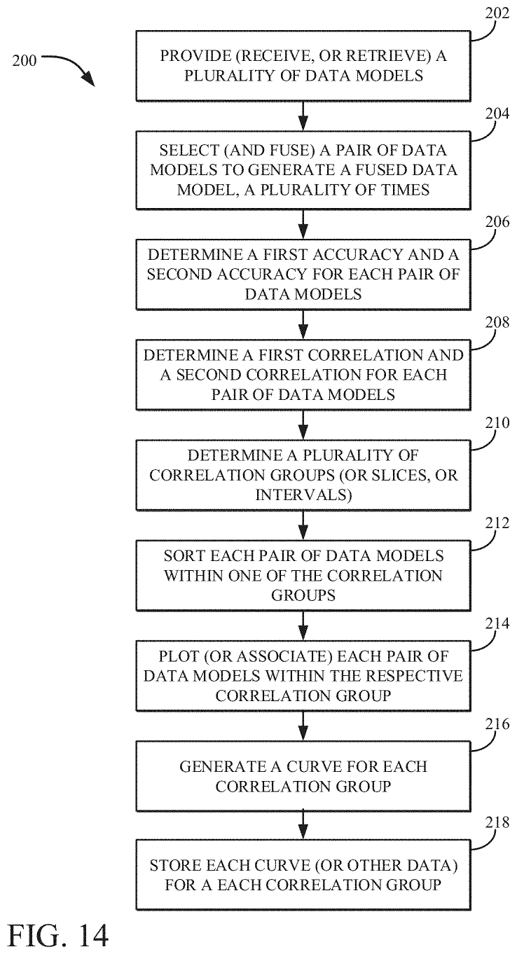

FIG. 14 shows a flowchart of a process 200 for generating evaluation criteria that can be used to determine a recommendation for (or against) fusing a pair of data models. In some configurations, process 200 can be implemented entirely (or partially) on a suitable computing device (or devices), such as the computing devices 102, 104, server 106, or generally a computing system (e.g., the computing system 100).

At 202, process 200 can include providing (receiving, or retrieving, such as from memory) a plurality of data models to the computing system, or computing device. In some configurations, the data model can be any numerical value or output, such as an output of an ensemble classifier, or a single blood glucose measurement. In some configurations, the data model can each be a feature classifier, which is constructed using sample data. In some configurations, the data model can each be a score or rank classifier, which is constructed using sample data to extract feature(s), which are used to determine the score or rank classifier. In some configurations, the data model can be a class predictor (or a decision classifier, or a voting classifier), which is constructed using sample data to extract feature(s), which are used to construct the score or rank classifier(s), which are then used to construct the class predictor. In some non-limiting examples, all the data models are representative of both a first class and a second class. In other words, each data model is capable of classifying observations (e.g., a subject, and the like) into the first class and the second class. Thus, a score classifier can determine scores for each observation, and a rank classifier can determine ranks for each observation.

In some non-limiting examples, a data model within the pair of data models (or both) can be ranks, sets of ranks, rank-based models, and the like. In some non-limiting examples, the use of ranks can be desired for particular applications, such as prioritizing enrollment, recruitment, inclusion exclusion, and the like, which can be, for example, determining specific individuals into a clinical trial, stocks into a portfolio, and the like. In some cases, rank combinations can be optimally predictable (e.g., more predictable than score combinations) and can be valid regardless of the underlying distribution.

In some non-limiting examples, a data model within the pair of data models (or both) can be scores, sets of scores, score-based models, and the like. In some non-limiting examples, the use of scores can be a useful for establishing specific criteria such as an expected gain for including a stock in a group, a specific cut-off point for a biomarkers, and the like. As described above, rank combinations are optimally predictable (e.g., more predictable than score combinations) and are valid regardless of the underlying distribution, but such combinations are potentially less powerful than score combinations (e.g., due to the loss of distributional information and the reduced effect of valid outliers—outliers in the correct direction/direction desired). Thus, score fusions, can, in some cases, provide predictive rules (e.g., absolute thresholds) for future samples in ways that rank fusions cannot. As shown, these processes are valid for score and rank combinations and it is possible to recognize the region in which score combinations become less predictable.

In some non-limiting examples, a data model within the pair of data models (or both) can be deterministic or probabilistic models, which can produce deterministic or probabilistic outcome predictions (e.g., as an input to be considered for fusion).

In some non-limiting examples, a data model within the pair of data models (or both) can be from identical domains (e.g., two technical trading algorithms), two related domains (e.g., a technical and a fundamental trading algorithm), two relative unrelated domains (e.g., general stock market behavior, relative performance within a sector), or two apparently unrelated domains (e.g., a technical stock indicator and a metric based on car sales). In the apparently unrelated domain case, as described below, the processes and methods can be domain blind, and as such, these processes can combine any two models, intrinsically weighting them based on accuracy and diversity.

In some non-limiting examples, a data model within the pair of data models (or both) can be black box algorithms, such as models in which the inner workings are either not obvious to the creators of the algorithms (e.g., machine learning, AI, deep learning classifiers) or are deliberately loaded into a system (e.g., the computing device 102) in a language that cannot be deconstructed, e.g., compiled code, Haskell, thus ensuring trade secrets. In the latter case, the originator may have full knowledge of the method by which the algorithm works, but the user may not.

In some non-limiting examples, a data model within the pair of data models (or both) can be a fixed or an alterable model. For example, a data model can be hard-encoded and thus cannot be altered, hard-encoded such that they can only be altered by a vendor/manufacturer, encoded such that they can only be altered by someone having defined permissions (e.g., physical, software, biometric locks, and the like), or open such that they can be altered by any individual(s).

In some non-limiting examples, a data model within the pair of data models (or both) can be a general model or a proprietary model. For example, a data model can be either commercially available or proprietary algorithms, or a combination of these, as input into the processes and methods of the disclosure to be considered for fusion, regardless of whether such algorithms are generally known and available or whether they are proprietary. In particular, the suitable computing device can evaluate a pair of data models with only requiring specific outputs of the models, so as to maintain secrecy or propriety of some data models (e.g., the computing device can receive, indiscriminately without revealing the identity of the transmitter or sender, to allow multiple companies, individuals, and the like, to leverage company secret data sets, so as to allow greater collaboration in financial analysis (between two or more financial companies) such as stock selecting, drug development (between two or more pharmaceutical companies), or clinical trials (between two or more medical device, or drug companies).

In some non-limiting examples, a data model within the pair of data models (or both) can have a combined format (e.g., a combination of any of the data models described). For example, proprietary black box algorithms can be used with a series of different continuous outcome metrics on score and or rank fusions.

At 204, process 200 can include the suitable computing device selecting (and fusing) a pair of data models to generate a fused data model, a plurality of times. In some cases, random pairs of data models can be generated (or selected) from simulated data, and subsequently fused to construct a fused data model. This can be completed an appropriate number of times to generate a sufficient number of pairs of data models that are fused to construct a corresponding fused data model. In other cases, the pairs of data models can be selected based on an appropriate number of combinations, including all combinations, of pairs of data models from a pool of a plurality of data models. For example, in some cases, the pool of a plurality of data models can include individual data models each corresponding to a particular variable (e.g., a DNA marker) that relates to a first or second class (e.g., cancerous or non-cancerous). In particular, the pool of a plurality of data models can include specific groups such as, for example, 500,000 possible DNA makers as one group, and 10 clinical parameters (e.g., blood pressure) as another group, each group relating to the first or second class (e.g., cancerous or non-cancerous). Thus, in this case for example, each of the 500,000 possible DNA markers represent a data model, and each of the 10 other variables represent a data model, which can be combined in various ways, and numbers, to create a pool of pairs of data models that are fused. In some cases, after specific pairs of data models are selected, fusion can occur after or before steps 206, 208, 210, 212, (or others). As described below, fusion of the pair of data models, for the purposes of process 200 can occur in different ways. In some cases, fusion can include averaging the accuracies, the correlations, or both for the underlying data models that define the fused data model. In some cases, fusion of the data models can include normalizing the scores or ranks as appropriate for each data model for each class (e.g., the two classes). Then, for each class the scores from each of the two data models can be averaged, and the mean score of each pair can be calculated.

At 206, process 200 can include the suitable computing device determining a first accuracy for one of the data models within a pair of data models, and a second accuracy for the other of the data models within the pair of data models. In some cases, this determination can occur prior to fusion of the pair of data models that generates a fused data model. Determination of the accuracy for each data model of the pair of data models, can occur for all pairs of data models (e.g., those selected at 204 of process 200). In some cases, determining the accuracies of the data model can include determining the area under the receiver operating characteristic curve (“AUROC”) for the particular data model.

In some cases, assuming that each pair of data models are representative of at least a first class and a second class (or other classes), determining the first accuracy of the first data model can include determining the accuracy of distinguishing the first class or the second class (or others) based on the true assignment of the first class or the second class (or others) using the first data model. Similarly, determining the second accuracy of the second data model can include determining the accuracy of distinguishing the first class or the second class (or others) based on the true assignment of the first class or the second class (or others) using the second data model. In some configurations, the first (and second) accuracy can, as described below (e.g., in the examples), include averaging for the first data model (and the second data model) the accuracy of the distinguishing the first class and the accuracy of distinguishing the second class, based on the true assignment of these classes. For the continuous case, as described below, the accuracies for each of the first and second data models is the correlation with the truth (of the classes).

In some non-limiting examples, determining the first accuracy and the second accuracy of each pair of data models can include determining whether the first accuracy or the second accuracy is greater (e.g., better), which can then be indicated, stored, defined, etc., as the maximum accuracy of the pair of data models. Additionally, in some cases, after fusion of the pair of data models to create a fused data model, the accuracy of the fused data model can also be determined (e.g., using similar methods as how the first accuracy of the first data model, and the second accuracy of the second data model are determined). Then, in some cases, the change in accuracy between the accuracy of the fused data model and the maximum accuracy (e.g., the better of the two underlying data models) can be determined. This process can be completed for each pair of data models, and can be stored as appropriate (e.g., in a computer readable memory).

At 208, process 200 can include the suitable computing device determining a first correlation and a second correlation for each pair of data models. The determined correlations can be the within class correlation, for each class, between a pair of data models. For example, as described above, all the data models (e.g., those selected as 204 of process 200) are representative of both a first class and a second class. In other words, each data model is capable of classifying observations (e.g., a subject, and the like) into the first class and the second class. Thus, the first correlation can be the correlation between the pair of data models for the first class, and the second correlation can be the correlation between the pair of data models for the second class. In some cases, such as when desired to have minimal confounding dependence on global correlation, the first and second correlations for a pair of data models can be averaged. This determination of the first and second correlations for pair of data models for each class, can occur for all pairs of data models (e.g., those selected at 204 of process 200). In some cases, determining the first and second correlations can include determining the Pearson or Spearman Rank correlation for each pair of data models for each class.

In some non-limiting examples, the accuracy determination such as quality of outcomes (that can be used for a recommendation for or against fusion) can be assessed using a discrete metric, such as the area under the receiver operating characteristic (“AUROC” or “AUC”) curve, or a continuous metric, such as the Spearman Rank Correlation with is an objective Truth. There are a wide variety of potential metrics to characterize a discrete outcome or a characteristic of a classifier. As such, at least to the extent such metrics can be adapted to consideration as AUCs, the fusion approaches described here will be valid. It is also noted that, for any metric related to outcome quality, the methods and processes in the present disclosure can be used to determine whether fusions follow the same rules. Examples of metrics of interest include, but are not limited to, AUC, PPV (positive predictive value), NPV (negative predictive value), FNR (false negative rate), FPR (false positive rate), Gain, Loss, high/low values, relative risk, and the like. In some cases, metrics can be designed to optimize either high or low values of these. There are a wide variety of potential metrics to characterize a continuous outcome or the characteristic of a continuous outcome, some of which can include Spearman Correlation, Pearson Correlation, Kendall's Tau, Rasch Analysis, and the like.

In some configurations, the orientation of the models needs to be the same (e.g., in the same direction), prior to fusion of the pair of data models. For example, an orientation of one of the data models within the pair can be determined, and the orientation of the other of the data models within the pair can be determined. Then, based on, for example, the correlations of each model (e.g., if for example, the correlation with the target is positive for one data model within the pair, but negative for the other data model within the pair), each data model can be oriented in the same way. Then, once oriented properly, both data models can be fused to create a fused data model. In some cases, to orient the models appropriately includes normalizing values of each of the data models.

At 210, process 200 can include the suitable computing device determining a plurality of correlation groups. The plurality of correlation groups each have a uniform interval of correlation, across the entire span of correlation (e.g., from −1 to 1). For example, a number of “slices” or correlation groups (or intervals) can be determined, which separates the entire span of correlation into a distinct number of correlation intervals. As a more specific example, if the number of correlation groups is determined to be 20, then each correlation group would have a correlation interval of 0.1. As described below, there is an inherent trade-off between system speed and resolution, based on the selection of the number of correlation groups. For example, generally, the higher the number of correlation groups (e.g., 50, each having a 0.04 correlation interval) the higher the resolution of the boundary that separates good fusions from bad fusions. Thus, increasing the number of correlation groups can increase the resolution, which may increase the accuracy of the prediction (or recommendation). However, increasing the number of correlation groups increases the computational resources and thus decreases the overall speed of the recommendation. Thus, in some non-limiting examples, the suitable computing device can receive a user input (e.g., from a user interacting with the computing device) that is indicative of a selection of the number of correlation groups, the correlation interval for each group, the desired processing time, the desired accuracy or resolution, that can be used to determine (or select) the number of correlation groups and their correlation intervals.

At 212, process 200 can include the suitable computing device sorting each pair of data models (or fused data model) within one of the correlation groups. For example, the first and second correlations determined above, can be utilized to sort the specific pair of data models within a correlation group (having a correlation interval). For example, suppose the number of correlation groups is 20 (each having a correlation interval of 0.1) and the average of the first and second correlation is −0.85, then the computing device would sort this pair of data models (or fused data model) into the correlation group that spans (−0.9-0.8). This sorting of the pairs of data models (or fused data models) can occur for all pairs of data models (e.g., those selected at 204 of process 200).

At 214, process 200 can include the suitable computing device plotting (or associating), for each correlation group, the accuracies (e.g., the first and second accuracies) between each of the underlying data models within a pair of data models (or fused data model). For example, in some cases, one axis can be the accuracy of one data model (e.g., one indexed value within a pair, such as “0”) within a given pair, and the other axis can be the accuracy of the other data model (e.g., another indexed value within a pair, such as “1”) within the given pair. Thus, in some cases, for each correlation group, pairs of data models (which can be represented each as a fused data model) can be plotted using the accuracies of each underlying data model within the pair. It is appreciated that, in some cases, plotting need not be completed, and rather these can simply be an association of the pairs of data models. In some non-limiting examples, for each correlation group, data model pairs that have accuracies of less than 0.5 can be removed (e.g., the accuracies truncated at 0.5, or the accuracy interval being between 0.5 and 1). Alternatively, models having AUC-defined accuracy of less than 0.5 can be inverted.

At 216, process 200 can include the suitable computing device, for each correlation group, generating a curve. For example, the curve can be generated in many different ways. For example, the curve can be a locally weighted scatterplot smoothing (“LOWESS”) curve, or can be created using a machine learning model, and the like. Each curve, for each correlation group, can define a first region and a second region. The first region can be defined as the interior region bounded by the curve, whereas the second region can be defined as the exterior region that is not bound within the curve. In some cases, after the curve is generated, the curve can be extended (or manipulated) so as to more appropriately define these regions. For example, the curve can be extended to intersect one of the accuracy axes (e.g., −0.5), and in some cases, a portion left of this intersection point can be included in the first region.

In some configurations, the curves can be generated, for each correlation slice (or interval), using the systems and methods above, by only using fused pairs of data models as points that have a change in accuracy that is greater than 0 (e.g., positive). This can, for example, provide a higher certainty (or confidence) for recommending fusion of a pair of data models to be evaluated later.

At 218, process 200 can include the suitable computing device storing each curve for each correlation group. For example, this can include storing the coordinates of the curve, a function defined by the curve, an association between the specific curve and the correlation interval, data indicative of a curve (e.g., an accuracy range), and the like. This data can be stored in a lookup table for easy recall or comparisons of the data. In some cases, the data can be stored in the memory of the suitable computing device (or other computing device, such as the server 106, the server 108).

In some non-limiting examples, for each correlation slice, the generated curves can define accuracy thresholds (e.g., accuracy ranges) that depend on one (or both) of the determined first and second accuracies. For example, for each correlation slice, there can be “areas” that correspond to allowable accuracy ranges (e.g., where fusion is recommended). In other words, depending on the desired resolution, holding one accuracy value relatively constant (e.g., a substantially small accuracy interval) can provide an allowable accuracy range to be used for the other accuracy (e.g., that the other accuracy must be within for a recommendation for fusion), and vice versa. These ranges, intervals, etc., can be appropriately stored, such as in a look-up table in a computer readable medium so that these values are concrete (e.g., fixed), easily recalled, and easily comparable. As a concrete example, suppose for a specific correlation slice, that the generated curve defines, a first accuracy of 0.5, and that the second accuracy must be within and accuracy range of 0.6-0.8 for a recommendation of fusion. These data points, and ranges, can be stored for various resolutions, for each correlation slice. For example, corresponding accuracy ranges, based on the generated curves, can be saved for every 0.01 interval in accuracy (e.g., for each of the two accuracies of the data models). In some cases, the system (e.g., the computing device) can select a particular resolution, accuracy, etc., (e.g., based on a user input), that can correspond to the accuracy interval (e.g., 0.05, 0.01, 0.001, etc.).

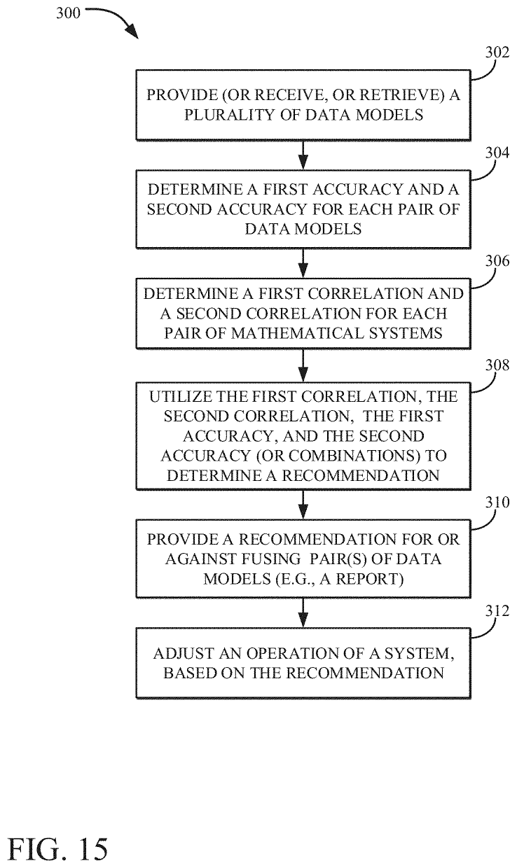

FIG. 15 shows a flowchart of a process 300 for evaluating fusion of pairs of data models. In some configurations, process 300 can be implemented entirely (or partially) on a suitable computing device (or devices), such as the computing devices 102, 104, server 106, or generally a computing system (e.g., the computing system 100).

At 302, process 300 can include the suitable computing device providing (receiving, or retrieving, such as from memory) a plurality of data models to the computing system, or computing device. In some cases, the plurality of data models can include only a single pair of data models. In other cases, the plurality of data models can include a pool of data models

At 304, process 300 can include the suitable computing device determining a first accuracy for one of the data models within a pair of data models, and a second accuracy for the other of the data models within the pair of data models (which can be similar to 206 of process 200). If, for example, there are many pairs of data models to be evaluated, this determination of the accuracy for each data model of the pair of data models, can occur for all pairs of data models (e.g., those received at 302 of process 300).

In some non-limiting examples, as described below, a geometric method (process, or framework) can be utilized to evaluate for or against fusion of one or more pairs of data models. For example, determining a first accuracy for one of the data models within the pair of data models and a second accuracy for the other of the data models within the pair of data models can include first generating a permutahedron (or permutohedron) based on the number of observations (or data points) within each data model. In some cases, each data model within the pair should have the same number of observations, where the number of observations can be either odd or even. The number of observations determines the number of dimensions for the permutahedron (e.g., for 10 observations the permutahedron is an S10 with a 9 dimensional hyperstrucutre embedded in a 10 dimensional space, for 9 observations the permutahedron is a S9 with a 8 dimensional hyperstrucutre embedded in a 9 dimensional space, and so on).

After generation of the permutahedron, different locations (or points) on the permutahedron can be identified (or determined). For example, the origin of the permutahedron (e.g., located at the barycenter of the permutahedron) can be determined after the generation of the permutahedron. Also, a target point (or reference point) on the permutahedron can be selected (or determined), such as a pole of the permutahedron. Then, a point for each data model of the given pair of data models can be determined on the permutahedron (e.g., the point being the vector coordinates of each ranking system on the permutahedron). Then, angles between these points of the permutahedron can be determined, which effectively correspond to the accuracies and correlation of the underlying data models. For example, a first angle defined between the first point on the permutahedron determined from the first data model, the origin of the permutahedron, and the target point of the permutahedron, where the origin is defined as the vertex can be determined, which corresponds to the accuracy of the first data model of the pair of data models. Similarly, a second angle defined between the second point on the permutahedron determined from the second data model, the origin of the permutahedron, and the target point of the permutahedron (the same target point used to determine the first angle), where the origin is defined as the vertex can be determined, which corresponds to the accuracy of the second data model of the pair of data models.

At 306, process 300 can include the suitable computing device determining a first correlation and a second correlation for each pair of data models (which can be similar to 208 of process 200). The determined correlations can be the within class correlation, for each class, between a pair of data models. For example, all the data models (e.g., those received at 302 of process 300) are representative of both a first class and a second class. In other words, each data model is capable of classifying observations (e.g., subject, and the like) into the first class and the second class. Thus, for each data model pair, the first correlation can be the correlation between the pair of data models for the first class, and the second correlation can be the correlation between the pair of data models for the second class. As previously described, for a pair of data models, the first and second correlations can be averaged (or combined as appropriate) to yield a single correlation value for the pair. Similar to 306 of process 300, if there are many pairs of data models to be evaluated, this determination of the accuracy for each data model of the pair of data models, can occur for all pairs of data models (e.g., those received at 302 of process 300).

In some configurations, such as the in the geometric framework, as another example, some angles between these points of the permutahedron can also be used to determine the correlation between the two models within a pair of models. For example, the angle defined by the point corresponding to the first data model, the point corresponding to the second data model, and the origin of the permutahedron (with the vertex being the origin of the permutahedron) can be used to determine (or can be associated with) a correlation between the two models. Similarly, an angle that is defined by the rotation around an axis that separates the origin from the target point, where the rotation distance is the distance separating the point representing the first data model from the point representing the second data model, can be used to determine or effectively corresponds to the correlation between the two data models. These angles (e.g. direct point-to-point angle, and rotation angle) correspond to two different interpretations of the correlation/diversity between the first data model and the second data model. The former includes the relative difference in data model accuracy (between the first data model and the second data model) in the correlation, whereas by considering only the rotation the latter formulation is independent of these differences in accuracy.

At 308, process 300 can include the suitable computing device, for each data model pair, utilizing the first correlation, the second correlation, the first accuracy, the second accuracy, or combinations of these to determine a recommendation for (or against) fusing each data model pair. For example, in some cases, these values can be used to determine (or retrieve, such as in a look-up table) a specific curve that relates to one or both of the correlations (e.g., the average correlation), which can be for example, the curve associated with a correlation interval that includes one or the both (e.g., such as an average correlation) of the two correlations. Then, the two accuracies for the pair of data models can be used to determine if the pair is located within the first region or the second region that is defined by the curve. If the pair is located in the first region (e.g., the internal area bounded by the curve), a recommendation for fusing the data model pair is stored, provided, and the like. If the pair is located in the second region, a recommendation against fusing the data model pair is stored, provided, and the like.

In some non-limiting examples, the order of evaluation of pairs of the data models can be implemented in a predetermined order, or an order based on specific operational conditions (e.g., based on clustering, correlation, and the like), or conditions selected by a user (e.g., via a user input). In some non-limiting examples, the use of multiple models can be a useful for selecting models against rigorous, predefined criteria. For example, sequential score fusions are the equivalent of fractional combinations of the individual models, where the fractional representation of each model in the final fused model is a direct function of its order in the fusion cascade. Thus, sequential rank fusions are not equivalent to summing methods (e.g., the Borda Counts or Kemeny consensus) because pairwise fusions rather than wholescale summations are conducted, evaluation is allowed at various independent stages, and the non-limiting examples of the disclosure can be used to seek improvement relative to an objective standard, while the others do not.

As another example, the correlations alone (and not the accuracies) can be used to provide a recommendation. For example, if one or both (or a combination) of the correlations are below a threshold correlation value or range, such as, for example, −0.9, then a recommendation for fusing the data model pair is stored, provided, and the like. Conversely, if one or both (or a combination) of the correlations are above the threshold correlation, then a recommendation against fusing the data model pair is stored, provided, and the like. In some cases, the suitable computing device can determine a difference between the first and second accuracies for the given data model pair. Then, one or both (or a combination) of the first and second correlations can be used to extract an accuracy difference threshold, to be used to evaluate against the accuracy difference. For example, as the correlation increases, the accuracy difference threshold decreases for a recommendation of fusing the given data model pair (e.g., the thickness of the curve bounded region—the first region). If the accuracy difference is below, or within the threshold range (e.g., ±5% in the accuracy between the two) then a recommendation for fusing the data model pair is stored, provided, and the like. Alternatively, if the accuracy difference is above or exceeds the threshold range for a given correlation, then a recommendation against fusing the data model pair is stored, provided, and the like.

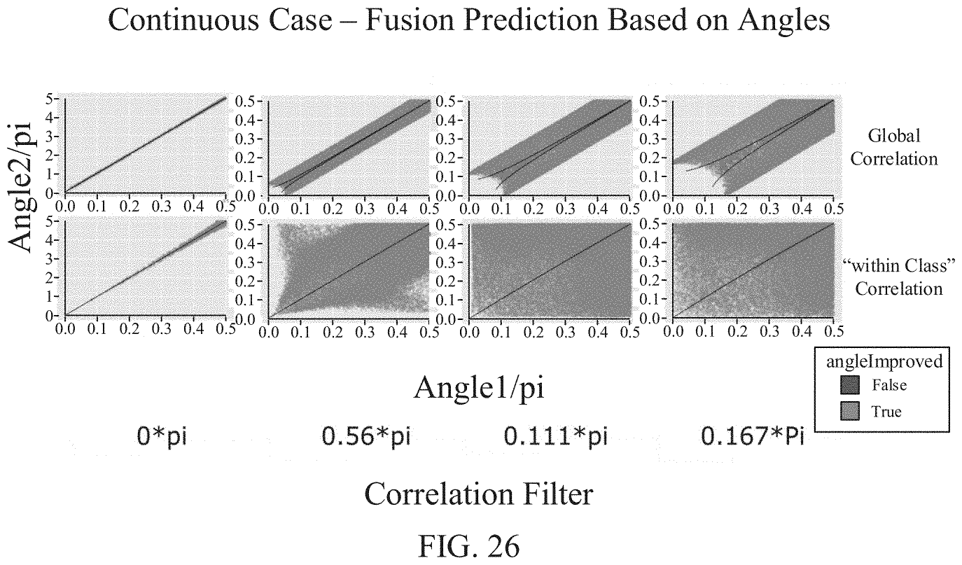

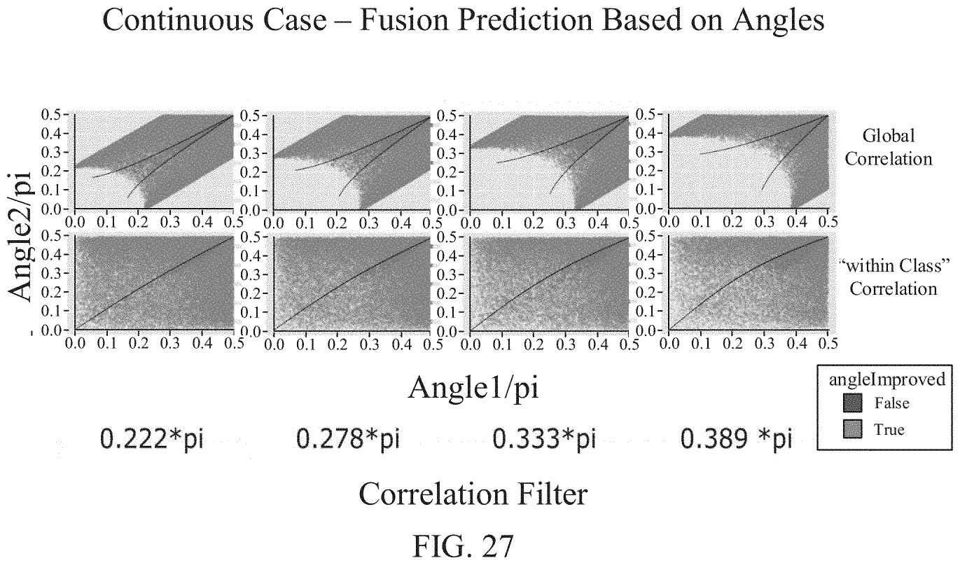

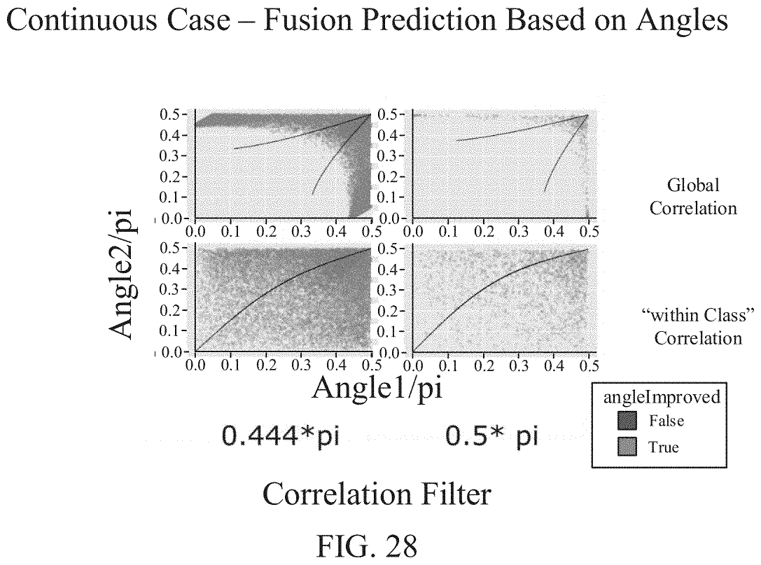

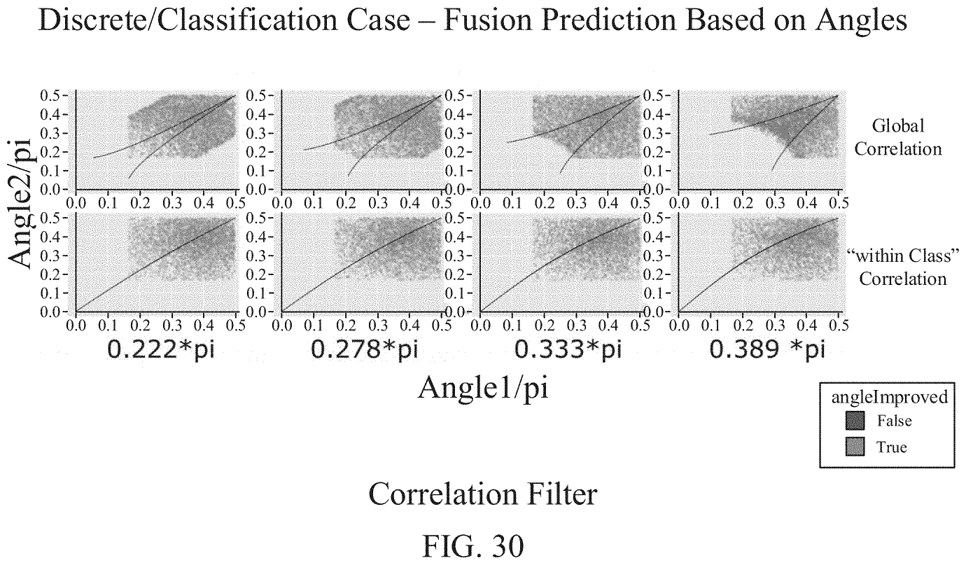

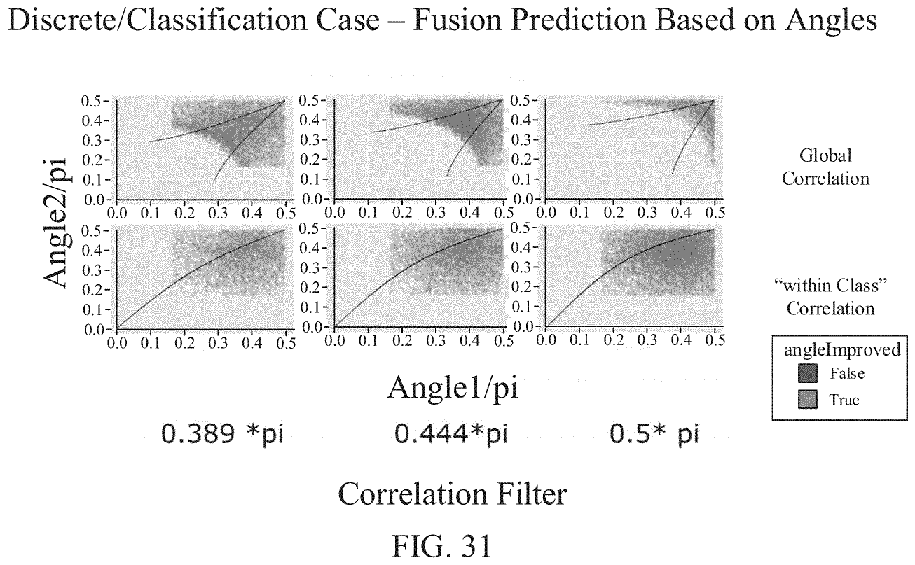

In some non-limiting examples, such as with the geometric example, particular angles (corresponding to the accuracies of the respective models within the pair of models, and corresponding to the correlation of the respective models within the pairs of models) can be used to determine, generate, or recall (e.g., from a look-up table) a corresponding evaluation curve (or threshold ranges, such as accuracy ranges) that corresponds to the angle (or correlation) and that can be used to evaluate whether or not it is recommended to fuse the data models. In some cases, the curve can be a theoretical curve (e.g., determined by . . . ), which is shown, for example, in FIGS. 26-31. Once the curve (or specific look-up table, or accuracy ranges that correspond to angle ranges) is selected, based on the correlation between the respective data models (e.g., or angle corresponding to the correlation) the accuracies of the respective data models (or corresponding angle) can be compared to the curve (or other data, such as the accuracy threshold ranges) to determine a recommendation for or against fusion, similar to the comparison of the non-geometric example above. In some configurations, the angles referred to above can be converted into correlations or accuracies, and vice versa, as appropriate.

At 310, process 300 can include the suitable computing device providing a recommendation for or against fusing a pair (or pairs) of data models. For example, a report can be generated and displayed (to a user) that details whether or not the data model should or should not be fused. In some cases, such as when there are many pairs of data models, the report can list each data model pair, with each having a recommendation for (or against fusing). In some cases, for fusing the pair of data models can be generated (and presented) as a text, other alphanumeric code, or simply a “1,” whereas against fusing the pair of data models can be generated (and presented) as a text, other alphanumeric code, or simply a “0.” In some cases, the accuracies of the data models within a pair of data models can be (or can be determined to be) substantially (e.g., deviating by less than 20%) similar, or in some cases exactly the same.

In some non-limiting examples, such as following a recommendation, a sequential fusion of two or more sets of scores (e.g., that are data models) can be completed (e.g., a pair of data models), followed by the conversion of the output of the sequential fusion (of the one or more such fusions) from scores to ranks. This can then be followed by the fusion of this ranked series (e.g., sequential fusion) with a least one other set of ranks. As an example, consider the data models A, B, C where A and B contain distribution/score, or data/information that is complementary and informative, whereas the score/distribution of C is closely correlated to ranks. Thus, in this one should consider a score combination of A and B, followed by a rank fusion with C. The actual efficacy of this fusion strategy, will, of course, be constrained by the properties denoted in the method.

At 312, process 300 can include the suitable computing device adjusting (or changing, such as improving) an operation of a system, based on the recommendation for or against fusing the pair (or pairs) of data models. In some non-limiting examples, the computing device itself can be the system that changes operation, or the computing device can instruct another system (e.g., the computing device 104) instructed to augment its operation, either of which are based on the recommendation. In some non-limiting examples, the system (as instructed) can fuse the data model pairs, currently, and as a default future option, which can increase accuracy for the system. In other non-limiting examples, the system (as instructed) can reject the fusion of data model pairs, currently, and a default future option, which can help the efficiency of the system (e.g., prevent unneeded calculations, or data acquisition thereby increasing computational efficiency, and increasing power efficiency, and reducing costs associated with data acquisition). In particular, the system can be adjusted by preventing (or mitigating) data acquisition from data used to construct one of the data models (e.g., the one with a lower accuracy or greater cost or burden for use).

In some non-limiting examples, different advantages can be realized by adjusting the operation of the system, based on the recommendation for or against fusion of the data models. For example, in some cases, computer technology generally can be improved by decreasing computational time for a computing device (e.g., the computing device 102). In particular, by enabling direct comparison of pairs of models, non-limiting examples of this disclosure collapse the need for examining all possible combinations of variables for all observations (which scales exponentially). Rather, a calculation (or calculations), that can include the accuracy and correlation determinations, can be evaluated by only needing to compare the calculations to a look up table or other threshold (which scales sub-exponentially, typically linearly). This provides a time advantage, which is critical in many domains, e.g., stocks, medical care.

Some non-limiting examples of the disclosure can allow a system (e.g., a computing system, a computing device, and the like) to increase the search space. For example, by enabling direct calculation of the potential effects of fusion with fully calculating options, non-limiting examples of the disclosure make it possible to inherently consider larger sets of potential combinations of models for applications per unit time, which again can improve computer technology generally, by better utilizing computational resources.

Some non-limiting examples of the disclosure can enable decision making without training. For example, decisions can be made regarding the possibility of fusing two or more models without requiring additional training sets. In particular, utility of combining markers or predictors from two unlinked datasets (in cases where the correlation can be estimated) can be decided.

Some non-limiting examples of the disclosure can enable decisions and actions without secondary intervention. For example, some non-limiting examples of the disclosure can be utilized to make decisions about the fusing of two or more models without requiring additional action from an outside source (e.g., human, machine). In other words, some non-limiting examples of the disclosure, such as when operating on specific conditions, can create autonomous decision making systems and devices. As one example, some non-limiting examples can recognize unanticipated emergency situations (e.g., behavior outside combined models) and begin notification and or remedy (a situation). As another example, in decision systems, such as for self-directed devices, vehicles, and the like (see also below).

Some non-limiting examples of the disclosure can provide a point of use (or care) improvement. For example, some non-limiting examples of the disclosure can be applicable to devices that are designed to be field applicable, such as point-of-care medical devices, cell phones, and the like, where the combination of ability to act autonomously, speed, reduced computational power, and the like as utility.

Some non-limiting examples of the disclosure can improve device speed and energy utilization (e.g., conserve energy, increase speed, and the like). In some cases, data transfer requirements for various devices (e.g., a computing device) can be reduced. A significant problem on some devices (e.g., cell phones) is the amount of power required for calculations and the power of the processors themselves. A major fraction of the energy usage (and time) results from the constant need to move data back and forth to allow computation. This problem increases exponentially as one wishes to leverage multiple models. Thus, by stripping a model down to accuracy and correlation for evaluation avoids requiring the computing device to transfer all of the data. For example, consider a dataset with 10,000 observations each with 10,000 variables with four significant figures—transferring the data requires 1,000,000,000,000 units, whereas transferring the rank order is ˜40000 units. The gain in energy efficiency—which increases battery life, potentially battery weight, operational time, and the like, would be raised to the number of factorial combinations of the number of models engaged, and would also include data transfer and computational time. The last may also be particularly critical as on-board/POC processor are typically less powerful than, for example, centralized or cloud-based devices.

Some non-limiting examples of the disclosure can assess the impact of variable fusions, so as to determine whether or not to include variables in a larger system or model, or whether to continue to collect a given data such as sensor data (e.g., a variable that is not useful may no longer needs to be collected). The efficiency of the system allows for the sequential and consistent testing of all relevant models with less than a full complement of data (e.g., consider N variables, then delete 1 variable at a time, 2 variables at a time, and the like). When and if the processes determine that a given variable is no longer useful (e.g., based on the recommendation against fusion) it can command a system (e.g., a suitable computing device) to no longer use the variable (e.g., prevent computation of the variable from acquired data, prevent acquisition of data that is used to determine the variable, and the like), it can switch to smaller models, it can actively disengage from collecting that variable and/or passively no longer collect it. This can save acquisition time and costs as well as potentially reducing over-fit errors for the primary models. Similarly, non-limiting examples of the disclosure can be set to terminate the use of an entire system, if it fails some user specified criteria (e.g., it is never used in the final model over a given time period).

Some non-limiting examples of the disclosure can provide a method for assessing (or deciding) downstream path for data analysis. Artificial intelligence often runs on specialized computer chips. Thus, consider a case where there are, for example, three specialty chips—C_{A}, C_{B}, and C_{C}—and you want to use C_{A} if condition A prevails, C_{B} if condition B prevails, and C_{C} if conditions C prevails. In this case, a series of systems that are able to identify/predict conditions A, B, and C are evaluated using the processes and methods of this disclosure to then route any follow-up analysis to the chip corresponding with the condition determined. Related configurations include the choice of which method, system, and the like to utilize for follow-up analysis (e.g., deciding between regression and projection based methods, such as LASSO and PLS).

Some non-limiting examples of the disclosure can enable single shot learning. For example, the use of the systems and methods of the disclosure are applicable to devices that are designed to build actionable input off single (or very limited training information). In general, humans can learn from a single instance, but machine learning models that require training are limited in their ability to do this. Here, some non-limiting examples take the input (e.g., scores, ranks) from two or more data models, and uses the systems and methods of the disclosure to output the best option in terms of fusing inputs or choosing not to.

Some non-limiting examples of the disclosure can enable primary model creation. For example, according to some non-limiting examples, models can be created without mathematically over-fitting or over-applying redundant information. For example, since a given data model can be as simple as a single measurement, non-limiting examples of the disclosure can be utilized to fuse many pairs of models (and subsequent pairs of models after an initial fusion) to build primary models. In particular, the output of the determination of fusions (and fusions, if determined so) can be used directly (e.g., as input back into evaluation and subsequent fusion in some cases, or in conjunction with other systems), directly (e.g., by being exported as an interpretable model, indirectly to induce an action (e.g., to determine a stock or set of stocks to buy), or indirectly to provide information (e.g., a rank order of stocks).

Some non-limiting examples of the disclosure can provide for the enabling and synthesizing of a human input(s). For example, systems and methods of the disclosure can fuse a human supplied input (e.g., a person's opinions of how objects should be ranked) with that of other humans and/or systems (e.g., output from machine learning algorithms).

Some non-limiting examples of the disclosure can include setting up the processes (and methods, such as implemented on a suitable computing device) such that when the processes choose between multiple models, it is set up to either record or not record the model's input (or lack thereof). Similarly, it can report or not report this usage. For example, reporting such information (e.g., to a server, such as the server 106, or other database or other computing device) can be used to improve subsequent work, to credit the party who's model (or data) are used, to back-check such as doing post-analyses when models fail. Alternatively, the user may wish not to capture or report model usage (e.g., to maintain confidentiality, trade secrets, and the like).

Some non-limiting examples of the disclosure can combine particular uses and formats described above, such as the combined use of the formatting options envisioned above. For example, systems and methods of the disclosure can use proprietary black box algorithms with a series of different continuous outcome metrics on score and or rank fusions.

Some non-limiting examples of the disclosure provide a highly flexible framework/platform that enables a broad series of specific applications, which can include two types of domains—those that are discipline specific (e.g., medicine, finance, and the like) and those that are cross-disciplinary (e.g., sensors, machine learning, computational efficiency, and the like).

Some non-limiting examples of the disclosure provide various Machine Learning applications, which can improve the ability to build models, through the selection of models, the use of the fusion decisions (e.g., outputs) to be used as subsequent inputs, and the selection of optimal paths for sequential fusions. As one example, some non-limiting examples can provide real-time and static testing of model applicability, which can be used to determine which data models/predictive algorithms (or models) should be included when multiple such tools are available, and which data sets (e.g., two different financial datasets that differ in the variables measured in that dataset). One can examine the accuracy and correlation structure between existing models so as to determine whether a given model can stand alone, can be beneficial in the context of a fusion, or should be disregarded in the context of a model. The method can, for example, be used to determine which models are functionally non-informative or mis-informative (e.g., do not add in the context of other models) and should be dropped (e.g., eliminated, prevented from utilizing calculations, prevented from acquiring data, and the like). The methods and systems can complete this with minimal or no test set data. Also, these methods and systems make it possible to determine whether there are algorithms that are more likely than others to be useful (e.g., consistently better accuracy in conditions of interest, consistently less correlated predictions with other models), and that this information can be used to prioritize such models (e.g., by prioritizing relevant data collection).

As a second example, some non-limiting examples of the disclosure can provide stand alone and integrated implementations. For example, systems and methods of this disclosure can stand alone “on top of” other algorithms or can be integrated either in parallel, in series, or directly into other novel machine learning algorithms. As a specific example, the output created through the use of the systems and methods can itself be used as an input into other, downstream applications and/or modeling pipeline (automated, semi-automated, or manual). This would include, for example, the use of these systems and methods as an essential component of novel machine learning algorithms.

As a third example, some non-limiting examples of the disclosure can provide a scheme for optimization of sequential fusions. As an example, it is possible to leverage the predefined decision tree that some non-limiting examples follow to determine whether it is possible to arrive at later steps in a set of sequential fusions with better fusion partners (e.g., less correlated models) using one set of fusions vs another, or using one set of models vs another. This can be scripted and embedded within a system to, for example, for any set of models, determine an optimal or near optimal path of sequential fusions, either by algorithmic approaches (e.g., a clustering-like approach with an appropriately weighted distance algorithm, or by a simulation of all possible series of fusions).

Some non-limiting examples of the disclosure provide improvements in computational efficiency and computational resource allocation (e.g., conducting certain types of calculations more efficiently) for computing devices, computing systems, and the like. Some non-limiting examples allow for the ability to act rapidly and efficiently in situations that are not amenable to training sets, absolute calculations, structural equation modeling, and the like. As one example, non-limiting examples of the disclosure can increase the computational speed of, for example, computing systems by turning combinatorics into use of a look-up table or analysis on a geometrically constrained surface, and the like, which can speed up calculations. In addition, this configuration hastens the analysis of some problems by eliminating some calculations (e.g., showing that some models are non-informative). As a second example, non-limiting examples of the disclosure can decrease computational complexity/narrow the search space by showing that certain models can be ignored. For example, models that have low accuracy cannot add to high accuracy models without showing negative correlations, low N models cannot increase the certainty of high N models. Thus, the method can be used to reduce the number of models that can be considered for fusions. Notably this also makes the analysis faster, less energy intensive, and the like. As a third example, non-limiting examples of the disclosure can increase the potential computational search space (that would otherwise be limited due to computational constraints) by simplifying the calculations necessary for determining (or recommending) for or against fusing data models. For example, non-limiting examples of the disclosure can enable the user to search a larger search space from the increase in computational efficiency in scenarios where the space that can be searched is functionally limiting. This would include, for example, any scenario where the possibilities need to be or can be advantageously considered in groups. For example, a group of N objects considered independently scales linearly with N, whereas considering the group in sets of three without replacement scales as N*(N−1)*(N−2), a system considering all orderings scales as N!, and the like. This type of problem is relevant in, for example, assembling a stock portfolio.

As a fourth example, non-limiting examples of the disclosure can decrease system requirements for specific computing devices (e.g., low power computing devices). For example, non-limiting examples of the disclosure have utility in enabling computational efficiency in scenarios where the lower computational power of the computational device is functionally limiting. This would include both user-enabled systems such as a mobile phone, but would also include user-independent systems such as embedded/implanted and stand-alone sensors that need to act (e.g., send a signal) on an event occurring, and would need to decide which of multiple models to base this decision. In some cases, the device can be a mobile phone, an implanted health device, and the like.

As a fifth example, non-limiting examples of the disclosure can off-load computational requirements (e.g., enabling use on low power devices). Although non-limiting examples can enable usage on devices with low computational power, it is apparent that, if desired, the user can off-load calculations onto other devices (e.g., networked computers, web applications, and the like). As an example, Google searches are done on the web, not on a computer. In one non-limiting example, the invention off-loads (pushes) calculations that generally are non-informative to another system (potentially also using the non-limiting examples of the disclosure), along with instructions to push the models back if they hit a certain threshold (e.g., a predetermined accuracy or correlation).

Some non-limiting examples of the disclosure provide improvements in sensors systems, signal processing, and reactive systems. It is not surprising that the interpretation of data from multiple sensors is a classic case in information fusion. Sensors can range for items such as detectors (e.g., motion, fire, smoke), but also include devices such as hearing aids that clarify signals for direct consumption, or implantable medical monitors. Whether the information to be fused crosses time, distance, information type (biometrics, motion) there are a near infinite number of possible combinations, and potentially hundreds of models as well as multiple layers of potential models. As such, it is generally impossible to provide adequate training sets. Some non-limiting examples can address these by either embedding the choice based on the method (use methods A, B, C if method A threshold crossed, for example) or allow the sensor array to learn and adapt. In one case, multiple models can be used to identify noise so as to optimally factor out its contribution. One system could seek noise based on size, another on frequency, another on duration and the like. As one example, non-limiting examples of the disclosure can improve the interpretation of sensor data from a sensor array (or a system having multiple sensors) by improving modeling across the entire range of the responses of one or more sensors (or one or more situations, e.g., the same sensor across time) so as to detect and accurately capture weaker, rarer, or more complex signals by both amplifying signal and improving pre-acquisition understanding of the signal itself. In some cases, different sensors independently rank detected movement (human, animal, wind, electrical artifact, and the like), and these sensor inputs are subjected to the methods/processes of the disclosure to determine the most likely cause, and the ranked list leads to an action (e.g., likely human then sound alarm, likely wind then ignore). Many different properties of interest can be monitored (e.g., increasing positive predictive value, decreasing false negatives, and the like), and that these are the primarily properties of the inputs for the systems and methods of the disclosure. Thus, these systems and methods can enable improved modeling across the entire range of the predictions of one or more models (or one or more situations, e.g, the same model under changing environmental conditions) so as to detect and accurately capture weaker, rarer, or more complex signals by both amplifying signal and improving pre-acquisition understanding of the signal itself.

As a second example, some non-limiting examples of the disclosure can improve the management of (meta) sensor arrays (e.g., by automating the management of meta-sensor arrays). For example, some non-limiting examples can determine whether the potential addition/activation of different sensors can add information or conversely, where deletion or inactivation of a sensor either reduces resource usage at minimal/acceptable cost or improves signal fidelity by reducing, for example, false negatives. Some non-limiting examples enable this type of adaption to occur either statically (e.g., the system adds/subtracts a sensor and produces a different series of models), or adaptively (e.g., in the presence of signal A, adapts and uses configuration of sensors A′, but in the presence of signal B, adapts and uses confirmation of sensors B′; adding or inactivating sensors as needed). System gains can be reflected in output, scanning speed, energy utilization, sensitivity, specificity, and the like. Some non-limiting examples also allow for monitoring outputs (e.g., of the processes) and use this information to manage the array. In some confirmations, sensors can be deactivated when some non-limiting examples determine that one sensor underperforms another by a sufficient enough that the correlation between such signals (e.g., sensor outputs) indicate that it can never (or not under circumstances deemed relevant) increase sensor accuracy/utility.

As a third example, some non-limiting examples can improve the analysis of data from sensor arrays (e.g., adaptively adjust sensor data acquisition, variable calculations, and the like). Some non-limiting examples can optimally identify potential adaptions to the interpretation of sensor signals (e.g., altering the tradeoffs between false negatives and false positives that would best integrate with existing sensors to improve target acquisition). Some non-limiting examples enable this type of adaption to occur either statically (e.g., the system resets and produces a different series of models), or adaptively (e.g., in the presence of signal A, adapts and uses configuration A′, but in the presence of signal B, adapts and uses confirmation B′). Gains can be reflected in output, scanning speed, energy utilization, sensitivity, specificity, and the like. This adaptation can also be deliberately triggered or activated by an influence external to the sensor/sensor array (e.g., an implanted medical sensor could be activated by a physician based on patient need, a user input, and the like).

As a fourth example, some non-limiting examples can provide read and react sensor systems, in which actions are initiated based on sensor/monitor, output/analysis, and the like. As one non-limiting example, the system senses a physiological/biological/clinical change of interest and automatically delivers medication/activates implanted device (e.g., neural, cardiac, and the like) and notifies medical staff (e.g., transmits a notification, alarm, and the like to a computing device). In another, the invention integrates human input and biochemical data to monitor and treat a patient. In another non-limiting example, building monitors react to a series of model inputs and warn inhabitants. In another non-limiting example, such a system can be used to help control an exoskeleton, or artificial limb. Multiple inputs can enable a finer level or control and a greater chance to recognize issues at what would normally be considered a subthreshold level while reducing false positives.

Some non-limiting examples provide improved time series and prediction systems (e.g., treat assessment over time). Threats can fall into multiple categories, including but not limited to personal issues (e.g., medical), financial issues (e.g., market crashes, loss in value), to larger scale events such as weather/storms/tornados and terrorist attacks. Standard prediction systems cannot readily integrate models (e.g., lack of training data, for example) and it is difficult or impossible to tell which models to believe (e.g., consider spaghetti plots for hurricane tracking). In contrast some non-limiting examples allow tracking risk with constant updating as the system can switch between individual models and fused systems, always optimizing given parameters (e.g., starting accuracies, dynamic shifts, and the like) and because of the systems/processes, calculations can be very fast and continue to shift which models as appropriate. In the case of a yes/no prediction, it is analogous to the classification problems. Some non-limiting examples can also be used on a single time point data to obtain an instantaneous risk, which can be logged for the series (e.g., over time) to determine if risks are increasing or decreasing over time. Output from the systems and methods can be customized (e.g., reporting, reacting, altering upstream or downstream models, and the like).

Some non-limiting examples provide improved resource allocation (e.g., optimizing allocation of resources). In most situations, the resources needed to address ongoing needs or situations are either finite, associated with costs, or both. In addition, fluidly changing situations require rapidly changing resource allocation. Predicting loads and/or predicting how resources will attenuate demand facilitates resolving these challenges. This problem is made more difficult by rapidly changing situations and the fact that those situations are often not available as training data. This is a broad problem that occurs in many areas. Some scenarios include the active management of day to day flow within city/business, and the like during events planned (e.g., concerts), short common occurrences (e.g., heavy traffic, accidents), emergent events (natural disasters, fires, terrorist), includes event planning, urban planning. Another example is hospital management: Patient flow, optimal patient care with limited staff, surgical flow, ER flow, supply chain. Businesses have similar issues, customer flow and optimizing customer service, adaptive supply chains. Another example is more technical, optimizing computer processing across multiple jobs, cores, and the like. Planning can be in real-time or done in advance, modeled as cost functions or as absolute needs. Optimization can differ for different situations, such as zero sum games with tasks having similar or different priorities, ensuring sufficient resources to carry out a task, optimal balancing of resources. The systems and methods allow for any number of models to be considered.

Some non-limiting examples provide for improvements to disciplines such as medicine, finance, autonomous vehicles, and the like. Clinical care and hospital operations represent a growing opportunity to leverage large data sets, modern informatics, machine learning approaches—and for non-limiting examples of this disclosure to provide improvements to this area. Past approaches to care have often focused on single markers (e.g., glucose for diabetes) and clinical expertise, but more powerful imaging approaches and laboratory assays have opened the gates to improving care at the individual level. At the same time, cost containment, efficiency, and cost effective delivery of care has become of increasing importance. There are at least two major factors that limit the utility of large datasets in clinical care. One is that decisions that impact care must often be made quickly, without the ability to allot time to systematically train models. Another issue is that, for many situations, there are too few examples and too many variables to deal with the multiple comparisons problem. For one reason, patients are individuals and, in many cases, have differing backgrounds, comorbidities, and the like. These factors limit what can be done to use high powered informatics approaches for clinical care. The systems and methods described herein expand these limits in three ways: (i) by speeding up certain types of calculations, the method enables combinations to be considered that would previously have been computationally impossible or impractical; (ii) by embedding decision making systems, it facilitates automated care systems, such as implantable devices. Finally, (iii) enabling the integration of models for which training set data either does not exist or would be impractical to obtain or to use.

As an example, some non-limiting examples can improve personalized medicine (e.g., optimizing personalized delivery of medical care. Systems and methods allow for the fusing of multiple models to improve the care of an individual. As one non-limiting example, one could consider diagnosis based on laboratory tests, questionnaires, genetics, anthropometric variables, and the like. As another, one can consider choices of chemotherapy regimen based on an individual's genome, their tumor's genetic changes, lab tests, and current population level statistics given their clinical description. These systems and methods can help determine which models should be factored in, and can make this choice in real time, without additional training data. Both the general case and pre-specified conditions can be studied. In general, these systems and methods potentially improve the delivery of personalized medicine by determining which models and/or modalities are most useful alone, which should be combined for optimal power, and which should be dropped either overall or for specific individuals.

As another example, some non-limiting examples can provide (and optimize) real-time decisions (e.g., ICU, Trauma, Personalized Care, and the like). Medicine frequently requires rapid decisions in the context of multiple competing datasets in complex, life-threatening situations. These situations include, but are not limited to, management of acute trauma (e.g., car accidents, military/battlefield situations) and intensive care units. Trauma cases may be under care in a hospital or field setting. The situations are complicated when there are multiple potential courses of actions (e.g., drug regimens, which problem to address first, and the like), and potentially further complicated by personalized medicine aspects. These systems and methods allow simultaneous, real-time consideration of multiple courses of action without requiring large training sets. In addition, one can use these systems and methods to simultaneously account for individual specific risks (e.g., personalized medicine). These systems and methods are capable of deciding and potentially initiating the optimal course of action in the face of contradictory predictors.

As another example, some non-limiting examples can provide improvements to mobile care, or off-site care. These systems and methods can be used to optimize off-site health care delivery. Medicine increasingly relies on off-site care, which may be delivered by skilled nurses/caregivers but in some cases, may be delivered by a family caregiver. In any case, these systems and methods can be used to integrate information on the patients background (e.g., diagnoses, most recent labs, genetics, and the like, with a current situation, (e.g., patient is eating, not able to move and the like) so as to increase for example, the quality of outcomes, optimally decide who should be brought in either to a doctor's office or an emergency room, or determine what care should be delivered on site. Such decision-making can be automated, reducing time and costs

As another example, some non-limiting examples can provide improvements for implantable devices. These systems and methods can be used to increase the capacity of implantable devices. Implantable devices offer the ability to mimic natural delivery of key endogenous chemicals such as insulin or to optimally deliver exogenous agents such as chemotherapy agents. These systems and methods makes it possible to program such a device to anticipate needs and/or to optimally and rapidly adjust to changing conditions without exogenous intervention. The implantable device can use models based solely on deliberately encoded models or may adapt such models in real time by aggregating sensor data from other implanted or exogenous sensors. In a related non-limiting example, these systems and methods can be used to track sensors over time and prioritize sensors that are receiving altered signals over time (or sensors whose alterations are driving models changing the current status/reaction). Such implanted devices can be programmed to, for example, use multiple models to decide when exogenous interventions are needed.

As another example, some non-limiting examples can provide improvements for trans omics analysis (e.g., better coordination). There is a growing ability to collect data on multiple readouts in clinical settings. This may include, but not be limited to, genomics, transcriptomics, proteomics, metabolomics, microbiota, and the like. This data offers the potential to improve individual care, to facilitate and to strengthen clinical trials, and to improve drug development. A critical limit, however, is that the multiple comparison problem that substantially limits the ability to work with these large datasets. These systems and methods enable such analyses to be made by largely eliminating the multiple comparison problem through the ability to integrate a series of models made using these complex datasets individually.

As another example, some non-limiting examples can provide improvements for clinical decision making/modeling. These systems and methods can be used to optimize clinical decision making. These systems and methods can optimally manage the day to day health of an individual, and can, for example, minimize returns to a hospital setting within 30 days so as to reduce costs to the hospital. These systems and methods facilitate the overall care of a patient and improve clinical decision making by leveraging the growing ability to collect large amounts of data on multiple readouts in both non-clinical and clinical settings (clinical information, test results, local information), by using data from a hospital or remote setting, with caregivers or automated sensors, and the like. This data offers the potential to improve individual care and minimize health care costs. A critical limit, however, is that the multiple comparison problem substantially limits the ability to work with these large datasets. These systems and methods enable such analyses to be made by largely eliminating the multiple comparison problem through the ability to integrate a series of models made using these complex datasets individually. These systems and methods can potentially act at any and all levels (e.g., prognostic, diagnostic, treatment, and the like) under cases where there are too few historical patients for large cross-validation requirements, (e.g., surgery, complications). These systems and methods are also capable of optimizing the ability to recognize at risk patients (e.g., complications, disease course, readmissions). In some cases, systems and methods can flag likely (hospital) (re-)admissions and times, so as to minimize such (re-)-admissions, e.g., through the use of visiting nurses.

As another example, some non-limiting examples can provide improvements to hospital resource allocation (e.g., optimize allocation of resources).

As another example, some non-limiting examples can provide improvements for clinical trials (e.g., optimize clinical trials). In some cases, a user can seek to optimize power at the level of enrollment by ranking potential subjects according to criteria of interest. In one case, these systems and methods could be used to maximize enrollment of individuals who will go on to develop a given disease in a period of time, so as to maximize the power in a prevention trial. This reduces costs and increases the chance of a successful trial by reducing statistical noise. In this case the input models would seek to predict which individuals will reach an endpoint and output a ranked list of who should be enrolled to improve power. In a second case, one can seek to improve power in a trial (and/or reduce costs) by using the systems and methods to determine in advance whether combining multiple test (e.g., multiple biomarkers) is likely to increase power to detect a phenotype of interest. In this case the inputs would be biomarkers known or suspected to inform about the condition, and the outputs would be which biomarkers to include.

As another example, some non-limiting examples can provide improvements for biomarker development (and non-medical predictors). The systems and methods can be used to optimize biomarker development by determining the likely interaction of two biomarkers arising at different times. As an example, consider one accepted biomarker and the potential of a new biomarker. The framework can be used to determine whether it is likely the first biomarker will prove superior to the second (and any possible combination) and thus marker B should be dropped, minimizing losses), the second will add power if used in conjunction with the first (in which case the first should be licensed and the second patented/pursued), or the second will supplant the first (in which case the second should be pursued/patented and the first licensing fees can be bypassed). These different results have clear financial and health implications (go/no go, requirements for a second trial, licensing requirements, and the like). In another non-limiting example, one can use these systems and methods to discover synergistic biomarkers. Specifically, one can leverage the power of the technique to search a different “space” biologically, with assurance that only biological and not mathematical over-fitting will occur.

As another example, some non-limiting examples can provide improvements for mainstream finance and investing. Financial markets are well-characterized targets for modern informatics and machine learning approaches. Indeed, technical trading relies on modeling and/or pattern recognition. Current approaches gain their power from the existence of large, high quality data sets that can be used as training. This reliance, however, also defines the limits of what can be done to use high powered informatics approaches for trading. The method described here expands these limits in three ways: (i) by speeding up certain types of calculations, the method enables combinations to be considered that would previously have been computationally impossible or impractical; (ii) by enabling the determination of the potential gains in fusing data sets, the method provides options in terms of identifying additional models that should be used or created, identifying non-informative models, (iii) enabling the use of models for which training set data either does not exist or would be impractical to obtain or to use. When the options for identifying value and potential risk reduction complements within marketplace cross time, distance (e.g., different markets), instrument type (stock, bond, fund, future, option, and the like), domain (e.g., industry sector) there are a near infinite number of possible combinations, and potentially hundreds of models as well as multiple layers of potential models. As such, it is impossible to provide adequate training sets, comprehensive modeling is too slow and would face too large a multiple comparison problem. Options include either embedding the choice based on the method (use methods A, B, C if method A threshold crossed, for example) or allow the system to learn and adapt. In either case, the method clearly has multiple possible implementations.

As another example, some non-limiting examples can provide improvements for the optimized prediction of the relative or absolute future value of one or more specific assets. Multiple models can be fused to improve the relative pricing between two assets or the absolute price of an asset, and both can be done under varying conditions of interest, either simultaneously or separately. Such questions can be successfully addressed even in situations such as real estate, initial public offerings (IPOs), political or economic instability, and the like, where past experience is of little direct value and one cannot cross-validate the models. Both the general case and pre-specified conditions can be studied. The relative pricing between two or more assets can be used both to stabilize a portfolio or for arbitrage purposes The systems and methods enable improved modeling across the entire range of the predictions of one or more models (or one or more situations, e.g., the same model under changing market conditions) so as to detect and accurately capture weaker, rarer, or more complex signals by both amplifying signal and improving pre-acquisition understanding of the signal itself.

As another example, some non-limiting examples can provide improvements for the addition of complementary financial models. These systems and methods can optimally determine a potential model or models that would best integrate with an existing model to improve the prediction of future prices, market conditions, and the like. The method enables this type of adaption to occur either statically (e.g., the system adds a model and produces a different series of models), or adaptively (e.g., in the presence of signal A (e.g., a market decrease), adapts and uses configuration of model A′, but in the presence of signal B (e.g., high volume, market increasing), adapts and uses confirmation of models B′)—adding or inactivating models as needed. Gains can be reflected in output, computational speed, energy utilization, sensitivity, specificity, and the like. This adaptation can also be deliberately triggered or activated by an influence external to the modeling system, e.g., an unexpected real-world event that leads to expert opinion can be created as a model.

As another example, some non-limiting examples can provide improvements for managing and adapting multiple model systems (e.g., automating the management of multiple model arrays). For example, these systems and methods can determine whether the potential addition/activation of different models can add information or conversely, where deletion or removal of a model either reduces resource usage at minimal/acceptable cost or improves signal fidelity by reducing, for example, false negatives. The method enables this type of adaption to occur either statically (e.g., the system adds/subtracts a model and produces a different series of models), or adaptively (e.g., in the presence of signal A, adapts and uses configuration of systems A′, but in the presence of signal B, adapts and uses confirmation of systems B′)—adding or inactivating models as needed. Gains can be reflected in output, scanning speed, energy utilization, sensitivity, specificity, and the like. These systems and methods can monitor the outputs and use this information to manage the array. These systems and methods can optimally identify potential elimination of models (e.g., altering the tradeoffs between false negatives and false positives that would best integrate with existing models to improve false positives or false negatives with regards to model outputs). In one case, models are deactivated when these systems and methods determine that one system underperforms another by a sufficient enough that the correlation between such signals indicate that it can never (or not under circumstances deemed relevant) increase overall model accuracy/utility. It is apparent that this adaptation can also be deliberately triggered or activated by an influence external to the modeling system (e.g., an unexpected real-world event that leads to expert opinion can be created as a model).

As another example, some non-limiting examples can provide improvements for portfolio management. These systems and methods described can be integrated for use in assembling a portfolio. For example, one can assess the relative future value of sets of related assets, buying the one expected to have a greater value while selling the one predicted to have lower value. One can choose higher and lower risk assets, one can determine whether assets are priced equivalently (e.g., have equivalent expected gains per cost of carrying the asset). One can use the method to either set up a series of portfolios having different objectives (e.g., minimize potential risk, maximize expected gain, and/or minimize volatility in a portfolio) or combine these in desired ratios. In other cases, these systems and methods can be utilized to automatically re-evaluate pairs of data models. For example, these methods for determining if (or if not) to fuse data models can occur after the system receives (e.g., from a user input) an indication that an underlying data model has changed. This way, if the system (e.g., using the methods) determines that the updated underlying model(s) within a pair of data models should not be fused, the system can send a signal that either adjusts an operation of the system (e.g., stops operation of the system or sub system, utilize only the best updated underlying model), or provides a notification to a user (e.g., presented on a display) that the updated models should not be fused (e.g., when previous iterations of these underlying models have fused in the past). This can allow real-time adjustment to optimize performance of a system, based on changing conditions of the underlying data models.

As another example, some non-limiting examples can provide improvements for alternative implementation logistics. These systems and methods can be used to choose and then integrate models optimizing different parameters of interest. For example, one can select for predicted gain, maximal possible gain, total expected gain, income, appreciation, resistance to loss, volatility, stability in a sell-off, gain in a sell off, and the like. One can also optimize on given parameters (e.g., a stock whose beta will increase over time). Similarly, it is apparent that any number of models having any desired set of these characteristics (either with the same or different outcome characteristics) can be combined to identify target financial instruments to purchase or to sell, now or in the future. In each case, the method remains the same, what is altered are the processes (e.g., data models) that are used to feed information into the method. It is similarly apparent that it is up to the user as to whether they choose to buy/sell the top 1, top 2, top 3, etc., number of choices. The user may choose to make this decision, or may choose to use the method to consider multiple scenarios. In one case, these systems and methods first optimize a series of models that focus on each output characteristic (e.g., predicted gains), then integrate each of these models (e.g., a two stage model using these systems and methods twice). The method allows the specific models in use to be dynamically determined based on the changing correlations).

As another example, some non-limiting examples can provide improvements for arbitrage (e.g., improve arbitrage opportunities). These systems and methods can be used to monitor combinations of financial instruments for arbitrage opportunities. Because of the optimal computational efficiency of the method, one can examine multiple combinations for potential arbitrage, for example, it is expected that one can, in real time, simultaneously monitor all US stocks in combinations with all major expected potential market shifts and responses. If more limited schemes are used, it is expected that the speed advantage given by the method will provide a competitive edge to the user. In one non-limiting example, the user can set thresholds for likely future arbitrage opportunities (e.g., using models that predict arbitrage opportunities, and thus setting action strikes when these systems and methods triggers a signal). In other words, use of these systems and methods to identify a probability rather than a specific outcome, and triggering a signal based on this.

As another example, some non-limiting examples can provide improvements for stop/loss and buy triggers (e.g., set trigger points, e.g., buy/sell, stop/loss and the like). These systems and methods can be used to monitor the potential price of a given stock under a series of market conditions. Because no true training set can exist (the market is never exactly identical), this problem cannot be solved using current approaches. These systems and methods, however, allow one to determine the best model or models under current, shifting conditions, and use this to assign probabilities to specific bounds being crossed, and thus triggering stop/loss orders or buy triggers. Under these conditions, it is now possible to decide at what percentage an individual wants to set a trigger, this increases flexibility, and provides a trading edge.

As another example, some non-limiting examples can provide improvements for automated trading systems. For example, these systems and methods can be used to create a substantially or fully (or partially) automated trading system. Given the computational efficiency, the systems and methods can theoretically always be able to either consider more options than conventional systems, or make decisions faster, or both.

As another example, some non-limiting examples can provide improvements for predicting factors related to investment fundamentals (e.g., weather, macroeconomic trends, and the like). These systems and methods can be used to improve the prediction of factors that affect fundamental asset pricing (e.g., weather/price interactions, supply/demand actions), which can be incorporated into a fully or partially automated trading system. Given the computational efficiency, such a system can theoretically always be able to either consider more options than a conventional system, or make decisions faster, or both.

As another example, some non-limiting examples can provide improvements for private equity. Private equity is an area of financial markets that is inherently low on data that can be used for training. The systems and methods described herein may prove useful in private equity by enabling the determination of the potential value of a start-up by identifying which models can be usefully combined in predicting success or failure and potential future value as well as, conversely, identifying non-informative models—that is, models whose signals fail to add to that provided by other models. The systems and methods also potentially enable one to judge the potential of two models to fuse under situations where little or no data exists or in which it would be impractical to obtain or to use. Model elimination, for example, can be triggered when it is determined that one model underperforms another by a sufficient enough that the correlation between such signals indicate that it can never (or not under circumstances deemed relevant) increase predictor accuracy/utility. Conversely, fusion is favored when it is determined that one model is sufficiently close in performance to another (and/or that their correlation is sufficiently low) that fusion of the two models is expected to increase the overall accuracy/utility of the current top model.

As another example, some non-limiting examples can provide improvements for initial investment assessments (e.g., by improving the prediction of factors that affect initial investment assessment). The different non-limiting examples of the systems and methods method described herein can be integrated for use in making decisions about initial investments. These systems and methods makes it possible to use model level fusions to optimize prediction about the future value of an acquisition, even in situations such as non-public companies, uncommon business areas, tech, IPOs, political or economic instability, and the like, where past experience is of little direct value and one cannot cross validate the models. Both the general case and pre-specified conditions can be studied. One can, for example, determine which set of models should be combined to optimize target outcome. Outcomes can be varied dependent on final desire, e.g., to balance a portfolio, to safely carry portion of investment to offset a different high risk investment, and the like. Models can be based on conditions (e.g., those that predict market share, market total value, time to market, regulatory approval and the like). Potential investments can be compared (e.g., via rank fusion) or absolute estimates can be obtained (e.g., via score fusions). The method enables improved modeling across the entire range of the predictions of one or more models (or one or more situations (e.g., the same model under changing market conditions) so as to detect and accurately capture weaker, rarer, or more complex signals by both amplifying signal and improving pre-acquisition understanding of the signal itself.

As another example, some non-limiting examples can provide improvements for the addition of complementary financial models (e.g., by adding additional models that can alter alter/improve the prediction of future valuations). Start-up and other private equity funded companies are inherently subject to relevant market conditions (e.g., an increase in the competitors from 0 to 1 is more serious than the increase in a grocery store's competitors from 100 to 101), and there is inherently little relevant data for them to work through potential joint modeling. It is clear that there is both a need for generally creating and introducing complementary models to improve performance via fusions, but also the ability to respond rapidly as to a need for models arising in a given time. The systems and methods optimally determine a potential model or models that would best integrate with existing model to improve the prediction of future prices, market conditions, and the like. The method enables this type of adaptation to occur either statically (e.g. the system adds a model and produces a different series of models), or adaptively (e.g., in the presence of signal A (e.g., a market decrease), adapts and uses configuration of model A′, but in the presence of signal B (e.g., high volume, market increasing), adapts and uses confirmation of models B′)—adding or inactivating models as needed. It is apparent that gains can be reflected in output, computational speed, energy utilization, sensitivity, specificity, and the like.

As another example, some non-limiting examples can provide improvements for managing a portfolio of companies (e.g., to improve management of a portfolio of early stage companies). The different non-limiting examples of the systems and methods method described herein can be integrated for use in assembling a portfolio. For example, one can assess the relative future value of sets of related assets, and use this information to decide which of two or more opportunities to pursue. One can choose higher and lower risk assets in the portfolio, or to balance risks by using the method to identify the leader in each category, one can determine whether assets are priced equivalently (e.g., have equivalent expected gains per cost of carrying the asset or relative to initial investment, which ties up capital). One can also use these relative rankings to decide which opportunity to sell off, or to continue to invest in.

As another example, some non-limiting examples can provide improvements for alternative implementation logistics (e.g., by choosing and integrating models, and optimizing different parameters of interest). For example, one can select for predicted gain, maximal possible gain, total expected gain, income, resistance to loss, and the like. One can also optimize on given parameters, (e.g., projected user base under different economic scenarios)—a usage that may well leverage the ability to do these kinds of predictions without training data. Similarly, it is apparent that any number of models having any desired set of these characteristics (either with the same or different outcome characteristics) can be combined to identify potential acquisition or funding targets to fund or to sell, now or in the future. In each case, these base systems and methods remain the same, what is altered are surrounding processes/methods (e.g., algorithms) that are used to feed or utilize the information. In one non-limiting example, these systems and methods first optimize a series of models that focus on each output characteristic, then integrates each of these models (e.g., a two stage model using these systems and methods twice).

As another example, some non-limiting examples can provide improvements for managing and adapting multiple model systems (e.g., by automating the management of multiple model arrays). For example, these systems and methods can determine whether the potential addition/activation of different models can add information or conversely, where deletion or removal of a model either reduces resource usage at minimal/acceptable cost or improves signal fidelity by reducing, for example, false negatives. As one non-limiting example, one could wish to use a different set of models based on given market indicator, for example, the relative performance of IPOs vs the general market; tech vs non-tech consumer goods, economic indicators. In a different domain with a related implementation, one can envision that the switch in models also changes target outcomes (e.g., by maximizing potential gains instead of minimizing potential losses). The method enables this type of adaption to occur either statically (e.g., the system adds/subtracts a model and produces a different series of models), or adaptively (e.g., in the presence of signal A, adapts and uses configuration of systems A′, but in the presence of signal B, adapts and uses confirmation of systems B′)—adding or inactivating models as needed. It is apparent that gains can be reflected in output, scanning speed, energy utilization, sensitivity, specificity, and the like. The systems and methods also can monitor outputs and use this information to manage the array. The systems and methods can also optimally identify potential elimination of models (e.g., altering the tradeoffs between false negatives and false positives that would best integrate with existing models to improve false positives or false negatives with regards to sensor outputs). Adaptation benefits can be reflected in the users choice of input (e.g., resistance to failure, probability of gain of >X %/year, and the like). It is apparent that this adaptation can also be deliberately triggered or activated by an influence external to the modeling system (e.g., an unexpected real-world event that leads to expert opinion can be created as a model).

As another example, some non-limiting examples can provide improvements for insurance (e.g., by enabling improved determination of the pricing of individual policies (e.g., improved risk determination) as well as by improving overall policy portfolios). The systems and methods can be used to determine which models can be usefully combined in predicting both relative and absolute risk for a given policy, as well as, conversely, by identifying non-informative models—that is, models whose signals fail to add to that provided by other models. The method also potentially enables one to judge the potential of two models to fuse under situations where little or no data exists or in which it would be impractical to obtain or to use. This occurs, for example, in the insurance of large, unique structures such as office buildings. Model elimination can be triggered when it is determined that one model underperforms another by a sufficient enough that the correlation between such signals indicate that it can never (or not under circumstances deemed relevant) increase accuracy/utility. Conversely, fusions are favored when it is determined that one model is sufficiently close in performance to another and that fusion of the two models is expected to increase the overall accuracy/utility of the current top model.

As another example, some non-limiting examples can provide improvements for value determination of a single policy (e.g., by making decisions about the risk of a given policy). The systems and methods makes it possible to use model level fusions to optimize prediction about the future risk of a given policy, even in situations such as unique structures, uncommon business areas, IPOs, political or economic instability, and the like where past experience is of little direct value and one cannot cross validate the models. Both the general case and pre-specified conditions can be studied. One can, for example, determine which set of models should be combined to optimize pricing. Outcomes can be varied dependent on final desire (e.g., to balance a portfolio, maximize return on a given policy, avoid policies that cross a specific risk threshold, and the like). Models can be based on conditions (e.g., political stability, weather conditions, local neighborhood quality, government policies, competing insurance companies, and the like). Potential policies can be compared (e.g., via rank fusion) or absolute estimates can be obtained (e.g., via score fusions). The method enables improved modeling across the entire range of the predictions of one or more models (or one or more situations, e.g, the same model under changing market conditions) so as to detect and accurately capture weaker, rarer, or more complex signals by both amplifying signal and improving pre-acquisition understanding of the signal itself.

As another example, some non-limiting examples can provide improvements for the addition of complementary insurance-risk models. For many insurance scenarios there is inherently little relevant data for them to work through potential joint modeling. It is clear that there is both a need for generally creating and introducing complementary models to improve performance via fusions, but also the ability to respond rapidly as to a need for models arising in a given time. The systems and methods can optimally determine a potential model or models that would best integrate with existing model to improve the risk prediction. The method enables this type of adaptation to occur either statically (e.g., the system adds a model and potentially produces a different series of output models), or adaptively (e.g., in the presence of signal A (e.g., political change, increased local crime rate), adapts and uses configuration of model A′, but in the presence of signal B (e.g., increased spending on police and fire), adapts and uses confirmation of models B′)—adding or inactivating models as needed. It is apparent that gains can be reflected in output, computational speed, energy utilization, sensitivity, specificity, and the like, although it is expected that potential risk is the dominant outcome. It is also apparent that this adaptation can also be deliberately triggered or activated by an influence external to the modeling system, (e.g., an unexpected real-world event that leads to expert opinion can be created as a model).

As another example, some non-limiting examples can provide improvements for temporary or permanent removal of financial models. These systems and methods can identify potential elimination of models (e.g., altering the tradeoffs between false negatives and false positives that would best integrate with existing models to improve false positives or false negatives with regards to fusion models). The goal being to remove from fusions those models that are non-informative or mis-informative. Model elimination can be triggered when it is determined that one model underperforms another by a sufficient enough that the correlation between such signals indicate that it can never (or not under circumstances deemed relevant) increase accuracy/utility. These systems and methods enable this type of adaption to occur either statically (e.g., the system resets and uses a smaller series of models) or adaptively (e.g., in the presence of signal A, adapts and uses configuration A′, but in the presence of signal B, adapts and uses confirmation B′). It is apparent that gains can be reflected in the users choice of input (e.g., all claim causes, fire, crime, and the like). It is also apparent that this adaptation can also be deliberately triggered or activated by an influence external to the modeling system (e.g., an unexpected real-world event that leads to expert opinion can be created as a model).

As another example, some non-limiting examples can provide improvements for managing a portfolio of companies (e.g., by assembling a portfolio of insurance policies). For example, one can use rank fusion to determine those policies that are relatively low performing or high performing by any of these metrics, and do so with more accuracy than existing approaches. One can choose higher risk/higher premium and lower risk/lower premium assets in the portfolio, or one could balance risks by using the method to identify the leader in each category. One can also determine whether assets are priced equivalently (e.g., have equivalent expected gains per unit risk). One can seek to maximize gross premiums collected, expected profit, potential profit, minimize maximal risk, minimize expected risk, and the like.

As another example, some non-limiting examples can provide improvements for alternative implementation logistics. For example, one can select for predicted gain, maximal possible gain, total expected gain, income, appreciation, resistance to loss, volatility, stability in a sell-off, gain in a sell off, and the like. One can also optimize on given parameters (e.g., a stock whose beta will increase over time). Similarly, it is apparent that any number of models having any desired set of these characteristics (either with the same or different outcome characteristics) can be combined to identify target financial instruments to purchase or to sell, now or in the future. It is similarly apparent that it is up to the user as to whether they choose to buy the top 1, top 2, top 3, and the like, number of choices, and whether they use one or model fusion models to make their choices.

As another example, some non-limiting examples can provide improvements for model array adaptation. These systems and methods can optimally identify potential adaptions to the interpretation of model signals (e.g., altering the tradeoffs between false negatives and false positives that would best integrate with existing models to improve target acquisition). The systems and methods enable this type of adaption to occur either statically (e.g., the system resets and produces a different series of models) or adaptively, (e.g., in the presence of signal A, adapts and uses configuration A′, but in the presence of signal B, adapts and uses confirmation B′). It is apparent that gains can be reflected in output, scanning speed, energy utilization, sensitivity, specificity, and the like. One can also focus on target outcomes (e.g., maximizing potential gains, minimizing potential losses, maximizing potential gains at a given risk level or for given market predictions, and the like).

As another example consider a series of systems (e.g., informatics outputs): A₁, A₂, A₃, A₄, A₅ . . . A_{N}. These (A₁, A₂, A₃, A₄, A₅ . . . A_{N}) can be passed through any of the systems and methods described herein to identify an optimal fusion (e.g., an optimal pair). Then, the identified optimal pair can be fused to increase accuracy: A₁, A₂, A₃, A₄, A₅ . . . A_{N}→(systems and methods herein)→Aₚ, A_{q} where p,qε(1−N)→A_{pq} (where A_{pq} is the fused pair of data models). Then the user can use this fused pair as their model, for example, buying the top stocks, selling the bottom stocks, or standard combinations such as spreads, straddles, and the like.

As another example consider two companies, A, and B that wish to work together, without directly sharing information. As an exemplar, consider two financial companies that have evaluated a series of potential purchases. For example, consider a series of systems, e.g., informatics outputs in company A: A₁, A₂, A₃, A₄, A₅ . . . A_{N}. These can then be passed through these systems and methods to identify an optimal fusion (e.g., A₁, A₂, A₃, A₄, A₅ . . . A_{N}→(systems and methods herein)→Aₚ, A_{q} where p,qε(1−N)). Then these models can be fused to increase accuracy (e.g., A₁, A₂, A₃, A₄, A₅ . . . A_{N}→(systems and methods herein)→Aₚ, A_{q}→A_{pq}; where A_{pq} is the fused data model that was previously determined as optimal). Similarly, consider a series of systems, e.g., informatics outputs in company B: B₁, B₂, B₃, B₄, B₅ . . . B_{M}. These can then be passed through these systems and methods to identify an optimal fusion (e.g., B₁, B₂, B₃, B₄, B₅ . . . B_{M}→(systems and methods herein)→Bᵣ, Bₛ where r,sε(1−M). Then these models can be fused to increase accuracy (e.g., B₁, B₂, B₃, B₄, B₅ . . . B_{M}→(systems and methods herein)→Bᵣ, Bₛ→Bᵣₛ; where Bᵣₛ is the fused data model that was previously determined as optimal). Then the systems and methods (e.g., a computing system, computing device, and the like) can identify the best combination of the four underlying data models (e.g., A_{pq}, Bᵣₛ→(systems and methods herein)→A_{pq}, Bᵣₛ A_{pq}, or Bᵣₛ→and choose the best of these four models. A user, for example, can use the best model at any given moment as their model, for example, buying the top stocks, selling the bottom stocks, or standard combinations such as spreads, straddles, and the like. In some cases, by running this as a black box algorithm, with hidden inputs and outputs, it is possible for the companies to work together freely without reveling any trade secrets (e.g., a computing device such as an external server, receiving data, or models, or both and implementing the methods herein).

In one specific non-limiting example envisioned, it is apparent that it will be possible to pre-set certain accuracy/correlation combinations to use either a look up table, or correlation alone, or to set criteria (fixed or evaluated live) to choose between leveraging a look-up table or mathematical calculations.

As another example, some non-limiting examples can improve the performance of auctions for internet add placement (e.g., banner ads on a website). The value of a given slot (e.g., location such as the website, and the specific location on the webpage) can be determined by what someone will pay, inherently based on what models they build says the slot is worth. The systems and methods here can be used to speed up this determination (e.g., by better combing these estimation models), add additional models etc. Both situations would be advantageous to both the buyer of the slot and the seller of the slot.

As another example, the systems and methods herein can provide a user (e.g., a pharmaceutical company) with concrete rationales to provide leveraged arguments to an agency (e.g., the FDA, an Institutional review board, etc.) that only one or a limited subset of a plurality of tests (e.g., data models, such as for a study) need to be completed to appropriately determine the efficacy (or safety) of a product (e.g., a drug, procedure, etc.), at least because a multiplicity will not improve the information recovered. This can save money and time for both parties, provide less impact on patient, possibly use tests that can be done quicker (e.g., point-of-care instead of in hospital, etc.), and can be evaluated relatively quickly (e.g., without ever testing the combination in a full series).

As another example, the systems and methods herein can provide a user (e.g., a pharmaceutical company) with concrete rationales to provide leveraged arguments to an agency (e.g., the FDA, an Institutional review board, etc.), that a given test should be done (e.g., on a drug, markers in a study, diagnostics) because a multiplicity will improve information recovered (e.g., information regarding the efficacy, safety, etc.). This can save money and time for both parties (e.g., by preventing a need to rerun a study), provide less impact on patients (e.g., by improviong outcomes, reducing time in trial, reducing number of patients, different choice of tests), possibly use tests that can be done quicker (e.g., point-of-care instead of in hospital, etc.), and can be evaluated relatively quickly (e.g., without ever testing all possible combinations in a full series). This can be especially true when the user can appropriately show that a cheaper (and/or quicker test or combination of tests) can be substituted with either the same or a greater amount of information recovered such as complementary information (e.g., the efficacy, safety, etc. of a product, such as a pharmaceutical drug). This can then allow selection for reasons other than medical information (e.g., cost, accessibility, stability, use in remote regions, etc.) since medical information itself can be held constant (or potentially improved).

As another example, the systems and methods herein can provide improvements for search and information retrieval (e.g., deep searching). Standard information retrieval leverages information present at the ends of distributions (e.g., most key hits are on Google's first page), but it is harder and the algorithms are of less use for less strong hits. Because these systems and methods herein can leverage and use information across the entire distribution, it can enable the discovering of relevant documents at lower priorities. The approach is robust, deep, and enables identifying maximally diverse hits. In specific cases, these systems and methods can be used for targeted area search and retrieval. For example, via a series of key words, an overall type of search can be used to activate a series of models and deactivate others, thus enabling a targeted search. These systems and methods enable this type of selected search to be conducted dynamically.

As another example, the systems and methods herein can provide improvements by, based on, for example, for or against fusion of a pair of data models, add models (e.g., to a pool of data models, remove models (e.g., from a pool of data models), group models by different diversity thresholds, allow for adaptive and changing evaluation of data models. The application of these systems and methods herein are far reaching being applicable to chemistry (e.g., biochemistry, such as antibodies, DNA analysis, other genetic analyses), drug development (e.g., pharmaceuticals, biopharmaceuticals), sensors systems (e.g., multi-sensor systems) such as avoidance or evasive systems (e.g., obstacle avoidance systems) such as unmanned vehicles (e.g., self-driving cars, semi-autonomous vehicles), combat systems (e.g., tanks, fighter jets, etc.), other automation systems for vehicles (e.g., flight corrections for airplanes, helicopters, and other flying vehicles), complex control processes for factories, manufacturing plants (e.g., chemical synthesis plants, chemical extraction plants), treatment plants (e.g., water treatment plant, waste treating plant), utilities (e.g., power grid management, such as to respond to fluctuations in power demand), emergency systems (e.g., faster and better emergency responses), sports (e.g., better strategies, injury prevention, etc.), and sleep (e.g., determining specific sleep stages, and determining different sleep disorders).

As another example, the systems and methods herein can provide improvements for the feedback management and adaption of upstream systems. For example, systems and methods herein can optimize (or improve) the upstream series by, for example, removing systems (e.g., or data models) that contribute redundant information, removing systems (e.g., or data models) that contribute low quality information, removing sets of systems (e.g., or data models) whose information is already captured in another system (e.g., or data models) (e.g., if C is the product of the fusion of A and B, remove A and B), remove systems (e.g., or data models) whose information does not have a sufficient benefit to cost ratio, identify a potential system (e.g., or data models) or set of systems (or their characteristics) that can be used to improve current models, and identify a series of systems (e.g., or data models) that attain maximal (or functionally acceptable) accuracy at lowest cost (e.g., by sequential elimination of systems). In some cases, the systems and methods can perform a systematic search, for example, these systems and methods determine whether the potential addition/activation of different models can add information or conversely, where deletion or removal of a model either reduces resource usage at minimal/acceptable cost, or improves signal fidelity by reducing, for example, false negatives. These systems and methods can optimally identify potential elimination of models, e.g., altering the tradeoffs between false negatives and false positives that would best integrate with existing models to improve false positives or false negatives with regards to model outputs. In some cases, these systems and methods can monitor the output and use this information to manage the array. It is apparent that this adaptation can also be deliberately triggered or activated by an influence external to the modeling system, e.g., an unexpected real-world event that leads to expert opinion can be created as a model. In one non-limiting example, data models are deactivated when these systems and methods determine that one data model underperforms another by a sufficient enough that the correlation between such signals indicate that it can never (or not under circumstances deemed relevant) increases overall model accuracy/utility. It is apparent that this adaptation can also be deliberately triggered or activated by an influence external to the modeling system, e.g., an unexpected real-world event that leads to expert opinion can be created as a model.

As another example, the systems and methods herein can provide improvements for combined multi-stage implementation(s) (e.g., by optimizing sequential fusions). As one non-limiting example, the upstream series of systems can be chosen using domain expertise, e.g., consider N models that predict the highest potential return between ten stocks, M models that predict the lowest risk of loss between the same ten stocks, and a third set of O models that predicts the highest expected return between ten stocks. These systems and methods can operate on each of the three domains independently, then decide whether the overall series should be fused. In another non-limiting example, the upstream series of systems can be chosen using math, e.g., consider N models that predict highest potential return between ten stocks, M models that predict lowest risk of loss between the same ten stocks, and a third set of O models that predicts highest expected return between ten stocks. The total series, N+M+O systems can be clustered (e.g., by hierarchical cluster analysis) and each possible binary fusion can be assessed, and only those beneficial fused, beginning from the leaves and moving to the branches. In another envisioned non-limiting example, it is possible to leverage the predefined decision tree that these systems and methods follow to determine whether it is possible to arrive at later steps in a set of sequential fusions with better fusion partners (e.g., less correlated models) using one set of fusions vs another, or using one set of models vs another. This can be scripted and embedded within a system to, for example, for any set of models, determine an optimal or near optimal path of sequential fusions, either by algorithmic approaches (e.g., a clustering-like approach with an appropriately weighted distance algorithm) or by simulation of all possible series of fusions.

### EXAMPLES

The following examples have been presented in order to further illustrate aspects of the disclosure, and are not meant to limit the scope of the disclosure in any way. The examples below are intended to be examples of the present disclosure and these (and other aspects of the disclosure) are not to be bounded by theory.

The results below are compelling and reveal a precise, quantitative relationship between Diversity and Accuracy, which are referred to as the diversity of ranks and accuracy (e.g., the “DIRAC” framework). The results held for both simulated and real-world biological data. Secondary analysis demonstrated that the relationship observed is at least partially dependent on the rankings of the samples in the classification systems, and not a direct result of their scores. We also discuss the potential implications, applications and extensions of this framework.

A breakthrough mathematical framework that accurately forecasts the utility of combining predictive or descriptive mathematical models without requiring further cross-training or cross-validation, and intrinsically resolves most or all weighting issues has been developed and validated. This framework, which is embedded in various forms in these systems and methods, is a new, essentially complete understanding of how and when multiple data models (or algorithms) can work together, which, in turn, enables the user to optimally use nearly any existing general or domain-specific data analysis methods without requiring additional training sets, or even having the models developed on overlapping data sets.

Some non-limiting examples of the disclosure, by determining how the different components and applicable characteristics of mathematical models interact, address and encompass almost all MSC-fusion problems. For example, for most fusions, non-limiting examples of the disclosure can determine whether or not a fusion will be beneficial with 100% accuracy. For a very small percentage of fusions, the method can determine that the fusion will have minimal effect on model accuracy, but the absolute direction is subject to stochastic/sampling variation and cannot be predetermined. Under most conditions tested to date, our method also offers quantitative predictions that are accurate to within 1%.

The combination of the broad potential of information fusion and the specificity of the results obtained suggests this work has potential applications in far-ranging areas including insurance pricing, risk management, clinical biomarker development, personalized medicine, clinical trial enrollment, portfolio management, information retrieval, and sensor optimization, among others. A subset of these are enumerated in the claims. Furthermore, this framework points the way to additional insights that can further optimize predictive or descriptive mathematical MSC-fusions, tailor these developments for specific domains and applications, improve the performance of individual underlying algorithms and design novel algorithms with properties pre-optimized for subsequent fusion-level enhancements.

An averaging of model outputs was exhaustively explored to determine whether there are conditions in which an average of the models will consistently outperform the better of the two individual models, which is pairwise system fusion in its most basic instantiation. In this case, the term “system” represented anything that gives a single numerical assignment to every sample in a population of samples, although “systems” generally can be as complex as fitted ensemble classifiers, or as simple as single measurements (e.g., such as fasting blood glucose level). Thus, to avoid confusion, we referred to all of these diverse models and measurements from this point forward as “scoring systems,” and denoted them as “SS_{A}”, “SS_{B}”, and the like, having scale the scoring systems' outputs from 0-1.

To determine the relationship between the characteristics of the input systems and outcome of their fusion without being confounded by domain-specific factors, analytical noise, and/or classification errors (such as incorrect labeling), simulated scoring system data was initially generated using probability distributions designed to approximate those of the data that are typically seen in real-world applications. Most frequently, this is a mixture of two Gaussian family distributions; these distributions, hereafter class 1 [denoted C₁] and class 2 [denoted C₂], may represent, for example, disease and control populations. Thus, each fusion event would have C₁_SS_{A} (e.g., denoting scoring system A for class 1), as well as C₁_SS_{B}, C₂_SS_{A}, and C₂_SS_{B}. A standard Gaussian distribution can be combined with an exponential distribution to create an exponentially modified Gaussian distribution (“EMG”), which can have a significantly wider tail, more accurately representing the distributions of many types of real-world biological (and many non-biological) measurements.

Data samples drawn from the C1 and C2 distributions from one scoring system (e.g., C₁_SS_{A}, C2_SS_{A}) can be interpreted as scores for the purpose of classification, with the score of a single data sample within SSA reflecting the likelihood of that sample having originated from C1 or C2. Altering the relative difference between the means of C1 and C2 will affect this likelihood, as will altering the standard deviations. When the C1 and C2 distributions are fully separated, it represents a perfect classifier (e.g., all the C1 scores will be less than the C2 scores, so knowing the score conveys complete information about which distribution [C1 or C2] the sample was drawn from), and conversely, when there is nearly complete overlap, the classifier will be closer to a random guess, and knowing the score will not convey as much information about the distribution of origin of the sample.

A scoring system having greater separation between the C1 and C2 probability distributions will have higher performance, e.g., AUROC. The AUROC is a generally accepted and useful metric of classifier performance, as it captures the tradeoff between sensitivity and specificity without requiring the selection of a specific threshold, which is required for other performance metrics such as misclassification rate. AUROC is thus a single number that captures overall performance, and, due to this utility, it was the metric on which was focused.

To explore the effect of the C1/C2 distribution parameters on pairwise fusion performance, a large pool of scoring systems were created, with the means, standard deviations, and exponential decay parameters sampled randomly from wide uniform distributions. As noted above, the random variations in the difference between the means of C1 and C2, and the relative standard deviations of the distributions lead to a broad distribution of AUROCs (e.g., accuracy). Randomly selected pairs of scoring systems (SSA, SSB) were then fused by averaging individual points (equivalent to the scores from a synthetic sample) from C1_SSA with those from C1_SSB, and from C2_SSA with those from C2_SSB, and calculating the mean score of each pair. This approach simulates a relatively uncorrelated pair of scoring systems both evaluated on the same set of observations (e.g., a blood glucose [e.g., SSA] and a body weight [e.g., SSB], both predictors measured on all members of a set of individuals). The AUROCs of SSA and SSB are referred to as AUROCA and AUROCB, respectively. AUROCM is referred to as the AUROC of the superior input classifier (i.e., max[AUROCA, AUROCB]). The AUROC of the fused system (hereafter, AUROCSF[AB] for the score fusion of SSA and SSB) was measured, and compared with AUROCM (e.g., specifically where ΔAUROCSF[AB]=AUROCSF[AB]-AUROCM). This process was repeated across the large pool of scoring systems, which allowed us to explore how the C1/C2 distribution parameters influence the AUROCSF[AB] of the resulting scoring systems and the ΔAUROCSF[AB].

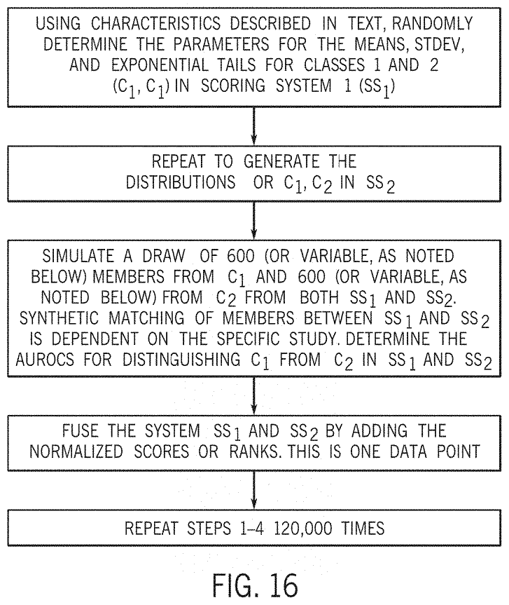

FIG. 16 shows a graphic illustrating the process used to generate simulated data that includes a plurality of pairs of fused data models. To simulate C1 and C2 data using a Gaussian density function four parameters must first be chosen: the mean and standard deviation of the C1 distribution, and the mean and standard deviation of the C1 and C2 distribution. In order to enforce some degree of separation between the component distributions the mean of the C1 component was sampled at random from the range −5 to 0, and the mean of the C2 component was sampled at random from the range 0 to 5. The standard deviations of both components were then sampled uniformly and at random from a predetermined range (e.g., 1-20). With the sampling distributions parameterized, an equal number of samples were then drawn from the C1 and C2 distributions, and pooled. This created a single instance of a simulated C1 vs C2 scoring system. To explore the effect of the number of samples drawn (simulating the N of the experiment), this process was repeated many times, with the sample number ranging from 10-600/class. All systems scaled from 0 to 1. Although FIG. 16 shows the process repeating 120,000 times, in other configurations, such as with respect to the process 200 of FIG. 14, the process can be repeated any other suitable amount of times (e.g., 100,000, 12,000,000, with higher numbers of iterations having increased resolution and requiring increased generation time, and with lower numbers of iterations having a decreased resolution and requiring less generation time).

The procedure for generating synthetic exponentially modified data is much the same as described above, except that both a Gaussian and a separate exponential distribution must be parameterized separately for both the C1 and C2 data sets. The Gaussian distributions were randomly parameterized as described above, and the exponential distributions were parameterized by selecting the exponential mean uniformly and at random from a predetermined range. To sample exponentially modified data, a sample is first drawn from the Gaussian distribution, then a sample is drawn from the corresponding exponential distribution, and these samples are added together. This modified procedure was iterated as above to generate sets ranging in size from 10 to 600 per class. All systems scaled from 0 to 1.

Simulated scoring systems do not correspond to multiple measurements of a single set of phenomena. Thus, the scores that are sampled from the C1 and C2 distributions of each of the pair of systems can be explicitly paired in order to influence the apparent correlation of the two scoring systems. When the C1_SSA to C1_SSB and C2_SSA to C2_SSB pairings are done at random, the correlation between the two systems will be minimal. When the pairing is not random and (for example) low-scoring, mid-scoring, and high-scoring case (and respectively control) samples from SSA are paired with equivalent low-scoring, mid-scoring, and high-scoring case (respectively control) samples from SSB, a non-zero level of correlation can be induced. In the extreme case, with the points matched exactly by rank, the correlation will achieve a maximum value. Though the actual maximum value will also be influenced by other properties of the sampling distributions, if the distribution shapes are very similar, the maximum correlation will approach 1.0.

The process of fusion creates a single, new scoring system from two input scoring systems. In this study we examined the simplest manifestation of fusion, which simply averages the value of the two input systems in a pairwise fashion, after each input system has been scaled to fall between a minimum value of 0.0 and a maximum value of 1.0.

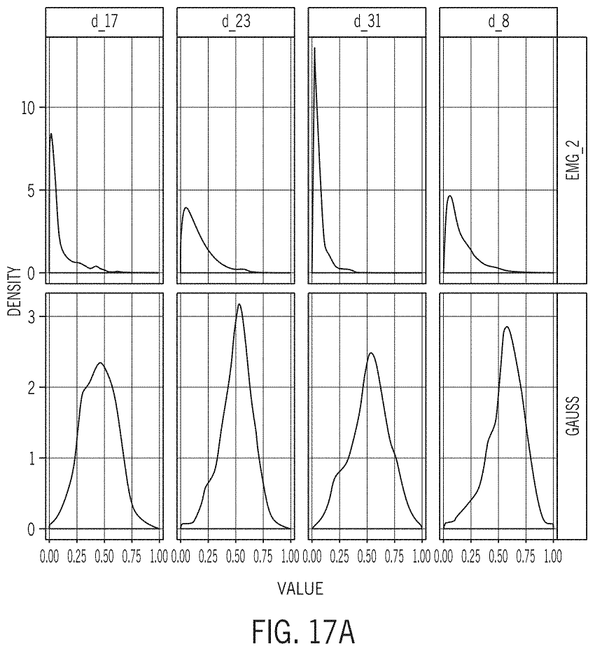

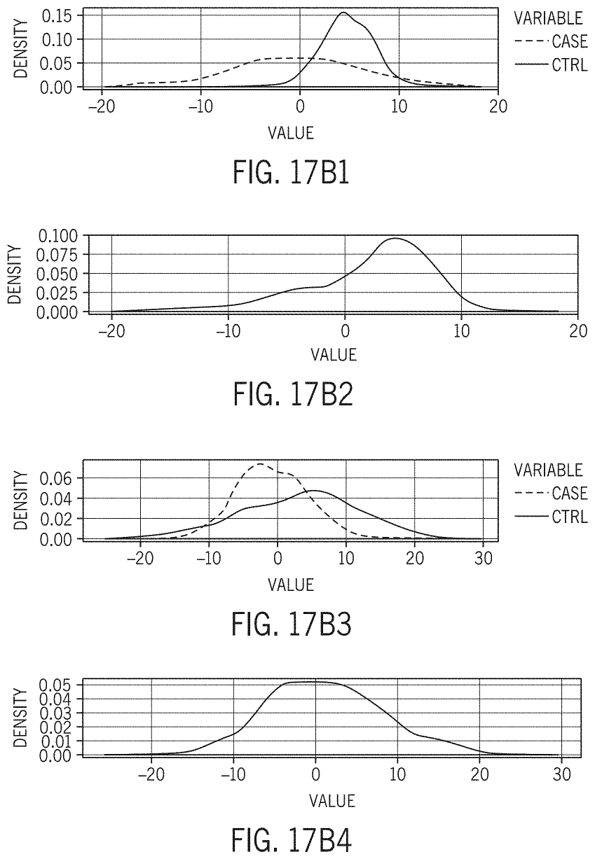

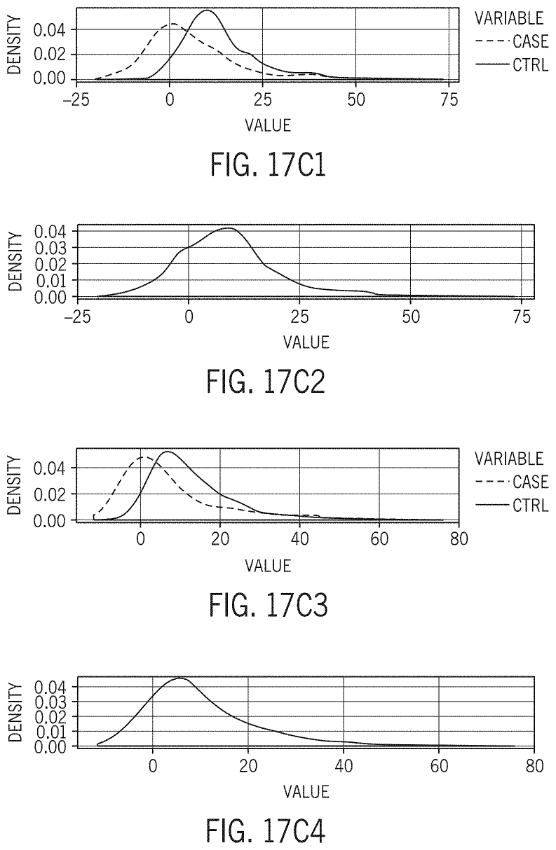

FIGS. 17A-17C4 shows various graphs illustrating how the simulated data was generated. In particular, panel A depicts the generation of simulated data where the bottom of panel A is the Gaussian distribution and the top is the Exponentially Modified Gaussian (EMG) distribution, with uniformly sampled values of standard deviation, and exponential decay (in the case of the EMG). The distributions shown were mean-scaled for easier visual comparison, but, for the main study, the mean was also uniformly sampled. Panel B of FIG. 17 shows examples of two sets of Gaussian scoring systems, with the mean and standard deviation values sampled from appropriate uniform distributions. Panels B.1 and B.2 of FIG. 17 together show 1 scoring system (e.g., SS1 in the report's nomenclature). Panel B.1 shows the two class distributions, e.g., C1 shown in red (e.g., the more broad distribution), and a second distribution, e.g., C2 shown in blue (e.g., the more narrow distribution). Panel B.2 shows the entire scoring system distribution. Panels B.3 and B.4 of FIG. 17 show equivalents for a second system, (e.g., SS2 in the report's nomenclature). A single fusion would involve fusing the score from the cognate observations in SS1 and those in SS2. Panel C of FIG. 17 are the same as Panel B of FIG. 17, except with the EMGs.

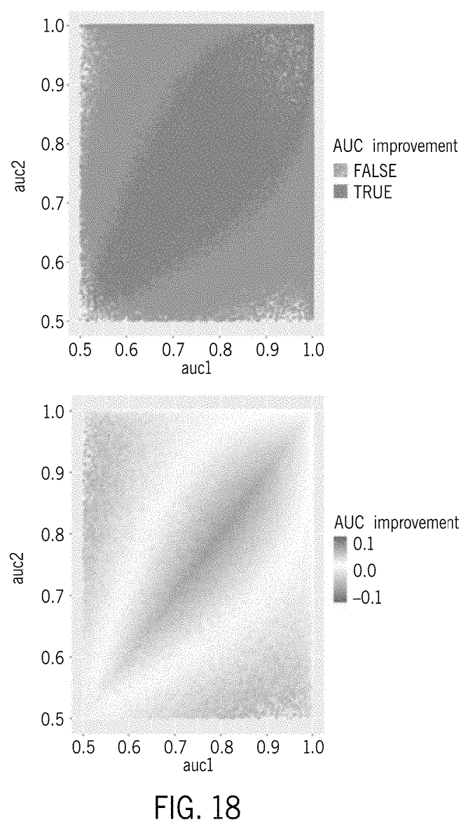

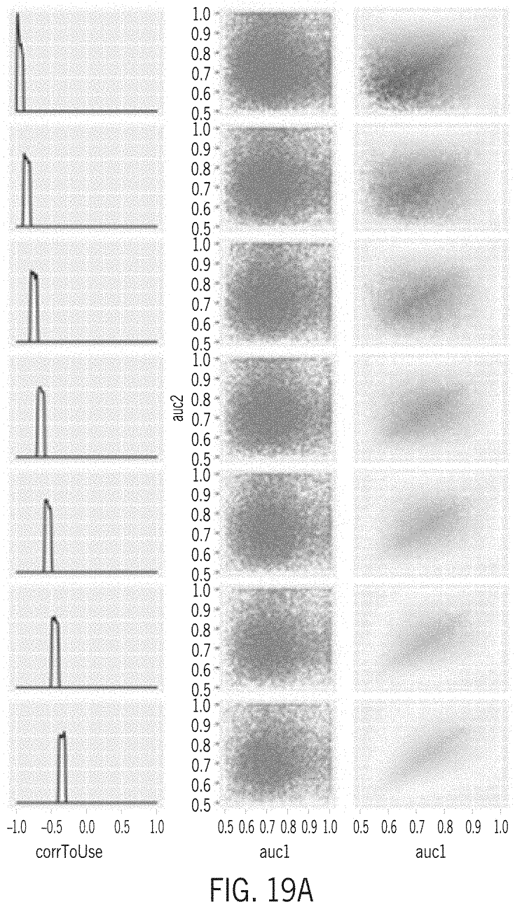

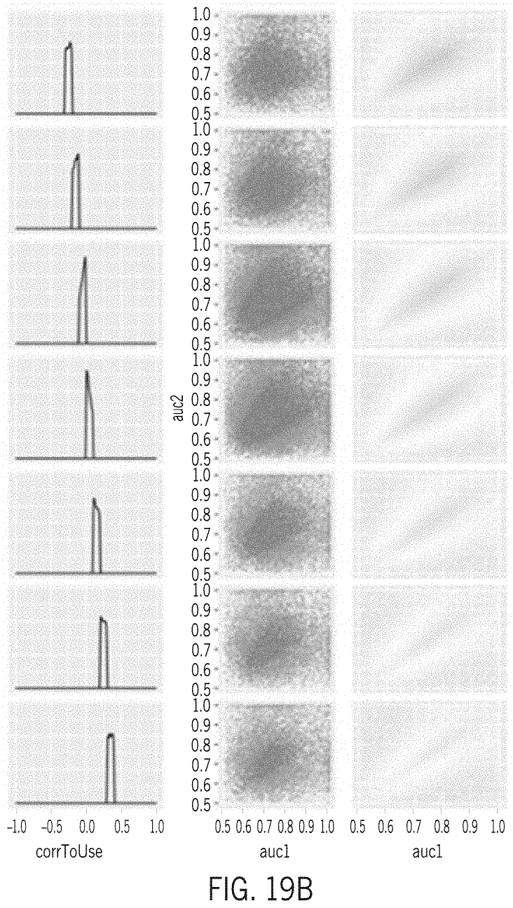

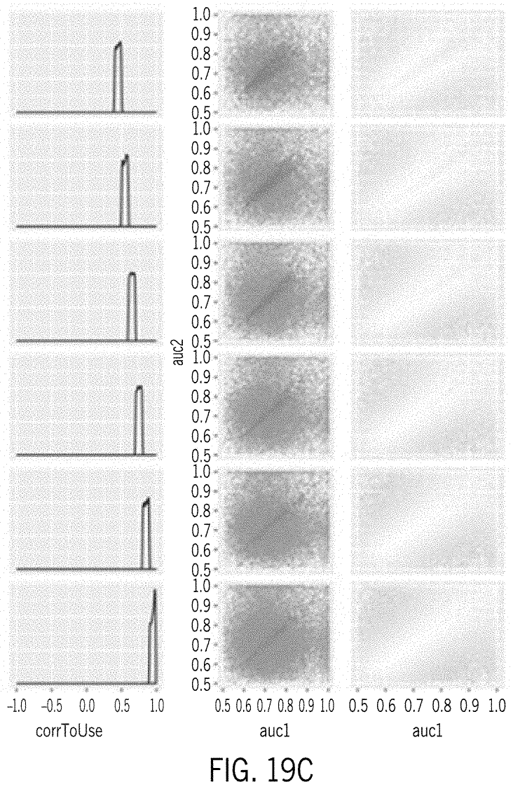

A visually striking result emerged when the improvement of the fused classifier systems (e.g., ΔAUROCSF[AB]>0; presented as binary true/false) was plotted as a function of the two input AUROCs (AUROCA and AUROCB; synthetic Gaussian data fusions and synthetic EMG data fusions. The space is separated into two distinct regions—one central region where ΔAUROCSF[AB]>0 (which is notably wider in Gaussian Fusions) and a peripheral region where ΔAUROCSF[AB]≤0. The separation of these two regions is not perfect. For example, between these two regions exists a relatively narrow band where the improvement (considered this way, as a binary outcome) is uncertain (see, e.g., FIG. 18). This area of uncertainty is notably wider in the EMG-derived fusions. Plotting ΔAUROCSF[AB] directly shows that the area of greatest AUROC improvement lies on a diagonal, symmetric about AUROCA=AUROCB (e.g., where the input scoring system performances are equal), and that a relatively wide area of zero-to-vanishingly-small improvement separates this area from that where the AUROCSF[AB] is not a net improvement over AUROCM (see, e.g., FIGS. 18 and 19A-19C).

A generally accepted principle in information fusion is that when combining two systems, the resulting performance is typically better when the two input scoring systems are both relatively accurate and diverse. Having identified an unexpected relationship between the effects of the relative performance “Accuracy” above in terms of the AUROCs, we now explored the role of “Diversity” using the average of the Pearson correlations between C1 in SSA and SSB, and C2 in SSA and SSB. This provided a simple measure of “Diversity” between SSA and SSB (hereafter, ΔPC(SSA,SSB)=diversity of scores as defined by Pearson Correlation). This allowed for the exploration and characterization of how different levels of diversity affect the results of pairwise scoring system fusions. For the purposes of this study, the average of the correlations within each class was used because it avoids complications due to the confounding dependence of the global correlation (e.g., correlation of the ranks independent of class) on the performance of the original scoring systems. Using substantially the same sampling algorithm described above (that produced uncorrelated scoring systems, see, e.g., FIG. 16), the within-class (C1 or C2) pairing of the samples were altered to produce different levels of correlation. Under the conditions used above, the mean correlation is near zero. When SSA and SSB samples are “encouraged” to come from similar “ends” of their respective distributions, the correlation between SSA and SSB increases. Conversely, when the SSA and SSB samples are encouraged to come from opposite “ends” of their respective distributions, the correlations become negative. A principal advantage of using simulated scoring systems like this is that, due to sample pairing, the correlation between two systems can be varied without changing the mean differences, the standard deviations, or any other parameter, which allows for an unbiased examination of the effects of correlation alone.

Higher diversity is associated with increased probability that fusion will improve performance (e.g., ΔAUROCSF[AB]>0) and with increases in the maximal improvement resulting from fusion. As the mean SSA-SSB correlation increased (see, FIGS. 18 and 19A-19C), the area where ΔAUROCSF[AB]>0 became more narrow, until at highly positive mean correlation values only a sliver of the originally wide area of improvement remained (see, FIGS. 18 and 19A-19C). The absolute gains in ΔAUROCSF[AB] at these high correlation values was also reduced, and the absolute gain in ΔAUROCSF[AB] was maximized in fusions between strongly negatively correlated scoring systems. (see, FIGS. 18 and 19A-19C). These results demonstrate that the performance of the fused system may not be directly dependent on the performance of each individual system, and it also reveals that the effects of correlation on the performance of any given fusion are relatively independent of the AUROCs of the two fused scoring systems. This supports the intuitively satisfying notion that, for the fusion of two scoring systems to outperform the best individual system, they must each contribute at least some different information about the samples being examined.

FIG. 18 shows graphs of plotted pairs of data models indicating which regions are associated with improved and decreased (or no) performance after fusion. In particular, FIG. 18 shows that the success or failure of Gaussian score fusions is predictable. Each panel of FIG. 18 was created using random sampling of pairwise Gaussian score fusions (of N=600/class). The x and y-axis of the plot indicate the performance (in AUROC) of SSA (e.g., “auc1” on FIG. 18) and SSB (e.g., “auc2” on FIG. 18) respectively. The color indicates the net improvement of the fusion AUROC (AUROCSF[AB]), compared to the better of the two input systems—AUROCM. In the upper panel of FIG. 18, blue is positive (ΔAUROCSF[AB]>0) (e.g., the central region) and red (e.g., the outer region) is no improvement (ΔAUROCSF[AB]≤0). The lower panel of FIG. 18 shows absolute ΔAUROCSF[AB].

FIGS. 19A-19C shows a series of graphics, for particular correlation intervals, illustrating whether or not pairs of fused data models increased the accuracy of the underlying data models within each pair. In particular, FIGS. 19A-19C shows various graphs (e.g., sub panels), for each correlation interval. For example the left most column, C, shows a graph of the restricted correlation interval for the Pearson correlation between the fused pair to a 0.1 unit range of window, from −1 to 1). Columns D and E (right of column C, where D is adjacent column C) show distributions of the random sampling of pairwise score fusions (N=600/class), similar to FIG. 18, but stratified by correlation.

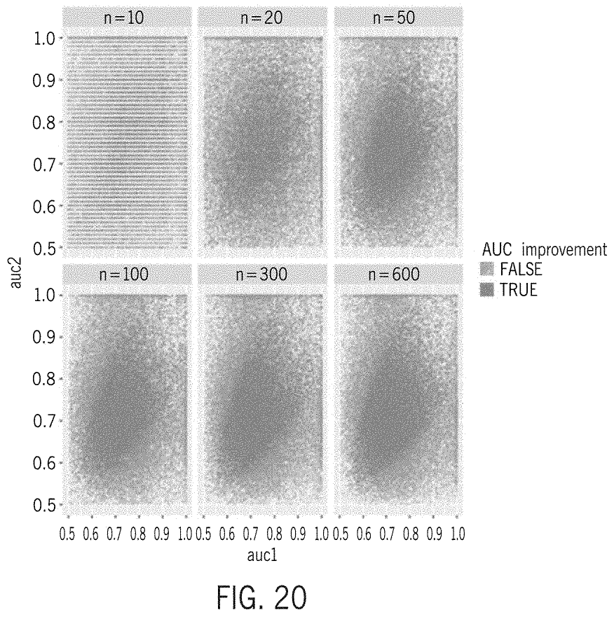

The number of samples (e.g., the N of the simulation, equivalent to the number of observations in a real-world dataset) also affects the accuracy of the fusion prediction, and directly underlies the interval of uncertainty between positive and negative ΔAUROCSF[AB]. For example, for relatively small sample sizes, the AUROC curve proceeds upwards and rightwards from the bottom left corner in a series of large jumps, and a single C1/C2 (case/control) reversal causes a correspondingly large change in the AUROC. Thinking of these smaller-N scoring systems as subsamples of a single larger-N scoring system, the AUROCs of the smaller systems represent a set of estimates of the larger-N system's AUROC, with the range of estimates derived from different subsamplings growing smaller as their N increases, eventually converging on the true population-level AUROC. The effect of N on prediction of fusion performance was systematically evaluated in a series in which each scoring system had 20, 40, 100, 200, 600, or 1200 total samples split evenly between C1 and C2 (see FIG. 20). These analyses reveal that increasing N shrinks the interval of uncertainty surrounding the transition zone between positive and negative ΔAUROCSF[AB]—revealing N as a critical/limiting component in the accuracy/robustness of forecasting ΔAUROCSF[AB]. Thus, what is shown is that the success of system-fusions are associated with three sets of parameters: (i) DPC(SSA,SSB); (ii) AUROCA and AUROCB, and (iii) the N observations used to create SSA and SSB.

FIG. 20 shows a series of graphics illustrating the impact of the number of samples (N) used to generate data models for pairs of fused data models, where the pairs of fused data models can predict whether or not to fuse a pair of data models. For example, as shown increasing the number of observations improves prediction precision. The effect of the sample size (N) on the sharpness of the boundary of the region of fusion improvement. The N shown represents the number of samples in C1 and C2 (simulated class numbers are balanced). Plotting artifacts are visible in the top left sub-panel, where the resolution of the total possible number of AUC values (with a total balanced N=20) is smaller than the points representing each fusion in the figure.

Perhaps of greatest importance, the results above suggested to that the performance of fusion is fundamentally rank-driven. Three factors led to expanding the analyses in the direction of ranks: (a) rank fusions have had success in prior combinatorial fusion studies, including several studies focusing on the conditions in which rank-fusions may outperform score fusions. and; (b) the AUROC itself is a rank-based metric, suggesting that the structure seen—the ellipsoid region of fusion improvement, is inherently driven by the geometric structure present at the level of ranks, not scores. Whenever the size of a dataset is fixed, it is possible to transform a scoring system (“SS”) into a ranking system (“RS”) by simply sorting the samples by their score, with the rank of a sample now equivalent to its place in the sorted ordering. Another factor is c), rankings are of particular utility in commonly encountered real world situations such as ranking candidates, e.g., by risk, for clinical trial enrollment, and rank-based statistical tests such as the Mann-Whitney U (“MWU”) test, which is equivalent to the AUROC, also directly indicates that successful fusions inherently improve statistical significance and power, as they are commonly employed to increase robustness in situations where violations of distributional assumptions are known to contribute excessive noise to the signal of interest.

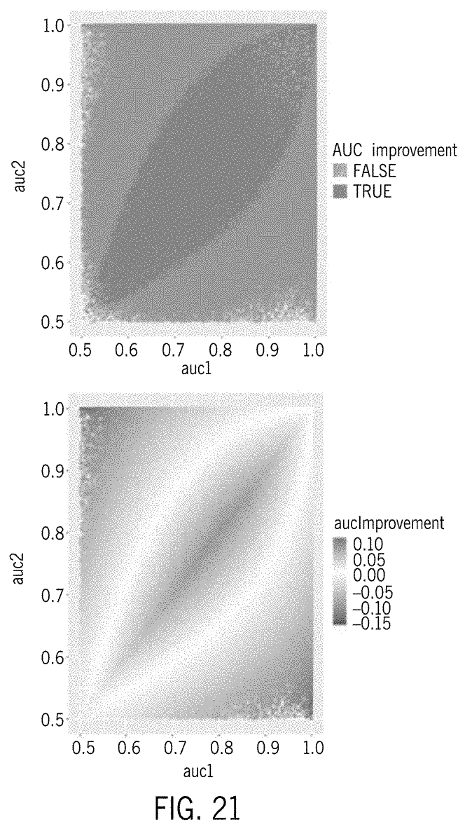

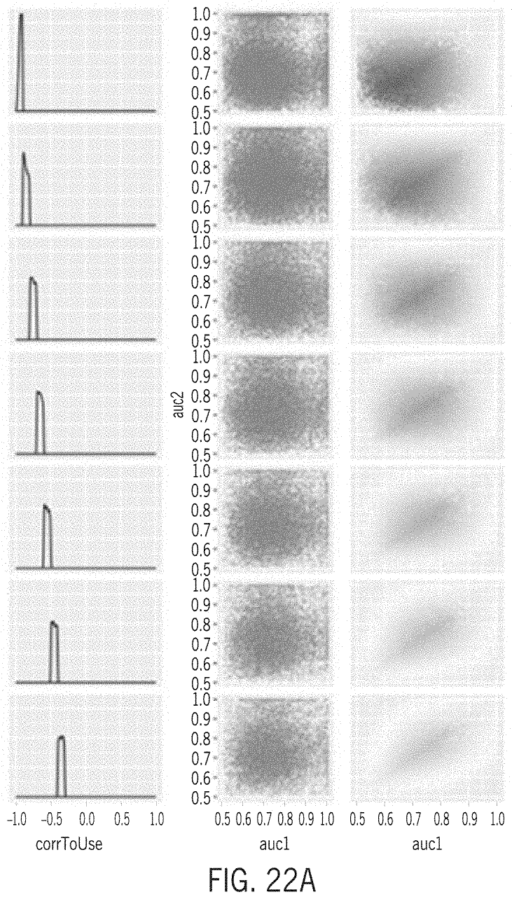

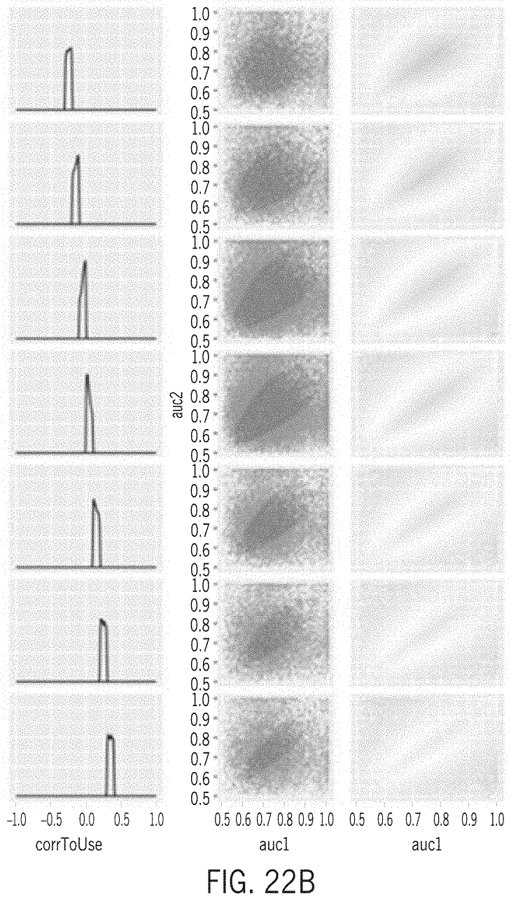

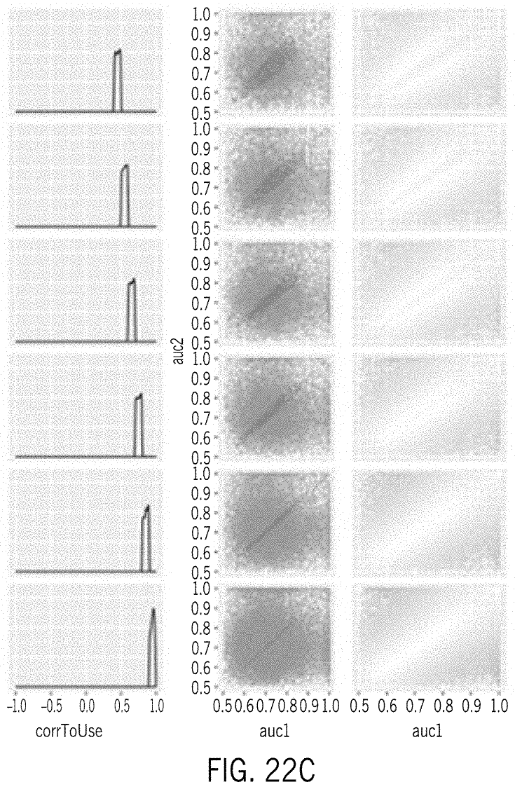

Repeating the analyses above yields very similar result plots (see FIGS. 21 and 22), with the notable difference that, with a similar number of samples as in FIGS. 18 and 19, and examining a similar correlation slice, the boundary between the areas of positive/negative ΔAUROCSF[AB] is much sharper, indicating an increased certainty of whether the resulting fusion is positive. Specifically, we repeated each of the analyses from FIGS. 18 and 19 above, first transforming each scoring system into a ranking system (by sorting score values), and performing rank-based fusion. In both sets of analyses, the boundaries are more clearly defined in the rank fusions than in the score fusion case. Visual comparison of the score fusion and the rank fusion suggests the increased resolution approximates a ten-fold increase in number of observations: the resolution for N=600 with the score function is similar to that of the rank function for N=50. It is noted that the proper equivalent of Pearson correlation for rank data is the Spearman rank correlation, where, as with Pearson a value of 0 is totally uncorrelated, and a value of 1.0 represents perfect positive correlation (and −1.0 perfect negative correlation). However no change in the setup is necessary, because calculating Pearson correlation on numeric ranks yields the Spearman rank correlation directly (i.e., DPC(RSA,RSB)=DSR(RSA,RSB)).

The implication of these results is that the geometry seen in the plots, and by consequence the ability to predict the utility of any given fusion, is a factor of the ranks of the samples, and not of their scores. Indeed, because a potentially infinite number of different scoring systems can yield the same ranking system, it is hypothesized that, for some purposes (and in this case), the results seen here manifest entirely as a factor of ranks, with the move to scores only adding relatively uniform and unbiased noise. For example, this analysis reveals that the interactions between “Accuracy” and “Diversity” are fundamentally rank-based, where an important factor is the ordering imposed by SSA and SSB on (the observations in) C1 and C2.

FIG. 21 shows the success or failure of rank fusions can be more predictable than score fusions. Both panels of FIG. 21 show random sampling of pairwise rank fusions (N=600/class). The x- and y-axis of these plot indicate the performance (in AUROC) of SSA and SSB respectively. The color indicates the net improvement of the fusion AUROC (AUROCRF[AB]), compared to the better of the two input systems AUROCM. In the upper panel of FIG. 21, blue (e.g., the central region) is positive (ΔAUROCRF[AB]>0) and red (e.g., the outer region) is no improvement (ΔAUROCRF[AB]≤0). The lower panel of FIG. 21 shows ΔAUROCRF [AB].

For FIGS. 22A-22C., the columns C, D, E (left to right) show for C, the distribution of correlations in each slice moving from −1.0 to +1.0 in 0.1 intervals (or slices). In particular, column C shows each sub-panel being restricted by the Spearman rank correlation between the fused pair to a 0.1 unit range of window (from −1 to 1). Columns D and E show random sampling of pairwise rank fusions (N=600/class), similar to the panels of FIG. 21, but stratified by correlation.

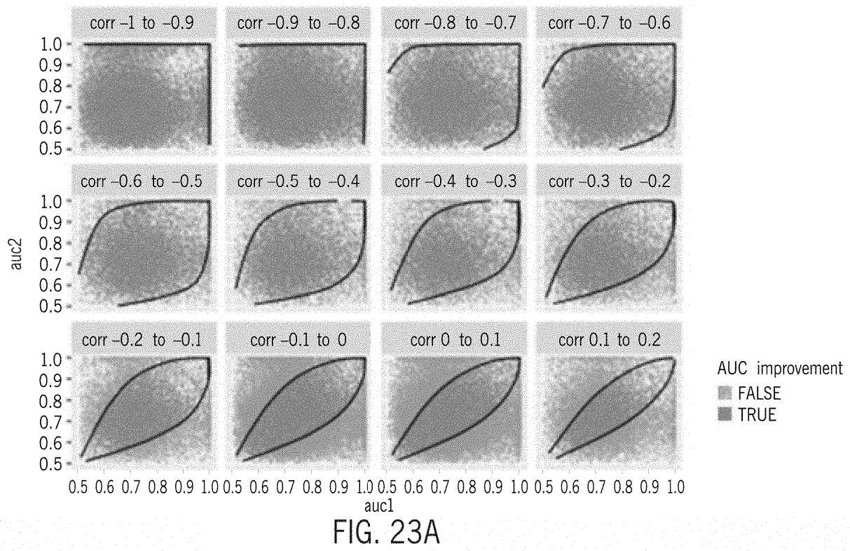

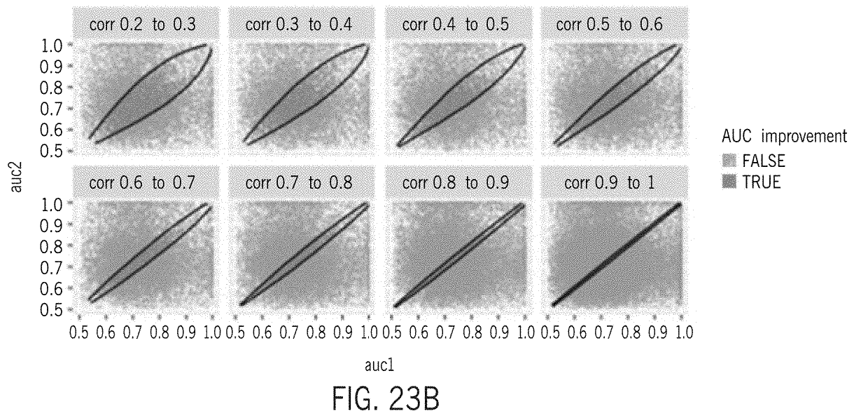

A straightforward method for modelling the boundary between regions where the fusion was a net improvement, and regions where it was not was sought so as to enable accurate prediction of future fusions. As noted above, predicting the outcome of fusions is a fundamentally challenging and an important problem. Collectively, the results above provide a strong indication that there exists a relationship between the AUROCs of two systems to be fused, the between-system correlation of the ranks accorded to the individual observations within each system, and the “N” that went into comprising the systems. Due to the variability of the shape of these regions observed in the simulation studies (detailed above), and aided by the abundance of the simulated data, we elected to model the boundary non-parametrically using locally weighted scatterplot smoothing (“LOWESS”) curves. The LOWESS curves generated from simulated data correctly discriminated the vast majority of positive from the vast majority of negative cases (see, e.g., FIG. 23).

For FIG. 23, the Locally Weighted Scatterplot Smoothing (Lowess) curves accurately capture the boundary in rank fusions of simulated data. The Lowess curves were fit to the rank fusion training data, stratified by correlation into 20 bins (or slices, or correlation intervals). The LOWESS curves were created in R ver 3.5.2, and were trained on the rank fusion data shown in FIGS. 22A-22C.

To test the validity and to demonstrate the utility of the diversity of ranks and accuracy (the “DIRAC” framework), the approach and the specific Lowess curves derived above were used to predict fusion improvement in a realworld dataset. This demonstration used data from the Multiethnic Cohort adiposity phenotype study (“MEC-APS”), a study of adiposity phenotypes in men and women from five ethnic groups. Specifically, we used body fat levels and body fat distribution determined using dual-energy X-ray absorptiometry (“DXA”) and magnetic resonance imaging (“MM”), in 1000 subjects (533 women). Approximately equal numbers of Japanese-Americans, African-Americans, Latino(a)s, Native Hawaiians, and Caucasians were imaged. Overall, DXA imaging is cheaper, and more clinically available, but is less accurate at determining body fat distribution. The accuracy in (AUROC) of each of 31 DXA measurements (e.g., ranking systems) in attempting to predict each of 41 MRI measurements was determined. Then, a pairwise within-class correlation measurement and fusions (in a manner identical to the simulated data experiments above) for all possible pairings of DXA variables was carried out. The ΔAUROCSF[AB] and AUROCSF[AB] of the fused DXA predictor was calculated in an attempt to predict each MM variable.

To test the applicability of the fusion techniques presented in this work to real-world data, MM-based measures of body fat distribution (e.g., liver fat, visceral fat at the L1-L2 vertebral boundary, and the like) were used as the ground truth target variables, and DXA (dual-energy x-ray absorbtiometry) variables that captured general body fat served as the predictor variables to fuse. These data were drawn from the MEC-APS, in which 1861 individuals from the Multiethnic Cohort had their body composition measured by DXA and their abdominal fat distribution assessed by MRI between L1 and L5 between 2013 and 2016. Details on the MEC itself and the imaging study have been published.

Each of the target MRI variables were divided into quantiles (medians, tertiles, quartiles, and quintiles), and it was measured how well each DXA variable was able to discriminate the top quantile from the bottom in terms of AUROC. We then fused a number of possible pairs of DXA predictors, measuring the correlation between the two input scoring systems, and the AUROC of the fused system. This fusion performance was then compared to that of the simulated data.

Three tools (e.g., interactive tools) were created to generate the data used in this study to develop the arguments presented in this work. First, a client/server framework for generating simulated data was created that enabled a detailed exploration of scoring system fusion. This tool enabled the construction and analysis of simulated scoring systems using any combination of Gaussian and exponential sampling distributions (either individual scoring systems, or mixtures of different systems, with parameters sampled according to specified sampling distributions). By adjusting the pointwise pairing in the fusion step, the effect of scoring system correlation could be studied, and by adjusting the number of samples drawn from each set of parameterized distributions the effect of the sample size (N) could be studied.

Second a web-based python/javascript interactive tool was developed to allow detailed exploration of input data distributions and the effects of fusion, using either simulated data from the simulation framework described, or real data originating from other studies. This tool enables the user to examine the overall distribution of fusion points within the AUROC input space, filtered by correlation coefficient (e.g., different correlation intervals or slices). Within a given fusion, the user can examine the case/control distributions using histograms and scatterplots, and the overall scoring system AUROC curves, and examine exactly how the mechanics of additive fusion change the fused scoring system distributions, for better or worse. Thirdl, a simple LOWESS model was created by fitting these curves to the simulated rank fusion data for each correlation interval (each correlation interval having pairs of data models plotted). The LOWESS model can estimate the chance (e.g., by providing a specific threshold) that a fused scoring system will perform better that the higher-performing of the inputs given estimates of the input AUROCs and the correlation between them.

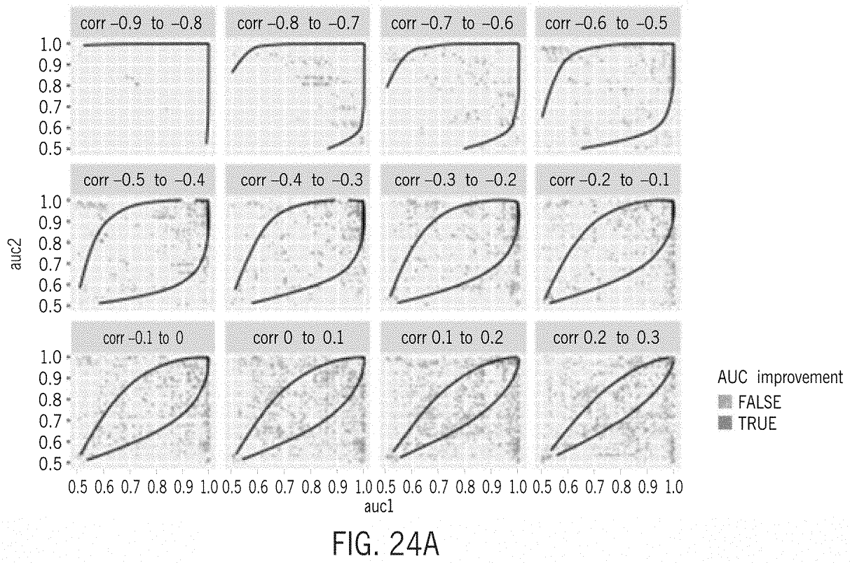

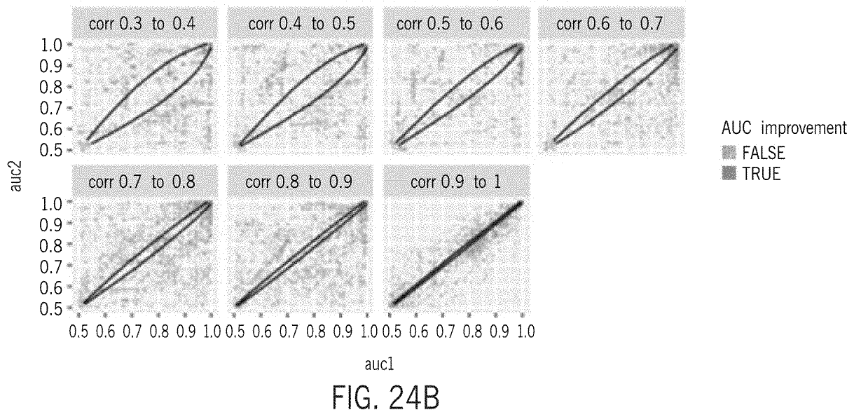

The resultant fusion of DXA variables to predict MRI variables was captured with near perfection by the LOWESS curves trained on simulated data (see, e.g., FIGS. 24A and 24B). Superimposing the LOWESS curves generated from the simulated data onto this real-world DXA-MRI prediction data reveals that the shape of the regions of fusion improvement in both are very similar. Visual inspection suggests that an accuracy is x>99.7% (x<300 missed—negative fusions inside the LOWESS curves or positive fusions outside the curves in the 114,390 calls). Analysis also suggested that the noisiness of the transition zone between positive and negative ΔAUROCSF[AB] is comparable to that seen using a simulated data set of a similar size (N). It is believed that those cases in which the LOWESS curves failed to predict outcome accurately may result from the use of a relatively coarse correlation slice (e.g., each of the 20 panels shown represents a 0.1 unit range of r). Thus, moving to, for example, 200 “panels”/correlation slices would makes the steps in r 0.01, and would reduce some “borderline” errors) and potentially for relatively small numbers of simulated points at the highest AUROC values.

FIGS. 24A and 24B shows Lowess curves that were built on simulated data, accurately capture the boundary in real world rank fusions. For example, as shown, fusions of DXA data can reasonably predict MM outcomes and are plotted in the same way as simulated data, with Lowess curves, for each correlation group that was previously determined by the simulation data, superimposed on the corresponding correlation group for the DXA fusion data

These data from the MEC-APS study demonstrate how fusing predictors (e.g., two DXA measurements on any given individual) can improve the quantile prediction accuracy of a given ground truth MRI variable, and how we can predict whether the fusion will result in a net increase in performance, knowing only the AUROCCs of the two input DXA measurements, and the correlation between their ranking systems.

This method also opens the door to identifying unexpected relationships for follow-up. As a specific example, we note that the analysis of potentially beneficial fusions suggests that visceral fat predictions made by DXA imaging-derived markers of fat are predicted by DIRAC (e.g., the above approach) to increase in accuracy when one considers the bone mineral density (“BMD”) measures as well. This prediction, which was unexpected biologically, was confirmed experimentally, and this result has led us to re-examine the literature that potentially links bone mass and visceral fat.

One reason for combining two scoring systems is to achieve a level of accuracy that is higher than either of the systems alone. The current approach has shown that, for a particular type of combination, the performance can be predicted in advance with high precision, knowing only the accuracy of the input systems, and the within-class correlation between them. The dependence of this result on the size of the dataset was explored, and it was shown that, though systems may be combined at the score level or at the rank level, rank level fusion predictions are more accurate. This difference is especially noticeable at smaller sample sizes.

It is hypothesized that the difference in prediction precision between score- and rank-based fusions results from the fact that the structure and geometric regularity that are observed in system fusion originates largely from the rank level. The reason for the difference in precision between score and rank fusions may then be explained by the potentially infinite number of scoring systems that map to a single ranking. Coupled with the fact that the AUROC is an inherently rank-based metric, we posit that the additional information in the score distributions themselves is not properly accommodated in the simple fusion framework we describe, and manifests instead in the additional noise observed at the score fusion decision boundary.

The distributions of scores, or functions involving both scores and rankings, may represent domain-specific information that may be exploited for additional performance in certain situations, though the no-free-lunch theorems preclude this from being true generally. One important point to note is that if fusion prediction relies only on the rankings of the input systems, then there is no longer any connection to the original score distributions. This means that the results that are presented are, by definition, general and domain-independent—the only restriction is that the input systems be monotonically increasing scoring functions.

Whether the fusion involves scores or ranks, the geometric structures visualized in the figures makes explicit the relationship between Accuracy and Diversity in this particular formulation of classifier system fusion. Accuracy and Diversity have long been hypothesized to be of fundamental importance to fusion in general, but the relationship has been poorly understood. The characterization, as described above, has confirmed the intuition that improvements in accuracy more readily occur when fusing two uncorrelated systems (e.g., with a within-class correlation near zero), but has further revealed the more surprising reality that negatively correlated system pairs are even more likely to produce a fusion with an increased accuracy, and that the improvement may be even greater, boosting even marginal classification systems to higher (and occasionally very good) performance.

Provided both systems have some predictive value (e.g., AUROC>0.5) then a strongly negative correlation can allow an already accurate system to fuse beneficially with a poor one. In the limiting case, a nearly perfect negative correlation between two systems that are only a hair's breadth better than random chance, can fuse toward perfection (e.g., AUROC=1.0). It is noted that in this formulation, the systems were oriented so as to have a predictive accuracy such that AUROC>0.5. Data in the figures clearly make explicit that it can be more beneficial to choose two less accurate systems to fuse than two more accurate systems, if and only if the correlation is lower for the former pair than for the latter. Consider that, conceptually, highly correlated systems are typically understood to be representative of the same latent signal in the data with respect to the target outcome, and uncorrelated systems representative of different latent signals with respect to the target outcome. Thus, in this interpretation, negatively correlated systems can be understood to represent complementary latent signals, with respect to the particular target on which the performance metric is based. In this case, that target is an ordering of the samples in the dataset that perfectly separates both classes—*an* ordering, because there are inherently many (NC1!*NC2!/2) such orderings that have the same AUROC. By calculating the average within-class correlation as described, rather than the global correlation, the target is implicitly taken into account, and the within-class correlation metric then directly quantifies the complementarity of systems with respect to the target.

There are several ways in which the findings may be directly useful in domains where system fusion is potentially applicable (e.g., a domain in which multiple scoring systems can be constructed). First, the results provide strong, quantitative support that choosing (or constructing) system pairs that are maximally uncorrelated is likely to be a beneficial strategy, and the data presented quantitatively illuminates the extents to which this intuition applies in practice. This includes the demonstration of the specific potential utility of fusing systems that feature negative within-class correlation. These results suggest that a beneficial approach, when constructing classification systems, may be to select component pieces that have a high a-priori likelihood of being uncorrelated or negatively correlated (and therefore complementary). One approach might be to combine systems built on unrelated or inversely related sub-domains of the problem at hand—for example, fusing a model built on gene expression data with one built on categorical environmental variables. Another approach might involve the fusion of two very different statistical models built on the same dataset—fusing, for instance, a system based on logistic regression with a rule-based classifier system, or fusing a system that identifies single strong variables (e.g. the least absolute shrinkage and selection operator “LASSO”) with one that distributes predictive power (e.g. projection methods).

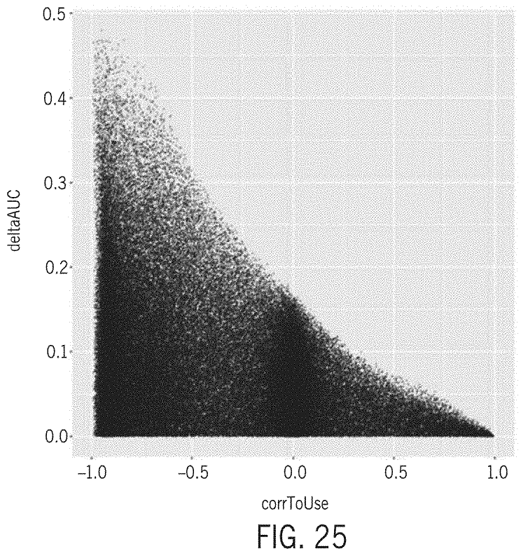

Second, this approach provides a direct test of whether an inferior model can be successfully fused with a more accurate model. Historically, it was known that including a less capable classifier system in a fusion can sometimes boost overall accuracy, but the reasons why Diversity matters were unclear and the resulting performance was only weakly predictable at best. With the relationship between Accuracy and Diversity quantitatively determined in this framework, a targeted, iterative approach can be taken for classifier system construction. With the accuracy of an initial system assessed in a test population, this framework indicates the combination of within-class correlation and AUROC that a second system need have for the fusion of these two systems to outperform either alone. Perhaps more importantly, it establishes hard boundaries below which fusing a second, less accurate system is very unlikely to help (e.g., FIG. 25).

Third, this framework reveals that fusion improvement can be predicted accurately with only the three quantities measured. This directly indicates that estimates of these quantities may be obtained separately from each other in space or in time, provided the populations in which they are determined are sufficiently similar. This is a potential advantage when attempting to integrate the results of disparate previous studies, or when selecting previously tested systems to include in a fusion system under construction.

Fourth, this framework implies that any two classification systems yielding equally accurate models that are highly correlated have essentially equivalent utility. This means that the easier or cheaper option may be selected, without compromising the overall accuracy of the fused system.

Fifth, because this framework is inherently domain independent, it is expected to be applicable in far-ranging areas, For example some areas can include clinical biomarker development/personalized medicine (e.g., to determine whether combinations of specific markers can be beneficial, optimize information gain relative to costs, integrate multiple information streams such as clinical chemistry and clinical phenotypes), clinical trial enrollment (e.g., optimize enrollment of informative subjects), insurance pricing (e.g., to leverage distinct information streams about potential risks), portfolio management (e.g., multiple predictors can be joined to maximally leverage information, to balance gain and potential risk, and the like), and sensor optimization.

As noted above, rank fusions are more predictable than score fusions, especially at lower numbers (e.g., of N). Despite the loss of information inherent in converting scores to ranks, many classification problems are well-represented by sample rankings. These include such “top-N/bottom-N” problems as separating the top and bottom quintiles of a population in terms of disease risk, or selecting the 10 best performing stocks for a portfolio. Often these types of problems interface with an external constraint. For example, if a research organization only has enough money to enroll 100 patients in a clinical trial, what is important is selecting the 100 best candidates to enroll. Of less importance is the exact numerical difference between the 100th and the 101st candidates. Instead of constructing full predictive models of classifier score distribution, then establishing a numerical threshold for identifying the “top-N” samples, the ranking system fusion approach, applied as described in the DIRAC framework, suggests a method for model construction using the rankings of the samples directly.

Score fusions may have distinct utility in other problems. The noise near the ΔAUROCSF[AB]=0 is greater in score than rank fusions, but there is still plenty of area where our predictions of score fusion accuracy have high precision, and the absolute gain in accuracy in score fusions can be higher than in rank fusions (see., e.g., FIGS. 18, 19 vs. FIGS. 21 and 22). Additionally, discriminative models that establish numerical classification bounds (e.g., a disease threshold) that are uncoupled from the population of training samples may be constructed using score fusions, but not with rank fusions. It is hypothesized that score-based fusion approaches are likely to be more domain-dependent. Making use of the extra information present in the score distributions and combining those approaches into this framework may be another area of future interest.

This framework opens multiple avenues for future research into both the mathematical extensions and underpinnings of the observations presented in this work and its practical applications. Due to the number of possible ways to combine systems, and the number of available metrics for measuring both system accuracy and system diversity, this approach was necessarily limited to a subset of these approaches and metrics. Here we focused on one fusion approach (average fusion) and one commonly used set of metrics, the AUROC for measuring accuracy, and Pearson's correlation for measuring Diversity in score fusions (and rank fusions, such as with using Spearman's correlation). In addition to their specific utility in some applications, these metrics are well understood, generally applicable, and popular. It should be understood that a very large number of fusion methods can be constructed—arithmetic, geometric, exponential, weighted, and so on. Similarly, the current approach was restricted to considering only fusions between pairs of classifier systems. However, the restriction to pairwise fusions does not preclude the fusions of more than two systems within this framework, but it does restrict the construction of a fused system to iterative pairwise construction, similar in practice to forward stepwise regression. The sequential and/or simultaneous fusion of multiple scoring (at least >2) systems is another promising area.

Some non-limiting examples of the disclosure, as described above, provide a geometric (or theoretical) approach. The previous evidence described above, has largely been derived from empirical experimentation, but utilizing the structure above, a theoretical explanation can be provided for the results observed thus far. Rankings are more than a list of numbers, such as a list of scores from a scoring system, or the normalized versions thereof. They are permutations of the set of natural numbers of size N (here, the number of samples/observations in a dataset), and have been well studied in the fields of combinatorics and group theory—in particular the theory of symmetric groups. Symmetric groups have a regular geometric structure of dimension N−1, which forms a convex polytope embedded within the larger N dimensions of the total space of the samples. Various convex polytopes, or permutahedra, can be displayed in three dimensions without projection, thus retaining their symmetry. For example, a permutation group containing two elements (called S2) is a line. As another example, a permutation group containing three elements (called S3) is a hexagon. As yet another example, a permutation group containing three elements (called S4) is a truncated octahedron. The vertices of these polytopes represent all the possible permutations of the elements of which they are constituted, and the edges represent the adjacent transpositions that are necessary to move from one permutation to all the possible adjacent permutations.

It is proposed that the regular geometry that is observed in pairwise combinations of ranking systems of N elements may be explained by considerations of the geometry of the corresponding S_{N} polytope. Evidence for this proposal is provided by constructing a representation of distance across the surface of the permutation polytope using angles in a vector space corresponding to an N−1-sphere (a hypersphere embedded in the space of the ranking systems (rank space/sample space)). It is shown that two representations of diversity are possible, corresponding to two different angles in this framework, and that a similar ellipsoid geometry of fusion improvement is observed when this distance metric is used in place of the Spearman correlation distance. It is also shown that the mean fusion of two systems is equivalent to finding the ranking system vertex closest to the centrepoint of a geodesic arc across the surface of this N−1 dimensional polytope, connecting the two ranking systems being fused.

The construction of an angle-based representation of distance requires the establishment of a suitable origin located at the barycenter of the permutahedron. For convenience and without loss of generality the entire structure is translated so that its barycenter is located at [0, 0, . . . , 0], which is achieved by subtracting the mean of the ranking system from each of its elements—the mean of each ranking system is the same, as they are simply permutations of the same set of elements (the natural numbers 1 to N). For example, a ranking system that placed each element in a strictly increasing order [1, 2, 3, . . . , N] with N being odd would have mean:

$$M = ( {\sum }_{k = 1}^{k = N} k ) / N$$

and thus would have sequence elements [1−M, 2−M, . . . , 0, . . . , N−M]. For N being even there would be no center element of 0. From this origin point of [0, 0, . . . , 0] it is apparent that the vertices representing the ranking systems lie on a hyperspherical manifold for the same reason that each ranking system has the same mean value—the vector coordinates of each ranking system are simply permutations of each other, and therefore have the same L2 norm (e.g., ∥x∥²=\sqrt{x₁²+ . . . +xₙ²}) meaning that they are located at the same Euclidean distance from the origin/barycentre point. This allows a simple calculation of the involved angles as inverse-cosines of their appropriately scaled dot products. For two ranking systems x and y (post-translation) the angle θ between them is given by θ=arccos((x*y)/(∥x∥*∥y∥)).

Under this framework, the fusion of two ranking systems may be viewed as an inherently spherical problem involving three points only—the target point representing the ideal target sequence, and the two input system points that are desired to be potentially fused. Because the target point can be fixed at the pole, the angles that are used can be calculated to locate these points without reference to their N-dimensional coordinates. In this instantiation, pairwise fusion becomes a 3-dimensional spherical problem, and may thus be visualized as a sphere (e.g., a hemisphere).

This spherical framework allows pairwise fusion to be represented as spherical triangles, where their relationship may be analyzed using spherical trigonometry identities. The “performance” of the two input systems to fuse is represented by the angles p₁ and p₂ between the target point at the pole of the sphere, and the systems SS₁ and SS₂ respectively. Two possible formulations of the “diversity” are also apparent. There is the angle which represents the shortest geodesic path separating the two input scoring systems, and there is also the angle, which represents the rotation around an axis connecting the origin with the target point. It is proposed that this latter angle is what the “within-class” correlation in the previous work was approximating. The “within-class” correlation was selected to reflect the diversity between the input ranking systems in a way that corrected for their mutual difference in performance. By using the surface angle we “project out” any SS_{1}-SS_{2} diversity that has any association with the performance.

To explore the suitability of these N-sphere angles as “performance” and “diversity” measures, and to compare the differences between the two possible measures of “diversity” that this construction enables, the same series of pairwise fusions were performed, this time using a pair of angles to measure the performance of the input systems, and other angles to measure the diversity between them. The pairwise fusion series using the corresponding angle resembles its Spearman correlation equivalent very closely, lending support to the idea that “within-class” correlation is inherently approximating this angle.

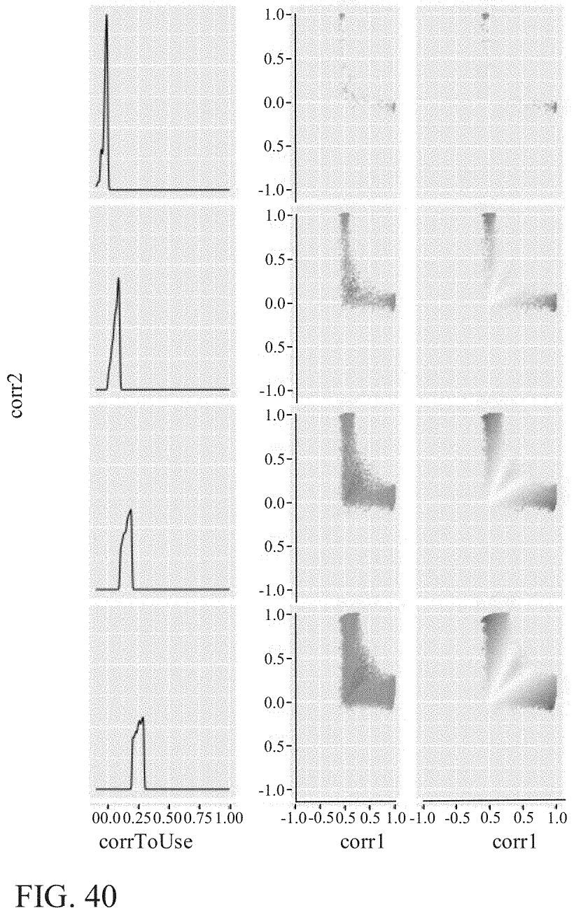

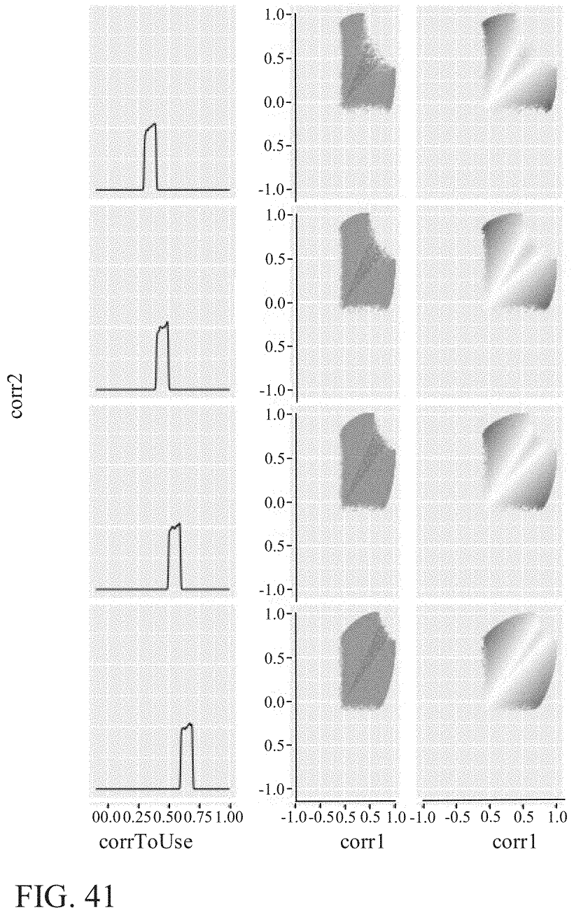

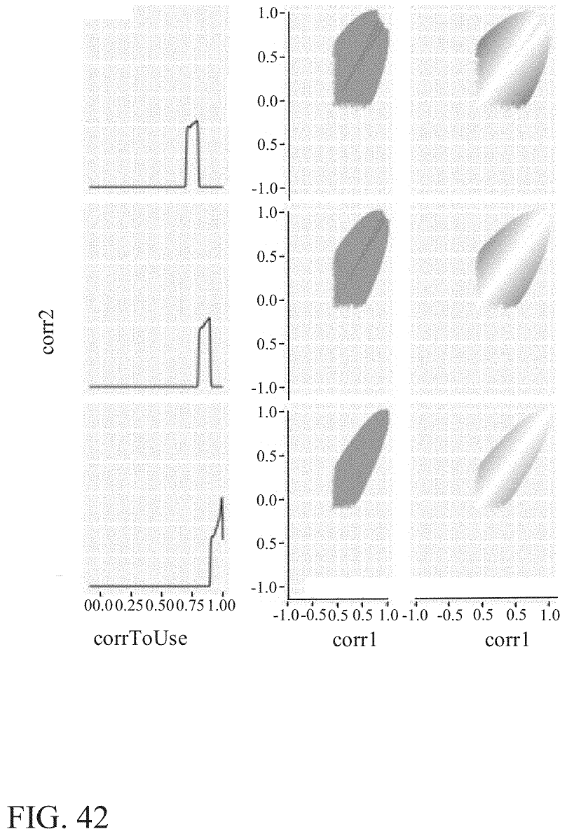

The fusion ellipse plots of FIGS. 26-28 are of continuous scoring systems (e.g., similar to those shown in FIGS. 40-42), where angle1 and angle2 are the angles that the two input scoring systems make with the single, “perfect” target point (e.g., a suitable origin located at the barycenter of the permutahedron). The black boundaries drawn on top of the data points are derived directly from the spherical permutahedron theory, as described above. As indicated, the upper plots are based on “global” correlation (the angle directly between the two scoring systems), and the lower plots are based on the usual “within-class” correlation (or rather the continuous equivalent of it, which was theorized as the rotation angle around the single “perfect” point—pure diversity flips). As shown, discrimination can be made equally well in both cases. For FIGS. 26-28 no “negative” correlations are shown (in these angle cases that means “global” or rotation angles greater that pi/2).

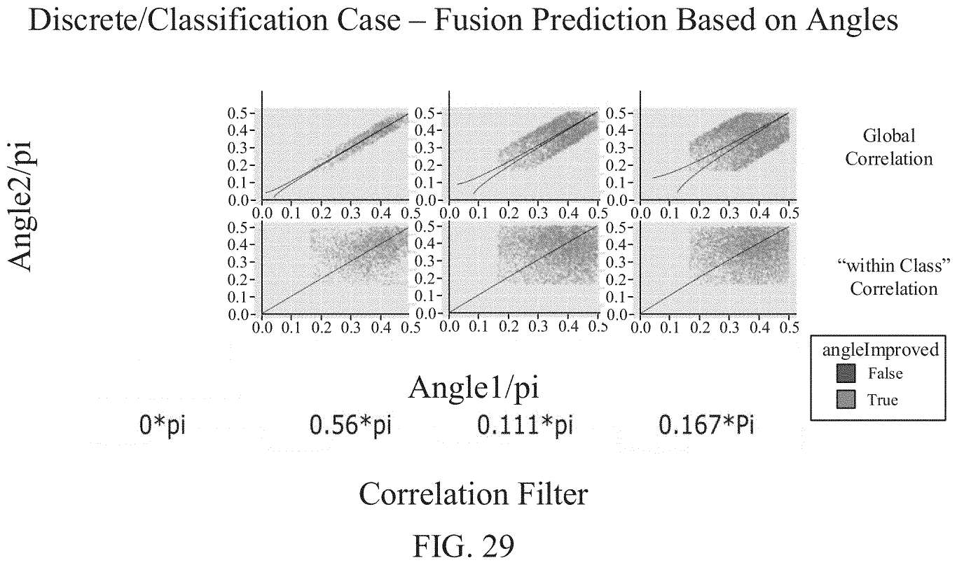

The discrete (quantile/case-ctrl) plots of FIGS. 29-31 use the angle between the scoring system and the interior centroid point representing AUC=1, instead of AUC directly. The aucAngle is the angle that exists between the vector that represents any given scoring system, and the vector that represents the AUC=1 interior centroid point. This point is the centroid of all possible permutations which perfectly separate the two classes. This point is trivially easy to construct—in the S4 case (where [1, 2, 3, 4] is the “perfect” sequence, with 1, 2 being “case” and 3, 4 being “control”, the interior centroid point representing AUC=1 is [1.5, 1.5, 3.5, 3.5.]. If information equivalent to the AUC can be measured using angles, then it is possible to use the same angle-based framework in both the continuous and discrete cases. This would provide strong evidence that the theoretical framework is at the foundation of everything detailed so far. Below are plotted simulated data, showing the aucAngle instead of the plain AUC. As before, the upper plot in each of FIGS. 29-31 is a “global” correlation slice, with global correlation derived discrimination bounds, and the lower plot is our standard “within-class” correlation case. The curves superimposed on the data are from the exact same theoretical derivation shown above in the continuous case. One interesting thing to note is that the aucAngle never reaches zero—there is a lower limit as a ranking can never contain the same values as an interior point.

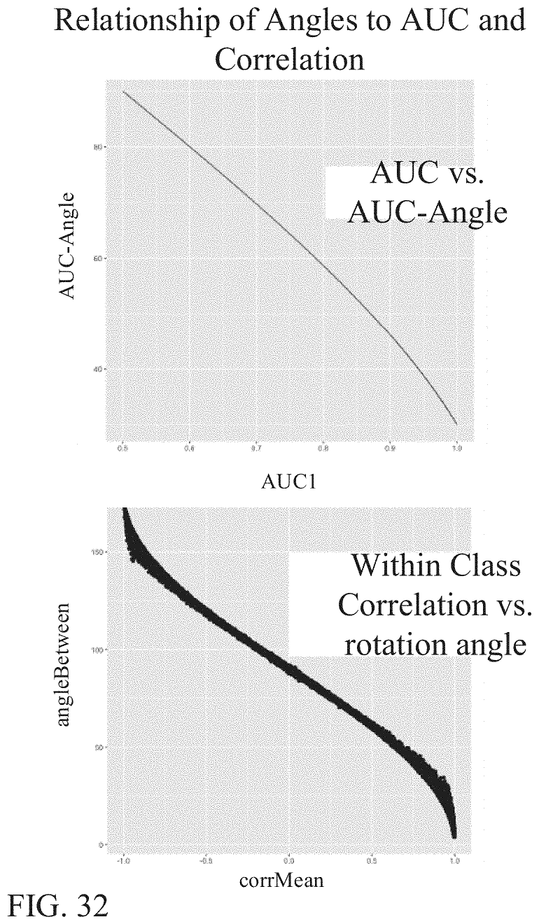

FIG. 32 show the discrete case angle comparison AUC vs aucAngle. Here AUC (as we used before) is compared with aucAngle. As shown, these are illustrating the exact same features as above.

FIG. 32 also show the within-class correlation vs rotation angle. Here the within-class correlation that we've always used is compared with the angle of rotation about the AUC=1 centroid vector. As shown, again the same features are illustrated, but the rotation angle does a better job at discriminating (e.g., see for example the ellipse boundaries). This provides evidence that the rotation angle is diversity, geometrically.

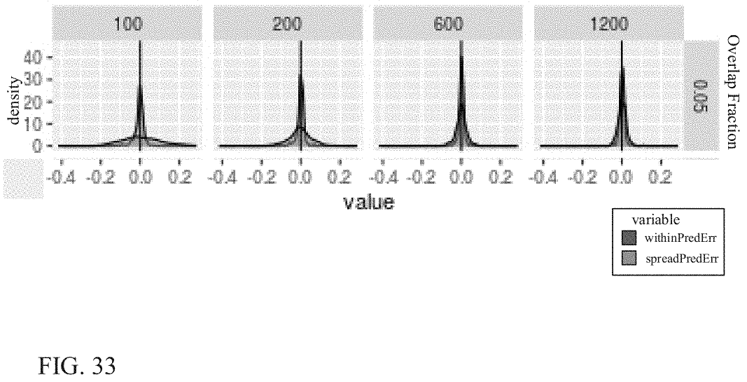

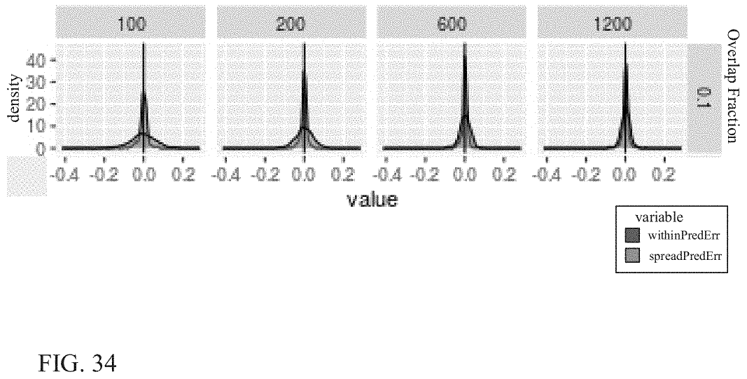

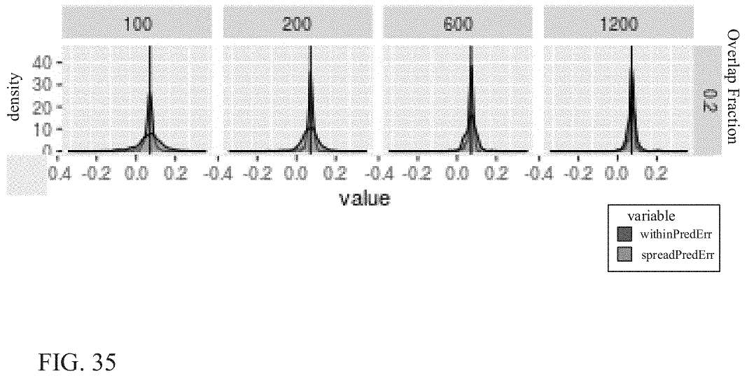

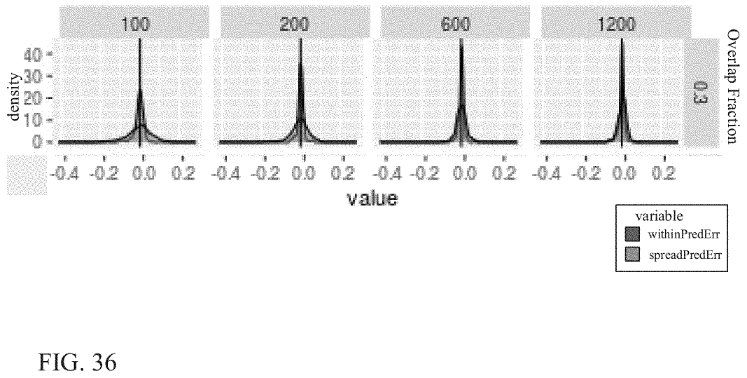

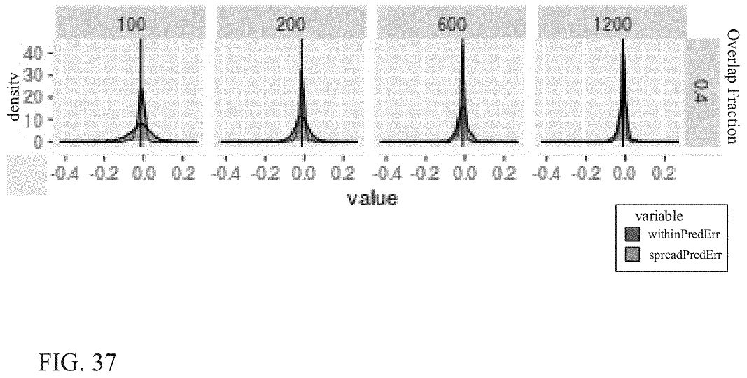

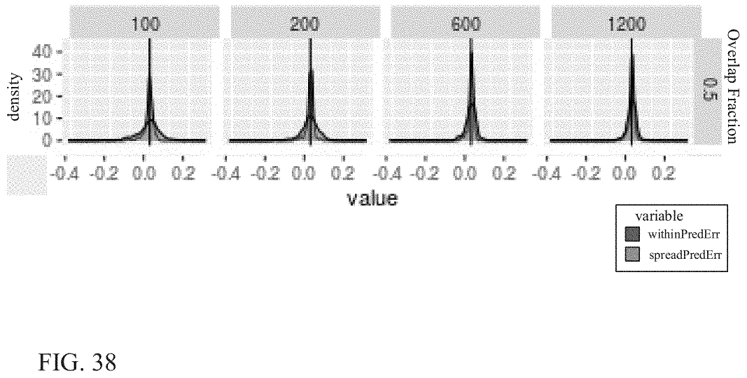

To explore a different example of the continuous case (rather than the discrete case), a population was determined, which in this case was a simulation. However, it is noted that although this was a simulated population, this is still equivalent (or applicable) to anything that is considered a valid population for a given study (e.g., all men over age 20 and under age 40 in the United States), where the two models are tested on essentially independent series, with a partial overlap used to obtain the correlation. FIGS. 33-39 show the results of the analyses performed on overlapping and non-overlapping subsamples, all drawn from the same original dataset (e.g., a simulated population, although other equivalent populations could be used). The data simulation experiments proceed as they have always done, and this ends up producing a simulated dataset of a given size, that has two scoring systems (to fuse). This single dataset is divided up into two equal parts, called group A and group B. Then an equal number (equal to or smaller than the size of group A/B) of points are randomly selected from both A and B, and we “copy” them into another group called groupOverlap. Then, focusing on group B, three quantities are computed. Frist, the fusion accuracy within group B is determined by explicitly performing the fusion within this group and measuring its accuracy. Second, the fusion accuracy in group B is estimated using a loess based model trained on previously generated data with N=600, by feeding into the loess model the auc1, auc2, and within class correlation, all measured within group B. Third, the same model to estimate the fusion accuracy of group B is used, but this time the auc1 measurement from group A, the auc2 measurement from group B, and the within-class correlation from groupOverlap are used. This can show at least three things. One, what is the baseline “real” fusion accuracy in group B? Two, how accurately can we estimate this fusion using the model? And three, how accurately can we estimate this fusion using the model and measuring the three quantities in three subsets of the data?

Each graph of FIGS. 33-39 represents a particular total dataset size (the N of the experiment, denoted as 100, 200, 600, and 1200) in the columns, and a particular overlap fraction (e.g., the fraction by which the “overlap” group overlaps each of the two groups A and B) in the rows. The baseline performance (quantity 1 above—the explicit fusion accuracy within the group) is represented by a vertical line at 0.0, and then the two estimation errors are shown as density plots on top of this line—the red distribution (“withinPredErr”) represents quantity 2 above (the estimated fusion accuracy within the group), and the blue distribution (“spreadPredErr”) represents quantity 3 above (the other estimated fusion accuracy within the group).

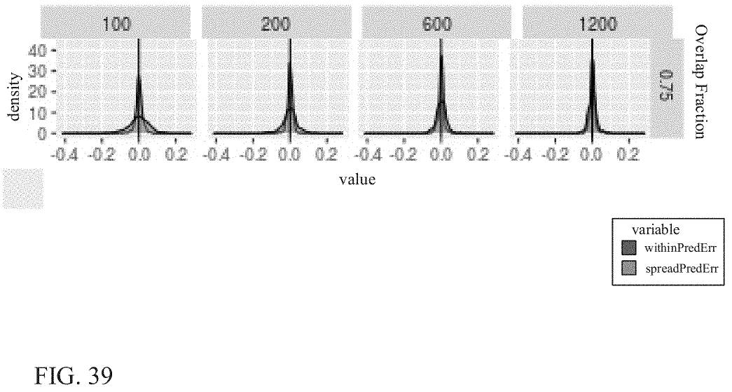

The complete overlap in the left graph of FIG. 39 shows that, for example, with 300 people per group of whom 90 overlap there is no difference in prediction accuracy (e.g., models can be built and tested separately and, as long as there is enough overlap (including an essentially synthetic group) to test correlation the signal is not lost). The far right (e.g., the last right hand) graph of FIG. 33 shows that, with N's of 600/group and with 30 people overlapping, there is essentially no loss in accuracy. It is noted that the overall predicative accuracy is a little lower in this series because the specific LOWESS modeling algorithm used here is a little less accurate.

As noted above these are continuous fusions, using Spearman rank correlation to measure the performance (e.g., this replaces AUC), and also to measure the diversity between points (e.g., correlation slices, as done for the classification case). Using the Spearman rank correlation, you cannot get a target-reflecting correlation quantity such as within-class correlation, or surface angle, so the SRC diversity distance really corresponds to the “global” correlation in the continuous case. This is a reason why these plots look different to others.

It is important to note that the size of the overlap appears to be only important at mid/small dataset sizes, and once the N gets large enough, the auc1/auc2/within-class-corr values in each subset appear to be always quite close to each other. This does show that estimating the auc1/auc2/within-class-corr in separate subpopulations works (and suggests that three completely separate populations are also likely to work). It is important to note that in FIGS. 33-39 how the red and the blue distribution overlap. At relatively high N and overlap values, the two distributions are essentially the same, and thus it appears that all the error is coming from the modeling. There does not appear to be any detectable extra error coming from the subsampling.

Although these systems and methods has been described and illustrated in the foregoing illustrative non-limiting examples, it is understood that the present disclosure has been made only by way of example, and that numerous changes in the details of implementation of these systems and methods can be made without departing from the spirit and scope of these systems and methods, which is limited only by the claims that follow. Features of the disclosed non-limiting examples can be combined and rearranged in various ways.

Furthermore, the non-limiting examples of the disclosure provided herein are not limited in application to the details of construction and the arrangement of components set forth in the following description or illustrated in the following drawings. These systems and methods is capable of other non-limiting examples and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

Also, the use the phraseology and terminology used herein is for the purpose of description and should not be regarded as limiting. The use of “right”, “left”, “front”, “back”, “upper”, “lower”, “above”, “below”, “top”, or “bottom” and variations thereof herein is for the purpose of description and should not be regarded as limiting. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

Unless otherwise specified or limited, phrases similar to “at least one of A, B, and C,” “one or more of A, B, and C,” and the like, are meant to indicate A, or B, or C, or any combination of A, B, and/or C, including combinations with multiple or single instances of A, B, and/or C.

In some non-limiting examples, aspects of the present disclosure, including computerized implementations of methods, can be implemented as a system, method, apparatus, or article of manufacture using standard programming or engineering techniques to produce software, firmware, hardware, or any combination thereof to control a processor device, a computer (e.g., a processor device operatively coupled to a memory), or another electronically operated controller to implement aspects detailed herein. Accordingly, for example, non-limiting examples of these systems and methods can be implemented as a set of instructions, tangibly embodied on a non-transitory computer-readable media, such that a processor device can implement the instructions based upon reading the instructions from the computer-readable media. Some non-limiting examples of these systems and methods can include (or utilize) a device such as an automation device, a special purpose or general purpose computer including various computer hardware, software, firmware, and so on, consistent with the discussion below.

The term “article of manufacture” as used herein is intended to encompass a computer program accessible from any computer-readable device, carrier (e.g., non-transitory signals), or media (e.g., non-transitory media). For example, computer-readable media can include but are not limited to magnetic storage devices (e.g., hard disk, floppy disk, magnetic strips, and so on), optical disks (e.g., compact disk (CD), digital versatile disk (DVD), and so on), smart cards, and flash memory devices (e.g., card, stick, and so on). Additionally, it should be appreciated that a carrier wave can be employed to carry computer-readable electronic data such as those used in transmitting and receiving electronic mail or in accessing a network such as the Internet or a local area network (LAN). Those skilled in the art will recognize many modifications may be made to these configurations without departing from the scope or spirit of the claimed subject matter.

Certain operations of methods according to these systems and methods, or of systems executing those methods, may be represented schematically in the FIGS. or otherwise discussed herein. Unless otherwise specified or limited, representation in the FIGS. of particular operations in particular spatial order may not necessarily require those operations to be executed in a particular sequence corresponding to the particular spatial order. Correspondingly, certain operations represented in the FIGS., or otherwise disclosed herein, can be executed in different orders than are expressly illustrated or described, as appropriate for particular non-limiting examples of these systems and methods. Further, in some non-limiting examples, certain operations can be executed in parallel, including by dedicated parallel processing devices, or separate computing devices configured to interoperate as part of a large system.

As used herein in the context of computer implementation, unless otherwise specified or limited, the terms “component,” “system,” “module,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

As used herein, the term, “controller” and “processor” and “computer” include any device capable of executing a computer program, or any device that includes logic gates configured to execute the described functionality. For example, this may include a processor, a microcontroller, a field-programmable gate array, a programmable logic controller, and the like. As another example, these terms may include one or more processors and memories and/or one or more programmable hardware elements, such as any of types of processors, CPUs, microcontrollers, digital signal processors, or other devices capable of executing software instructions.

Although the description above, with regard to the processes above, has been framed with respect to specific computing devices implementing these processes (as appropriate), it is also understood that a non-transitory computer-readable medium (e.g., such as the article of manufacture described above) can store computer-executable code for the processes described above. For example, processes 200, 300 (or others) can be effectively stored on the non-transitory computer-readable medium.

As described above, the methods and processes of the disclosure can be implemented on computing devices including, for example, a server (e.g., the server 106). While cloud-based systems offer some advantages in the modern world, it is equally clear that local systems (e.g., the computing device implementing some or all of the disclosed processes or methods), such as point of care devices, specific on-board computer systems, and the like, have critical advantages. For example, embedded processors save time and energy in avoiding the need to upload and download data and results, and they avoid the potential concern related to a loss of communication at a critical time. They also inherently increase security and privacy (e.g., decrease security and loss of privacy risks). Embedded systems thus offer the potential of more rapid response in situations such as manufacturing (e.g., monitor yield, temperature, pressure, etc) or medical (e.g., measure blood pressure, pulse, oxygen, intracerebral pressure, and conduct instantaneous action (e.g., drug delivery, notify staff, and the like).

1. A system for monitoring a plurality of patients, the system comprising:
a processor device;
a display in communication with the processor device;
a first sensor in communication with the processor device, the first sensor being at least one of:
an electrocardiogram sensor;
a pressure sensor;
a blood oxygenation sensor;
an image sensor;
an impedance sensor; or
a physiological sensor; and
a second sensor in communication with the processor device, the second sensor being a physiological sensor;
wherein the processor device is configured to:
receive, using the first sensor and the second sensor, a first data model being representative of a first class and a second class, the first data model configured to predict a first characteristic that is indicative of either of the first class or the second class;
receive a second data model being representative of a first class and a second class, the first data model configured to predict a first characteristic that is indicative of either of the first class or the second class;
determine or retrieve a first accuracy of the first data model;
determine or retrieve a second accuracy of the second data model;
determine a first correlation between the first data model and the second data model for the first class;
determine a second correlation between the first data model and the second data model for the second class;
utilize the first accuracy, the second accuracy, the first correlation, and the second correlation to determine a recommendation for fusing the first data model with the second data model; and
based on the recommendation being for or against fusion of the first data model with the second data model at least one of:
fuse the first data model with the second data model; or
adjust an operation of the patient monitoring system.

2. The system of claim 1, wherein the recommendation is against fusion of the first data model and the second data model, and
wherein adjust an operation of the patient monitoring system includes the processor device being further configured to prevent data acquisition from the first sensor or the second sensor, based on the recommendation against fusion of the first data model and the second data model for a period of time.

3. The system of claim 2, wherein the period of time includes any time that the patient monitoring system is in operation after the operation is adjusted.

4. The system of claim 1, wherein the first data model includes a first variable that is extracted from data acquired by the first sensor,
wherein the second data model includes a second variable that is extracted from data acquired by the second sensor,
wherein the recommendation is against fusion of the first data model and the second data model, and
wherein adjust an operation of the patient monitoring system includes the processor device being further configured to prevent further extraction of at least one of:
the first variable from further data acquired by the first sensor; or
the second variable from further data acquired by the second sensor.

5. The system of claim 1, wherein the recommendation is for fusion of the first data model and the second data model, and wherein the processor device is further configured to, based on the recommendation for fusion of the first data model with the second data model:
fuse the first data model and the second data model together;
prevent, for a period of time, utilization of the first data model; and
prevent, for another period of time, utilization of the second data model, and
wherein the period of time and the another period of time includes any time that the patient monitoring system is in operation, after implementation of the prevention of the respective utilizations.

6. The system of claim 5, wherein the processor device is further configured to receive a user input indicative of at least one of allowing for the utilization of the first data model, allowing for the utilization of the second data model.

7. The system of claim 1, wherein the computing device is further configured to:
combine the first correlation and the second correlation to determine a combined correlation;
receive an accuracy threshold based on the combined correlation;
compare the first accuracy and the second accuracy to the accuracy threshold; and
based on the comparison of the first accuracy and the second accuracy to the accuracy threshold determine the recommendation.

8. The system of claim 7, wherein the accuracy threshold is a curve that corresponds with the combined correlation, the curve defining a first region and a second region, the first region defining an indication for fusion of the first and second data models, and the second region defining an indication against fusion of the first and second data models, and
wherein the computing device is further configured to:
associate the first and second accuracies with the curve to determine if the first and second accuracies are located in the first region or the second region; and
based on the first and second accuracies being located in the first region, provide the recommendation for fusion of the first data model with the second data model.

9. The system of claim 7, wherein the accuracy threshold includes a plurality of accuracy ranges for a plurality of combinations of accuracies, and
wherein the computing device is further configured to:
utilize one of the first accuracy, or the second accuracy, or both are used to generate a specific accuracy range from the plurality of accuracy ranges; and
based on the first accuracy and the second accuracy being within the specific range, provide the recommendation for fusion of the first data model with the second data model.

10. The system of claim 1, wherein the first class is indicative of a physiological condition of a subject, and the second class is indicative of not the physiological condition of the subject, and
wherein the physiological condition is at least one of:
a heart disorder;
a blood disorder;
a sleep disorder;
a blood pressure disorder;
an organ disorder;
a metabolic disorder;
a neoplastic disorder;
a neurologic disorder;
a psychological or psychiatric disorder;
a traumatic injury;
a hormonal disorder;
a pulmonary disorder;
an infectious disease;
an immunologic disorder;
a digestive disorder;
a reaction to medication; or
a toxin or toxicant exposure

11. The system of claim 10, wherein the physiological condition is a heart disorder, and the heart disorder is at least one of:
an arrhythmia;
atrial fibrillation;
ventricular fibrillation; or
tachycardia.

12. The system of claim 1, wherein the first class is indicative of a medical condition of a subject, and the second class is indicative of not the medical condition of the subject, and
wherein the medical condition is at least one of:
a psychological condition; or
a physiological condition.

13. The system of claim 1, wherein the computing device is further configured to:
fuse together the first data model with the second data model based on the recommendation to create a fused data model;
receive an indication that an event has occurred;
based on the indication that the event has occurred, utilize at least one of the fused data model, the first data model, or the second data model.

14. The system of claim 1, wherein the indication is a user input.

15. The system of claim 13, wherein the event is at least one of:
a low battery signal; or
an emergency indication.

16. A patient evaluation system being used across a hospital to evaluate, monitor, or determine a medical condition of multiple patients, the system comprising:
a processor device;
a display in communication with the processor device;
wherein the processor device is configured to:
receive a plurality of data models, each data model being representative of a first class and a second class, each of the data models being configured to predict a first characteristic that is indicative of either of the first class or the second class;
select a plurality of pairs of data models, each pair of data models being of the plurality of data models;
determine or retrieve, for each pair of data models, a first accuracy of one of the data models within the pair of data models and a second accuracy of the other data model within the data models;
determine or retrieve, for each pair of data models, a first correlation between the pair of data models for the first class, and a second correlation between the pair of data models for the second class;
utilize, for each pair of data models, the first accuracy, the second accuracy, the first correlation, and the second correlation to determine a recommendation for fusing the pair of data models;
and based on the recommendation for or against fusing the pair of data models at least one of:
adjust an operation of the patient monitoring system, or a system in communication with the patient monitoring system, wherein adjust an operation includes at least one of:
the processor device transmitting a notification to the system; or
the processor device fusing one or more pairs of data models;
the processor device notifying or activating the system being a paging system of a doctor;
generate a report that includes, for each pair of data models, the corresponding recommendation for or against fusing the pair of data models, and present, to the display, the report that includes the recommendation for or against fusing each pair of data models;
store, for each pair of data models, the recommendation for or against fusing the pair of data models, in a computer readable memory.

17. The system of claim 16, wherein the plurality of pairs of data models is a number of pairs of data models, the number of pairs data models being greater than 1000.

18. The system of claim 17, wherein the number of pairs of data models being greater than 100,000.

19. The system of claim 18, wherein the number of pairs of data models being greater than 1,000,000.

20. The system of claim 16, wherein the first class is indicative of a physiological condition, and the second class is indicative of not the physiological condition, and
wherein each data model within each pair of data models includes a variable, and
wherein one data model within each pair of data models is only a variable.

21-63. (canceled)
